(19) United States
(12) Patent Application Publication
(10) Pub. No.: US 2011/0150980 A1
(43) Pub. Date: Jun. 23, 2011

(54) CGRP ANALOG

(75) Inventors: George Lee Southard, Sanibel, FL (US); Jeffrey Lee Southard, Olathe, KS (US)

(73) Assignee: VasoGenix Pharmaceuticals, Inc., Lenexa, KS (US)

(21) Appl. No.: 12/949,565

(22) Filed: Nov. 18, 2010

Related U.S. Application Data
(60) Provisional application No. 61/262,299, filed on Nov. 18, 2009.

(30) Foreign Application Priority Data
Nov. 26, 2009 (US) PCT/US2009/003097

(51) Int. Cl. A61K 38/17; A61K 9/14; A61K 9/127; A61P 9/04; A61P 25/06; A61P 13/12; A61P 9/12; A61P 9/00; A61P 9/10; A61P 15/00

(52) U.S. Cl. 424/450; 424/484; 424/486; 514/21.3; 514/16.4; 514/17.7; 514/15.4; 514/15.7

(57) ABSTRACT

Sequence modified calcitonin gene related peptide (CGRP) compounds are useful for treating heart failure, stroke, hemorrhagic shock, or migraine, improving renal function, preventing or delaying the advancement of heart failure into advanced stages, treating angina, controlling pulmonary hypertension, counteracting ischemia due to a myocardial infarction, preventing vasospasms during angioplasty, preventing reocclusion of blood vessels during and/or after angioplasty, stent insertion, or the implantation of a vascular grafts, and for treating male impotence and female sexual arousal disorder.

### CLAIM OF PRIORITY

This application claims the benefit of priority under 35 U.S.C. §119(e) to U.S. Provisional Application No. 61/262,299, filed Nov. 18, 2009. This application also claims the benefit of priority to PCT/US2009/003097, filed May 19, 2009, and published as WO 2009/142727 on Nov. 26, 2009, which claims the benefit of priority under 35 U.S.C. §119(e) to U.S. Provisional Application No. 61/054,337, filed May 19, 2008, the benefit of priority of each of which is claimed hereby, and each of which is incorporated by reference herein in its entirety.

### BACKGROUND

### Field of Invention

The present invention provides sequence modified calcitonin gene related peptides (CGRP), pharmaceutical preparations containing these peptides, and methods for their use in treating a variety of medical disorders.

Calcitonin Gene Related Peptide (“CGRP”) is a naturally occurring peptide in several mammalian species including humans where it occurs in two forms. Human alpha CGRP(H-α-CGRP) and beta CGRP(H-β-CGRP) are each 37 amino acid containing peptides having vasodilation and cardioprotectant properties. The two peptides differ only by three amino acids. The amino acid sequence differences between the alpha and beta forms can be found at the 3, 22, and 25 positions. The alpha form differs from the beta form by substitution of aspartic acid (D) for asparagine (N) at position 3; valine (V) for methionine (M) at position 22; and asparagine (N) for serine (S) at position 25. The alpha and beta forms of CGRP have similar biological functions. In the osteoclast bone resorption assay, both peptides were equipotent.

H-α-CGRP has the primary sequence ACDTATCVTHRLAGLLSRSGGVVKNNFVPTNVGSKAF-NH₂ (SEQ ID NO:1), the carboxy-terminal phenylalanine being in the form of a primary carboxamide.

H-β-CGRP has the primary sequence ACNTATCVTHRLAGLLSRSGGMVKSNFVPTNVGSKAF-NH₂ (SEQ ID NO:2). See M. Zaidi, et al. (1990), “Structure activity relationship of human calcitonin-gene-related-peptide,” Biochem. J., 269(3), 775-780.

Both forms of CGRP are present in plasma, cerebrospinal fluid, and the spinal cord (Wimalawansa, S. J., Morris, H. R., MacIntyre, I. J., Mol. Endocrinol., 3:247 (1989)). Both forms have been isolated and fully characterized by amino acid sequencing and fast atom bombardment-mass spectrometry (FABMS) (Wimalawansa, S. J., Morris, H. R., Etienne, A., Blench, I., Panico, M., and MacIntyre, I., “Isolation, purification and characterization of β-hCGRP from human spinal cord”, Biochem. Biophys. Res. Commun., 167:993 (1990); Steenberg, et al. FEBS Letts., 183:403 (1985); incorporated herein by reference. Genes for CGRP have been identified on chromosome 11 (Hoopener, et al., Hum. Gen., 70:259 (1985)). CGRP receptors have been isolated and purified, and monoclonal antibodies have been raised against these purified receptors (Wimalawansa, S. J., “Isolation and characterization of calcitonin gene-related peptide receptors and raising monoclonal antibodies”, Annals of New York Academy of Sciences, 657:70-87 (1992); Wimalawansa, S. J., Gunasekera, R. D., Zhang, F., “Isolation, purification, and characterization of calcitonin gene-related peptide receptor”, Peptides, 14:691-699 (1993); and Proceedings, First International Symposium on Calcitonin Gene-Related Peptide”, Graz., Austria, Regul., Peptides, 14:691 (1993). All of the above documents are incorporated herein by reference in their entirety.

CGRP is the most potent naturally occurring vasodilator peptide in the human body. It is distributed throughout the central and peripheral nervous systems, and is found in areas that are known to be involved in cardiovascular function (Wimalawansa, S., Critical Reviews in Neurobiology, 11:167-239 (1997)). Peripherally, CGRP is found in the heart, particularly in association with the sinoatrial and atrioventricular nodes. In addition, CGRP is found in nerve fibers that form a dense periadventitial network throughout the peripheral vascular system, including the cerebral, coronary, and renal arteries. CGRP has prominent cardiovascular effects, including vasodilation and positive chronotropic and inotropic effects, which may play an important role in normal cardiovascular function (Wimalawansa, S., Endocrine Reviews, 17:208:217 (1996)).

### SUMMARY OF THE INVENTION

The present invention is directed to compounds analogous to human isoforms of CGRP, to conjugates, topical compositions, microparticulate or liposomal delivery systems, pharmaceutical compositions, and sustained release delivery systems, comprising the peptide or peptide-like analogs of CGRP of the invention, to methods of treatment of various malconditions comprising administration of an effective amount of a compound of the invention to a mammal in need thereof, and to kits adapted for administration of compounds of the invention.

In various embodiments, the invention provides a sequence modified calcitonin gene related peptide (CGRP) or pharmaceutically acceptable salt thereof, of any of structures (I) to (XII)

(I; SEQ ID NO: 3)
A C N T A T C V T H R L A G L L S R S G G V V K N
N F V P T N V G S K A F-NH₂
(II; SEQ ID NO: 4)
A C D T A T C V T H R L A G L L S R S G G M V K N
N F V P T N V G S K A F-NH₂
(III; SEQ ID NO: 5)
A C D T A T C V T H R L A G L L S R S G G V V K S
N F V P T N V G S K A F-NH₂
(IV; SEQ ID NO: 6)
A C N T A T C V T H R L A G L L S R S G G M V K N
N F V P T N V G S K A F-NH₂
(V; SEQ ID NO: 7)
A C N T A T C V T H R L A G L L S R S G G V V K S
N F V P T N V G S K A F-NH₂
(VI; SEQ ID NO: 8)
A C D T A T C V T H R L A G L L S R S G G M V K S
N F V P T N V G S K A F-NH₂
(VII; SEQ ID NO: 9)
A C N T A T C V T H R L A D L L S R S G G V V K N
N F V P T N V G S K A F-NH₂
(VIII; SEQ ID NO: 10)
A C D T A T C V T H R L A D L L S R S G G M V K N
N F V P I N V G S K A F-NH₂
(IX; SEQ ID NO: 11)
A C D T A T C V T H R L A D L L S R S G G V V K S
N F V P T N V G S K A F-NH₂
(X; SEQ ID NO: 12)
A C N T A T C V T H R L A D L L S R S G G M V K N
N F V P T N V G S K A F-NH₂
(XI; SEQ ID NO: 13)
A C N T A T C V T H R L A D L L S R S G G V V K S
N F V P T N V G S K A F-NH₂
(XII; SEQ ID NO: 14)
A C D T A T C V T H R L A D L L S R S G G M V K S
N F V P T N V G S K A F-NH₂

In preferred embodiments, the invention provides a CGRP analog that is Peptide V or pharmaceutically acceptable salt thereof. In various embodiments, the invention provides an N-terminal acylated form of any of formulas (I)-(XII) of the invention, wherein the acyl group comprises a formyl group, a (C₁-C₂₀)C(═O)— group or an aroyl group. For example, the acyl group can be formyl, acetyl, a (C₁₂-C₁₈)-fatty acyl, or benzoyl, providing the corresponding N-terminal amide.

In various embodiments, the invention provides an inverso peptide with respect to formulas (I)-(XII) of any of formulas (IA)-(XIIA):

(IA)
(A C N T A T C V T H R L A G L L S R S G G V V K N
N F V P T N V G S K A F-NH₂)-all-D
(IIA)
(A C D T A T C V T H R L A G L L S R S G G M V K N
N F V P T N V G S K A F-NH₂)-all-D
(IIIA)
(A C D T A T C V T H R L A G L L S R S G G V V K S
N F V P T N V G S K A F-NH₂)-all-D
(IVA)
(A C N T A T C V T H R L A G L L S R S G G M V K N
N F V P T N V G S K A F-NH₂)-all-D
(VA)
(A C N T A T C V T H R L A G L L S R S G G V V K S
N F V P T N V G S K A F-NH₂)-all-D
(VIA)
(A C D T A T C V T H R L A G L L S R S G G M V K S
N F V P T N V G S K A F-NH₂)-all-D
(VIIA)
(A C N T A T C V T H R L A D L L S R S G G V V K N
N F V P T N V G S K A F-NH₂)-all-D
(VIIIA)
(A C D T A T C V T H R L A D L L S R S G G M V K N
N F V P T N V G S K A F-NH₂)-all-D
(IXA)
(A C D T A T C V T H R L A D L L S R S G G V V K S
N F V P T N V G S K A F-NH₂)-all-D
(XA)
(A C N T A T C V T H R L A D L L S R S G G M V K N
N F V P T N V G S K A F-NH₂)-all-D
(XIA)
(A C N T A T C V T H R L A D L L S R S G G V V K S
N F V P T N V G S K A F-NH₂)-all-D
(XIIA)
(A C D T A T C V T H R L A D L L S R S G G M V K S
N F V P T N V G S K A F-NH₂)-all-D.

In various embodiments, the invention provides an N-terminal acylated form of any of formulas (IA)-(XIIA) of the invention, wherein the acyl group comprises a formyl group, a (C₁-C₂₀)C(═O)— group or an aroyl group. For example, the acyl group can be formyl, acetyl, a (C₁₂-C₁₈)-fatty acyl, or benzoyl, providing the corresponding N-terminal amide.

In various embodiments, the invention provides a retro or retro-inverso peptide with respect to formulas (I)-(XII) of any of formulas (IB)-(XIIB) or (IC)-(XIIC) respectively:

(IB; SEQ ID NO: 15)
F A K S G V N T P V F N N K V V G G S R S L L G A
L R H T V C T A T N C A-NH₂
(IC)
(F A K S G V N T P V F N N K V V G G S R S L L G A
L R H T V C T A T N C A-NH₂)all-D
(IIB; SEQ ID NO: 16)
F A K S G V N T P V F N N K V M G G S R S L L G A
L R H T V C T A T D C A-NH₂
(IIC)
(F A K S G V N T P V F N N K V M G G S R S L L G A
L R H T V C T A T D C A-NH₂)-all-D
(IIIB; SEQ ID NO: 17)
F A K S G V N T P V F N S K V V G G S R S L L G A
L R H T V C T A T D C A-NH₂
(IIIC)
(F A K S G V N T P V F N S K V V G G S R S L L G A
L R H T V C T A T D C A-NH₂)-all-D
(IVB; SEQ ID NO: 18)
F A K S G V N T P V F N N K V M G G S R S L L G A
L R H T V C T A T N C A-NH₂
(IVC)
(F A K S G V N T P V F N N K V M G G S R S L L G A
L R H T V C T A T N C A-NH₂)-all-D
(VB; SEQ ID NO: 19)
F A K S G V N T P V F N S K V V G G S R S L L G A
L R H T V C T A T N C A-NH₂
(VC)
(F A K S G V N T P V F N S K V V G G S R S L L G A
L R H T V C T A T N C A-NH₂)-all-D
(VIB; SEQ ID NO: 20)
F A K S G V N T P V F N S K V M G G S R S L L G A
L R H T V C T A T D C A-NH₂
(VIC)
(F A K S G V N T P V F N S K V M G G S R S L L G A
L R H T V C T A T D C A-NH₂)-all-D
(VIIB; SEQ ID NO: 21)
F A K S G V N T P V F N N K V V G G S R S L L D A
L R H T V C T A T N C A-NH₂
(VIIC)
(F A K S G V N T P V F N N K V V G G S R S L L D A
L R H T V C T A T N C A-NH₂)-all-D
(VIIIB; SEQ ID NO: 22)
F A K S G V N T P V F N N K V M G G S R S L L D A
L R H T V C T A T D C A-NH₂
(VIIIC)
(F A K S G V N T P V F N N K V M G G S R S L L D A
L R H T V C T A T D C A-NH₂)-all-D
(IXB; SEQ ID NO: 23)
F A K S G V N T P V F N S K V V G G S R S L L D A
L R H T V C T A T D C A-NH₂
(IXC)
(F A K S G V N T P V F N S K V V G G S R S L L D A
L R H T V C T A T D C A-NH₂)-all-D
(XB; SEQ ID NO: 24)
F A K S G V N T P V F N N K V M G G S R S L L D A
L R H T V C T A T N C A-NH₂
(XC)
(F A K S G V N T P V F N N K V M G G S R S L L D A
L R H T V C T A T N C A-NH₂)-all-D
(XIB; SEQ ID NO: 25)
F A K S G V N T P V F N S K V V G G S R S L L D A
L R H T V C T A T N C A-NH₂
(XIC)
(F A K S G V N T P V F N S K V V G G S R S L L D A
L R H T V C T A T N C A-NH₂)-all-D
(XIIB; SEQ ID NO: 26)
F A K S G V N T P V F N S K V M G G S R S L L D A
L R H T V C T A T D C A-NH₂
(XIIC)
(F A K S G V N T P V F N S K V M G G S R S L L D A
L R H T V C T A T D C A-NH₂)-all-D,

or a retro or a retro-inverso form of α-CGRP comprising a sequence-modified CGRP of the respective formulas:

(SEQ ID NO: 27)
F A K S G V N T P V F N N K V V G G S R S L L G
A L R H T V C T A T D C A-NH₂
(F A K S G V N T P V F N N K V V G G S R S L L G
A L R H T V C T A T D C A-NH₂) all-D;
or
an AA₁₄ G to D mutation sequence thereof of the
respective formulas
(SEQ ID NO: 60)
F A K S G V N T P V F N N K V V G G S R S L L D A
L R H T V C T A T D C A-NH₂
(F A K S G V N T P V F N N K V V G G S R S L L D
A L R H T V C T A T D C A-NH₂) all-D;
or
a retro or a retro-inverso form of β-CGRP of the
respective formulas:
(SEQ ID NO: 29)
F A K S G V N T P V F N S K V M G G S R S L L G A
L R H T V C T A T N C A-NH₂
(F A K S G V N T P V F N S K V M G G S R S L L G
A L R H T V C T A T N C A-NH₂) all-D
or
an AA₁₄ G to D mutation sequence thereof of the
respective formulas
(SEQ ID NO: 28)
F A K S G V N T P V F N S K V M G G S R S L L D A
L R H T V C T A T N C A-NH₂
(F A K S G V N T P V F N S K V M G G S R S L L D
A L R H T V C T A T N C A-NH₂) all-D,

or a pharmaceutically acceptable salt thereof.

In various embodiments, the invention provides an N-terminal acylated form of any of formulas (IB)-(XIIB) or (IC)-(XIIC), or of the retro or retro-inverso forms of α-CGRP or of β-CGRP, or of the AA₁₄ (original numbering) G to D mutation sequences corresponding thererto, of the invention, wherein the acyl group comprises a formyl group, a (C₁-C₂₀)C(═O)— group or an aroyl group, providing the corresponding N-terminal amide. For example, the acyl group can be formyl, acetyl, a (C₁₂-C₁₈)-fatty acyl, or benzoyl.

In various embodiments, the invention provides an AA1-des-alanyl sequence modified calcitonin gene related peptide (CGRP), of any of structures (XIII) to (XXIV)

(XIII; SEQ ID NO: 30)
C N T A T C V T H R L A G L L S R S G G V V K N N
F V P T N V G S K A F-NH₂
(XIV; SEQ ID NO: 31)
C D T A T C V T H R L A G L L S R S G G M V K N N
F V P T N V G S K A F-NH₂
(XV; SEQ ID NO: 32)
C D T A T C V T H R L A G L L S R S G G V V K S N
F V P T N V G S K A F-NH₂
(XVI; SEQ ID NO: 33)
C N T A T C V T H R L A G L L S R S G G M V K N N
F V P T N V G S K A F-NH₂
(XVII; SEQ ID NO: 34)
C N T A T C V T H R L A G L L S R S G G V V K S N
F V P T N V G S K A F-NH₂
(XVIII; SEQ ID NO: 35)
C D T A T C V T H R L A G L L S R S G G M V K S N
F V P T N V G S K A F-NH₂
(XIX; SEQ ID NO: 36)
C N T A T C V T H R L A D L L S R S G G V V K N N
F V P T N V G S K A F-NH₂
(XX; SEQ ID NO: 37)
C D T A T C V T H R L A D L L S R S G G M V K N N
F V P T N V G S K A F-NH₂
(XXI; SEQ ID NO: 38)
C D T A T C V T H R L A D L L S R S G G V V K S N
F V P T N V G S K A F-NH₂
(XXII; SEQ ID NO: 39)
C N T A T C V T H R L A D L L S R S G G M V K N N
F V P T N V G S K A F-NH₂
(XXIII; SEQ ID NO: 40)
C N T A T C V T H R L A D L L S R S G G V V K S N
F V P T N V G S K A F-NH₂
(XXIV; SEQ ID NO: 41)
C D T A T C V T H R L A D L L S R S G G M V K S N
F V P T N V G S K A F-NH₂;

or a retro, inverso, or retro-inverso form thereof, or a pharmaceutically acceptable salt thereof.

In various embodiments, the invention provides an N-terminal acylated form of any of formulas (XIII)-(XXIV) of the invention, wherein the acyl group comprises a formyl group, a (C₁-C₂₀)C(═O)— group or an aroyl group. For example, the acyl group can be formyl, acetyl, a (C₁₂-C₁₈)-fatty acyl, or benzoyl.

In various embodiments, the invention provides an AA1-AA7 deleted sequence modified calcitonin gene related peptide (CGRP) or pharmaceutically acceptable salt thereof, of any of structures (XXV) to (XXXVI)

(XXV; SEQ ID NO: 42)
V T H R L A G L L S R S G G V V K N N F V P T N V
G S K A F-NH₂
(XXVI; SEQ ID NO: 52)
V T H R L A G L L S R S G G M V K N N F V P T N V
G S K A F-NH₂
(XXVII; SEQ ID NO: 53)
V T H R L A G L L S R S G G V V K S N F V P T N V
G S K A F-NH₂
(XXX; SEQ ID NO: 43)
V T H R L A G L L S R S G G M V K S N F V P T N V
G S K A F-NH₂
(XXXI; SEQ ID NO: 44)
V T H R L A D L L S R S G G V V K N N F V P T N V
G S K A F-NH₂
(XXXII; SEQ ID NO: 56)
V T H R L A D L L S R S G G M V K N N F V P T N V
G S K A F-NH₂
(XXXIII; SEQ ID NO: 57)
V T H R L A D L L S R S G G V V K S N F V P T N V
G S K A F-NH₂
(XXXVI; SEQ ID NO: 45)
V T H R L A D L L S R S G G M V K S N F V P T N V
G S K A F-NH₂.

In various embodiments, the invention provides an N-terminal acylated form of any of formulas (XXV)-(XXXVI) of claim 13, wherein the acyl group comprises a formyl group, a (C₁-C₂₀)C(═O)— group or an aroyl group. For example, the acyl group can be formyl, acetyl, a (C₁₂-C₁₈)-fatty acyl, or benzoyl.

In various embodiments, the invention provides a retro or retro-inverso peptide with respect to formulas (XXV)-(XXXVI) of any of formulas (XXVB)-(XXXVIB) or (XXVC)-(XXXVIC) respectively:

(XXVB; SEQ ID NO: 46)
F A K S G V N T P V F N N K V V G G S R S L L G A
L R H T V-NH₂
(XXVC)
(F A K S G V N T P V F N N K V V G G S R S L L G A
L R H T V-NH₂)all-D
(XXVIB; SEQ ID NO: 54)
F A K S G V N T P V F N N K V M G G S R S L L G A
L R H T V-NH₂
(XXVIC)
(F A K S G V N T P V F N N K V M G G S R S L L G A
L R H T V-NH₂)-all-D
(XXVIIB; SEQ ID NO: 55)
F A K S G V N T P V F N S K V V G G S R S L L G A
L R H T V-NH₂
(XXVIIC)
(F A K S G V N T P V F N S K V V G G S R S L L G A
L R H T V-NH₂)-all-D
(XXXB; SEQ ID NO: 47)
F A K S G V N T P V F N S K V M G G S R S L L G A
L R H T V-NH₂
(XXXC)
(F A K S G V N T P V F N S K V M G G S R S L L G A
L R H T V-NH₂)-all-D
(XXXIB; SEQ ID NO: 48)
F A K S G V N T P V F N N K V V G G S R S L L D A
L R H T V-NH₂
(XXXIC)
(F A K S G V N T P V F N N K V V G G S R S L L D A
L R H T V-NH₂)-all-D
(XXXIIB; SEQ ID NO: 58)
F A K S G V N T P V F N N K V M G G S R S L L D A
L R H T V-NH₂
(XXXIIC)
(F A K S G V N T P V F N N K V M G G S R S L L D A
L R H T V-NH₂)-all-D
(XXXIIIB; SEQ ID NO: 59)
F A K S G V N T P V F N S K V V G G S R S L L D A
L R H T V-NH₂
(XXXIIIC)
(F A K S G V N T P V F N S K V V G G S R S L L D A
L R H T V-NH₂)-all-D
(XXXVIB; SEQ ID NO: 49)
F A K S G V N T P V F N S K V M G G S R S L L D A
L R H T V-NH₂
(XXXVIC)
(F A K S G V N T P V F N S K V M G G S R S L L D A
L R H T V-NH₂)-all-D

or a pharmaceutically acceptable salt thereof.

In various embodiments, the invention provides an N-terminal acyl form of any of formulas (XXVB)-(XXXVIB) or (XXVC)-(XXXXVIC) of the invention, wherein the acyl group comprises a formyl group, a (C₁-C₂₀)C(═O)— group or an aroyl group. For example, the acyl group can be formyl, acetyl, a (C₁₂-C₁₈)-fatty acyl, or benzoyl.

In various embodiments, the invention provides a terminally modified retro-inverso peptide with respect to formulas (I)-(XII) of the formula (XXXVII)-(XLVIII)

(XXXVII)
X(O═)C—CH(CH₂C₆H₅)C(O)-(A K S G V N T P V F N N K
V V G G S R S L L G A L R H T V C T A T N C)-NHCH
(CH₃)NH₂) all-D
(XXXVIII)
X(O═)C—CH(CH₂C₆H₅)C(O)-A K S G V N T P V F N N K
V M G G S R S L L G A L R H T V C T A T D C)-NHCH
(CH₃)NH₂)all-D
(XXXIX)
X(O═)C—CH(CH₂C₆H₅)C(O)-A K S G V N T P V F N S K
V V G G S R S L L G A L R H T V C T A T D C)-NHCH
(CH₃)NH₂)all-D
(XL)
X(O═)C—CH(CH₂C₆H₅)C(O)-A K S G V N T P V F N N K
V M G G S R S L L G A L R H T V C T A T N C)-NHCH
(CH₃)NH₂)all-D
(XLI)
X(O═)C—CH(CH₂C₆H₅)C(O)-A K S G V N T P V F N S K
V V G G S R S L L G A L R H T V C T A T N C)-NHCH
(CH₃)NH₂)all-D
(XLII)
X(O═)C—CH(CH₂C₆H₅)C(O)-A K S G V N T P V F N S K
V M G G S R S L L G A L R H T V C T A T D C)-NHCH
(CH₃)NH₂)all-D
(XLIII)
X(O═)C—CH(CH₂C₆H₅)C(O)-A K S G V N T P V F N N K
V V G G S R S L L D A L R H T V C T A T N C)-NHCH
(CH₃)NH₂)all-D
(XLIV)
X(O═)C—CH(CH₂C₆H₅)C(O)-A K S G V N T P V F N N K
V M G G S R S L L D A L R H T V C T A T D C)-NHCH
(CH₃)NH₂)all-D
(XLV)
X(O═)C—CH(CH₂C₆H₅)C(O)-A K S G V N T P V F N S K
V V G G S R S L L D A L R H T V C T A T D C)-NHCH
(CH₃)NH₂)all-D
(XLVI)
X(O-)C—CH(CH₂C₆H₅)C(O)-A K S G V N T P V F N N K
V M G G S R S L L D A L R H T V C T A T N C)-NHCH
(CH₃)NH₂)all-D
(XLVII)
X(O═)C—CH(CH₂C₆H₅)C(O)-A K S G V N T P V F N S K
V V G G S R S L L D A L R H T V C T A T N C)-NHCH
(CH₃)NH₂)all-D
(XLVIII)
X(O═)C—CH(CH₂C₆H₅)C(O)-A K S G V N T P V F N S K
V M G G S R S L L D A L R H T V C T A T D C)-NHCH
(CH₃)NH₂)all-D,

or terminally modified retro-inverso peptide with respect to α-CGRP of the formula

(XLVII)
X(O═)C-CH(CH₂C₆H₅)C(O)-(A K S G V N T P V F N N
K V V G G S R S L L G A L R H T V C T A T D C)-
NHCH(CH₃)NH₂) all-D,

or a sequence modified form thereof of the formula

X(O═)C-CH(CH₂C₆H₅)C(O)-(A K S G V N T P V F N N
K V V G G S R S L L D A L R H T V C T A T D C)-
NHCH(CH₃)NH₂) all-D

or terminally modified retro-inverso peptide with respect to β-CGRP of the formula

X(O═)C-CH(CH₂C₆H₅)C(O)-(A K S G V N T P V F N S
K V M G G S R S L L G A L T H T V C T A T N C)-
NHCH(CH₃)NH₂) all-D

or a sequence modified form thereof of the formula

X(O═)C-CH(CH₂C₆H₅)C(O)-(A K S G V N T P V F N S
K V M G G S R S L L D A L T H T V C T A T N C)-
NHCH(CH₃)NH₂) all-D

wherein X is NH₂ or OH, or a pharmaceutically acceptable salt thereof.

In various embodiments, the invention provides a peptide of any one of formulas (XXXVII)-(XLVIII) wherein the NHCH(CH₃)NH₂ moiety is further modified by acylation to provide a corresponding peptide wherein the moiety is of the formula NHCH(CH₃)NH—C(═O)Y, wherein Y is H, (C₁-C₂₀)alkyl, or aryl.

In various embodiments, the invention provides a terminally modified retro-inverso peptide with respect to formulas (XXV)-(XXXVI) of the formula

(XLIX)-(LX)
(XLIX)
X(O═)C-CH(CH₂C₆H₅)C(O)-(A K S G V N T P V F N N K
V V G G S R S L L G A L R H T V)-NHCH(CH₃)NH₂)
all-D
(L)
X(O═)C-CH(CH₂C₆H₅)C(O)-(A K S G V N T P V F N N K
V M G G S R S L L G A L R H T V)-NHCH(CH₃₎NH₂)
all-D
(LI)
X(O═)C-CH(CH₂C₆H₅)C(O)-(A K S G V N T P V F N S K
V V G G S R S L L G A L R H T V)-NHCH(CH₃)NH₂)
all-D
(LIV)
X(O═)C-CH(CH₂C₆H₅)C(O)-(A K S G V N T P V F N S K
V M G G S R S L L G A L R H T V)-NHCH(CH₃)NH₂)
all-D
(LV)
X(O═)C-CH(CH₂C₆H₅)C(O)-(A K S G V N T P V F N N K
V V G G S R S L L D A L R H T V)-NHCH(CH₃)NH₂)
all-D
(LVI)
X(O═)C-CH(CH₂C₆H₅)C(O)-(A K S G V N T P V F N N K
V M G G S R S L L D A L R H T V)-NHCH(CH₃)NH₂)
all-D
(LVII)
X(O═)C-CH(CH₂C₆H₅)C(O)-(A K S G V N T P V F N S K
V V G G S R S L L D A L R H T V)-NHCH(CH₃)NH₂)
all-D
(LX)
X(O═)C-CH(CH₂C6H₅)C(O)-(A K S G V N T P V F N S K
V M G G S R S L L D A L R H T V)-NHCH(CH₃)NH₂)
all-D,

wherein X is NH₂ or OH, or a pharmaceutically acceptable salt thereof.

In various embodiments, the invention provides a peptide of any one of formulas (XLIX)-(LX) wherein the NHCH(CH₃)NH₂ moiety is further modified by acylation to provide a corresponding peptide wherein the moiety is of the formula NHCH(CH₃)NH—C(═O)Y, wherein Y is H, (C₁-C₂₀)alkyl, or aryl.

In various embodiments, a sequence modified calcitonin gene related peptide (CGRP) of the invention or pharmaceutically acceptable salt thereof can also be described as follows: ACX₁TATCVTHRLAX₂LLSRSGGX₃VKX₄NFVPTNVGSKAF-NH₂ (SEQ ID NO:50), wherein X₁ is aspartic acid or asparagine, X₂ is glycine, aspartic acid or glutamic acid, X₃ is valine or methionine and X₄ is asparagine or serine. With respect to the native CGRP structures, in various embodiments the sequence modified CGRP peptides of the invention include one or more amino acid residue substitutions at positions 3, 14, 22, or 25 from the amino terminal alanine residue.

In some embodiments, peptides are provided wherein the alanine at position 1 in the sequence modified calcitonin gene related peptide is deleted from the sequence such that the alanine is replaced by a hydrogen on the alpha amine group of the cysteine at position 2, as shown above.

In some embodiments, deletion sequences consisting of residues 8-37, with mutations at positions 14, 22 or 25 with respect to the original sequence (i.e., positions 7, 15, or 18 with respect to the truncated sequence) are provided, as shown above.

In various embodiments, retro, inverso, and retro-inverso forms of all these sequence modified normal CGRP peptides are provided. In various embodiments, modified forms of retro-inverso sequence modified CGRPs are provided, as shown above.

The sequence modified calcitonin gene-related peptides may be purified, isolated, purified and isolated, or used in any form. In some embodiments, the sequence modified CGRP is bis-acetylated or N-acetylated, as shown above, (as in Zaidi et al., Biochem J. 1990 Aug. 1; 269(3):775-80, the disclosure of which is incorporated herein by reference in its entirety).

The present invention also provides a pharmaceutical composition comprising a pharmaceutical excipient, diluent, or carrier in association with one or more sequence modified calcitonin gene-related peptides (CGRP) or a pharmaceutically acceptable salt thereof selected from the group consisting of structures (I) to (XII), (IA) to (XIIA), (IB) to (XIIB), (IC) to (XIIC), (XIII) to (XXIV), (XXV) to (XXXVI), (XXXVII) to (XLVIII), and (XLIX) to (LX), as shown above. In a preferred embodiment, a pharmaceutical composition is provided comprising a pharmaceutical excipient, diluent, or carrier in association with Peptide V.

In various embodiments, a pharmaceutical composition comprising a pharmaceutical excipient, diluent, or carrier in association with one or more sequence modified calcitonin gene-related peptides (CGRP) can comprise a peptide as shown or a pharmaceutically acceptable salt thereof: ACX₁TATCVTHRLAX₂LLSRSGGX₃VKX₄NFVPTNVGSKAF-NH₂ (SEQ ID NO:50) wherein X₁ is aspartic acid or asparagine, X₂ is glycine, aspartic acid or glutamic acid, X₃ is valine or methionine and X₄ is asparagine or serine.

The present invention further provides a method of preventing, treating, reducing the risks, occurrence, progression, symptoms, or effects of: heart failure, ischemia, myocardial infarction, renal failure, stroke, migraine, pulmonary hypertension, hemorrhagic shock, angina, vasospasms, male impotence, or female sexual arousal disorder comprising:

administering to a subject in need thereof, an effective amount of one or more of an sequence modified calcitonin gene related peptide (CGRP) sequence modified calcitonin gene related peptide (CGRP) or a pharmaceutically acceptable salt thereof selected from the group consisting of structures (I) to (XII)), (IA) to (XIIA), (IB) to (XIIB), (IC) to (XIIC), (XIII) to (XXIV), (XXV) to (XXXVI), (XXXVII) to (XLVIII), and (XLIX) to (LX), as shown above. In preferred embodiment, the method comprises administering an effective amount of peptide structure V.

An effective amount of the following sequence modified calcitonin gene related peptide (CGRP) or a pharmaceutically acceptable salt thereof can also be administered to a subject in need thereof: ACX₁TATCVTHRLAX₂LLSRSGGX₃VKX₄NFVPTNVGSKAF-NH₂

(SEQ ID NO:50) wherein X₁ is aspartic acid or asparagine, X₂ is glycine, aspartic acid or glutamic acid, X₃ is valine or methionine and X₄ is asparagine or serine.

The present invention additionally provides a kit comprising a first container comprising a controlled release formulation of sequence modified CGRP, the formulation comprising an amount of sequence modified CGRP or a pharmaceutically acceptable salt thereof effective to treat, reduce the risks, occurrence, or effects of; heart failure, ischemia, myocardial infarction, renal failure, stroke, migraine, pulmonary hypertension, hemorrhagic shock, angina, vasospasms, male impotence, or female sexual arousal disorder,

wherein the sequence modified calcitonin gene related peptide (CGRP) or pharmaceutically acceptable salt thereof is selected from the group consisting of structures (I) to (XII)), (IA) to (XIIA), (IB) to (XIIB), (IC) to (XIIC), (XIII) to (XXIV), (XXV) to (XXXVI), (XXXVII) to (XLVIII), and (XLIX) to (LX), as shown above. In a preferred embodiment, the kit comprises a sequence modified CGRP of peptide structure V.

The kit can also comprise the following sequence modified calcitonin gene related peptide (CGRP) or a pharmaceutically acceptable salt thereof: ACX₁TATCVTHRLAX₂LLSRSGGX₃VKX₄NFVPTNVGSKAF-NH₂ (SEQ ID NO:50) wherein X₁ is aspartic acid or asparagine, X₂ is glycine, aspartic acid or glutamic acid, X₃ is valine or methionine and X₄ is asparagine or serine.

The present invention also provides the above sequence modified CGRP, or pharmaceutically acceptable salts thereof, dispersed in a pharmaceutically acceptable liquid or solid carrier, or expressed directly in vivo by gene therapy techniques, can be administered to mammals, including humans, to influence cardiac and renal function, blood pressure, local and systemic blood flow, immunomodulation, tissue salvage, organ failure, organ rejection, and wound healing in acute and chronic treatment time periods.

The present invention provides methods of treating heart failure, stroke, or migraine, improving renal function, preventing or delaying the advancement of heart failure into advanced stages, treating angina, controlling pulmonary hypertension, counteracting ischemia due to a myocardial infarction, preventing vasospasms during angioplasty, preventing reocclusion of blood vessels during and/or after angioplasty, stent insertion, or the implantation of a vascular grafts, and for treating male impotence and female sexual arousal disorder using a sequence modified CGRP of the invention. Work in relation to the present invention has demonstrated that sequence modified CGRP has unexpected improved activity compared to native CGRP (Example 1) in reducing mean arterial pressure (MAP), diastolic Blood pressure (DBP) and systolic blood pressure (SBP). Furthermore, sequence modified CGRP (Peptide V) showed a reduced increase in heart rate compared to native CGRP (Examples 1 and 2).

The present invention provides methods of treatment comprising administering a flowable composition comprising a biodegradable polymer, a biocompatible solvent and a sequence modified CGRP of the invention to a bodily tissue or fluid in the patient, wherein the amounts of the polymer and the solvent are effective to form the polymer matrix comprising sequence modified CGRP in situ when the formulation contacts the bodily fluid tissue or fluid wherein sequence modified CGRP (see Table 4 and 5, pg. 88, 89) comprises between about 5% and 15% by weight and the sequence modified CGRP is released from the polymer matrix at a rate between about 0.0008 and about 0.080 μg/min/kg body weight over a period of 7 to 180 days; wherein the sequence modified calcitonin gene related peptide (CGRP) is selected from the groups consisting of structures (I) to (XII)), (IA) to (XIIA), (IB) to (XIIB), (IC) to (XIIC), (XIII) to (XXIV), (XXV) to (XXXVI), (XXXVII) to (XLVIII), and (XLIX) to (LX), as shown above. In a preferred embodiment, structure V is the sequence modified CGRP. In some embodiments, the sequence modified CGRP is released at a rate of about 0.024 μg/min/kg body weight, and in other embodiments, the sequence modified CGRP is released at a rate of about 0.016 μg/min/kg body weight.

The present invention further provides methods of treatment comprising: (a) administering sequence modified CGRP to the patient by methods such as parenteral, transdermal, intranasal, sublingual, transmucosal, intra-arterial, oral, intracoronary, intravenous, transmucosal, topical rectal, vaginal, or intradermal delivery for a time and at a dose effective to provide symptomatic relief, prevent exacerbation of symptoms, and/or prevent and/or delay progression of the disease state of heart failure in the patient; and (b) delivering sequence modified CGRP to the patient as a controlled release formulation in an amount effective to provide symptomatic relief, prevent exacerbation of symptoms, and/or prevent and/or delay progression of the disease state of heart failure in the patient; wherein the sequence modified calcitonin gene related peptide (CGRP) is selected from the group consisting of structures (I) to (XII)), (IA) to (XIIA), (IB) to (XIIB), (IC) to (XIIC), (XIII) to (XXIV), (XXV) to (XXXVI), (XXXVII) to (XLVIII), and (XLIX) to (LX), as shown above. In preferred embodiments, the sequence modified CGRP is structure V. Controlled release formulations can be delivered by method such as parenteral, transdermal, intranasal, sublingual, transmucosal, intra-arterial, oral, intracoronary, intravenous, transmucosal, topical rectal, vaginal, or intradermal delivery.

The present invention still further provides methods of treatment comprising: delivering to a human at risk of having a myocardial infarction a controlled release formulation of sequence modified CGRP comprising an amount of sequence modified CGRP effective to prevent or reduce the risk or occurrence of myocardial infarction; wherein the sequence modified calcitonin gene related peptide (CGRP) is selected from the group consisting of structures (I) to (XII)), (IA) to (XIIA), (IB) to (XIIB), (IC) to (XIIC), (XIII) to (XXIV), (XXV) to (XXXVI), (XXXVII) to (XLVIII), and (XLIX) to (LX), as shown above. In preferred embodiments, the sequence modified CGRP is structure V.

The present invention yet further provides a kit comprising a first container comprising a controlled release formulation of sequence modified CGRP, the formulation comprising an amount of sequence modified CGRP effective to treat or prevent heart failure and/or renal failure; wherein the sequence modified calcitonin gene related peptide (CGRP) is selected from the group consisting of structures (I) to (XII)), (IA) to (XIIA), (IB) to (XIIB), (IC) to (XIIC), (XIII) to (XXIV), (XXV) to (XXXVI), (XXXVII) to (XLVIII), and (XLIX) to (LX), as shown above. In preferred embodiments, the sequence modified CGRP is structure V.

The present invention additionally provides a method of counteracting ischemia due to myocardial infarction in a patient, comprising delivering to the patient an amount of sequence modified CGRP effective to provide cardioprotection, reduction in infarction size, reduction in reperfusion injury, symptomatic relief, and/or prevent exacerbation of symptoms, wherein the sequence modified CGRP is delivered to the patient as a controlled release composition; wherein the sequence modified calcitonin gene related peptide (CGRP) is selected from the group consisting of structures (I) to (XII)), (IA) to (XIIA), (IB) to (XIIB), (IC) to (XIIC), (XIII) to (XXIV), (XXV) to (XXXVI), (XXXVII) to (XLVIII), and (XLIX) to (LX), as shown above. In preferred embodiments, the sequence modified CGRP is structure V.

The present invention additionally provides a method for counteracting pathological vasospasms or ischemia in target arteries of a mammal where the target arteries comprise a specialized vascular bed selected from the group consisting of coronary, carotid, and renal arteries, the method comprising administering a pharmaceutical preparation of a compound selected from the group consisting of sequence modified calcitonin gene-related peptide (CGRP) that bind to the CGRP receptor to the mammal in an amount effective to counteract the pathological vasospasm or ischemia, where the vasospasms or ischemia are due to a cause selected from the group consisting of angioplasty, vascular graft, stent insertion, and arterial surgery; wherein the sequence modified calcitonin gene related peptide (CGRP) is selected from the group consisting of structures (I) to (XII), (IA) to (XIIA), (IB) to (XIIB), (IC) to (XIIC), (XIII) to (XXIV), (XXV) to (XXXVI), (XXXVII) to (XLVIII), and (XLIX) to (LX), as shown above. In preferred embodiments, the sequence modified CGRP is structure V.

The present invention additionally provides a method for counteracting vasospasms in a specialized vascular bed, ischemia, or renal failure in a mammal comprising intravenously infusing between about 0.0008 and about 0.080 μg/kg/min of a pharmaceutical preparation of sequence modified calcitonin gene-related peptide in an amount effective to counteract the vasospasms, ischemia, or renal failure, where the vasospasms or ischemia are due to a cause selected from the group consisting of angioplasty, vascular graft, stent insertion, and arterial surgery, wherein the sequence modified calcitonin gene related peptide (CGRP) is selected from the group consisting of structures (I) to (XII)), (IA) to (XIIA), (IB) to (XIIB), (IC) to (XIIC), (XIII) to (XXIV), (XXV) to (XXXVI), (XXXVII) to (XLVIII), and (XLIX) to (LX), as shown above. In preferred embodiments, the sequence modified CGRP is structure V. In some embodiments, about 0.52 pmol/kg/min, about 0.75 pmol/kg/min, about 1 pmol/kg/min, about 2 pmol/kg/min, about 3 pmol/kg/min, about 4 pmol/kg/min, about 5 pmol/kg/min, about 6 pmol/kg/min, about 7 pmol/kg/min, about 8 pmol/kg/min, about 9 pmol/kg/min, or about 10 pmol/kg/min of the pharmaceutical preparation of sequence modified CGRP is administered intravenously. In some embodiments, between about 0.0008 and about 0.080 μg/kg/min is administered intravenously. In some embodiments, about 0.0008 μg/kg/min, about 0.008 μg/kg/min, about 0.016 μg/kg/min, about 0.024 μg/kg/min, or about 0.080 μg/kg/min of the pharmaceutical preparation of sequence modified CGRP is administered intravenously.

The present invention additionally provides a method for counteracting pathological vasospasms, organ ischemia due to a degree of narrowing or an obstruction, or renal failure of a mammal comprising administering a pharmaceutical preparation of sequence modified calcitonin gene-related peptide to an artery through a catheter, in a bolus dose of between about 0.25-1 nmol and in an infusion of about 0.52 to about 10 pmole/kg/min, and intravenously in an infusion of about 0.52 to about 10 pmole/kg/min or about 50 to about 500 pmol/kg/hr, wherein the sequence modified calcitonin gene-related peptide is selected from the group consisting of structures (I) to (XII)), (IA) to (XIIA), (IB) to (XIIB), (IC) to (XIIC), (XIII) to (XXIV), (XXV) to (XXXVI), (XXXVII) to (XLVIII), and (XLIX) to (LX), as shown above, and wherein the vasospasms or ischemia are due to a cause selected from the group consisting of angioplasty, vascular graft, stent insertion, and arterial surgery. In preferred embodiments, the sequence modified CGRP is structure V.

The present invention still additionally provides a method for counteracting male impotence or female sexual arousal disorder comprising: applying topically to the penis of a male mammal or to the genital area of a female mammal a pharmaceutical preparation of sequence modified calcitonin gene-related peptide (CGRP) which has been chemically bound to a naturally occurring polyunsaturated fatty acid to make it hydrophobic and skin-penetrable, wherein the sequence modified calcitonin gene related peptide (CGRP) is selected from the group consisting of structures (I) to (XII)), (IA) to (XIIA), (IB) to (XIIB), (IC) to (XIIC), (XIII) to (XXIV), (XXV) to (XXXVI), (XXXVII) to (XLVIII), and (XLIX) to (LX), as shown above. In preferred embodiments, the sequence modified CGRP is structure V.

The present invention yet additionally provides methods of treatment comprising: administering a pharmaceutical preparation of sequence modified calcitonin gene-related peptide to the mammal in a bolus dose of about between 0.25-1 nmol, the pharmaceutical preparation further comprising a free radical scavenger,

wherein the of sequence modified calcitonin gene-related peptide is selected from the group consisting of structures (I) to (XII), (IA) to (XIIA), (IB) to (XIIB), (IC) to (XIIC), (XIII) to (XXIV), (XXV) to (XXXVI), (XXXVII) to (XLVIII), and (XLIX) to (LX), as shown above. In preferred embodiments, the sequence modified CGRP is structure V.

The present invention yet additionally provides methods of treatment comprising: administering a pharmaceutical preparation of sequence modified calcitonin gene-related peptide to the mammal in a bolus dose of about between 0.25-1 nmol, wherein the sequence modified calcitonin gene related peptide (CGRP) is selected from the group consisting of structures (I) to (XII)), (IA) to (XIIA), (IB) to (XIIB), (IC) to (XIIC), (XIII) to (XXIV), (XXV) to (XXXVI), (XXXVII) to (XLVIII), and (XLIX) to (LX), as shown above. In preferred embodiments, the sequence modified CGRP is structure V.

The present invention yet additionally provides a method for counteracting vasospasms in target arteries comprising a specialized vascular bed of a mammal comprising administering a pharmaceutical preparation of sequence modified calcitonin gene-related peptide (CGRP) to the mammal in a bolus dose of between about 0.25-1 nmol to counteract the vasospasm, wherein the sequence modified CGRP is administered locally to the target arteries through a catheter, and wherein the vasospasms comprise occlusion or reocclusion of arteries in a specialized vascular bed, or in acute renal failure, wherein the sequence modified calcitonin gene related peptide (CGRP) is selected from the group consisting of structures (I) to (XII)), (IA) to (XIIA), (IB) to (XIIB), (IC) to (XIIC), (XIII) to (XXIV), (XXV) to (XXXVI), (XXXVII) to (XLVIII), and (XLIX) to (LX), as shown above. In preferred embodiments, the sequence modified CGRP is structure V.

The present invention also additionally provides a method for counteracting vasospasms in target arteries of a mammal comprising administering a pharmaceutical preparation of sequence modified calcitonin gene-related peptide (CGRP) to the mammal in an amount effective to counteract the vasospasm, wherein the vasospasms comprise occlusion or reocclusion of arteries in a specialized vascular bed, or in acute renal failure; wherein the sequence modified CGRP is administered by venous infusion; and the amount of the sequence modified CGRP comprises a dose of about 0.52-10 pmole/kg/min or about 50 to about 200 pmol/kg/hr; and wherein the sequence modified calcitonin gene related peptide (CGRP) is selected from the group consisting of structures (I) to (XII)), (IA) to (XIIA), (IB) to (XIIB), (IC) to (XIIC), (XIII) to (XXIV), (XXV) to (XXXVI), (XXXVII) to (XLVIII), and (XLIX) to (LX), as shown above. In preferred embodiments, the sequence modified CGRP is structure V.

Additional objects, advantages, and novel features of this invention shall be set forth in part in the description and examples that follow, and in part will become apparent to those skilled in the art upon examination of the following or may be learned by the practice of the invention. The objects and the advantages of the invention may be realized and attained by means of the instrumentalities and in combinations particularly pointed out in the appended claims.

### BRIEF DESCRIPTION OF THE FIGURES

FIG. 1 illustrates percent change in systolic, diastolic, mean arterial pressure and heart rate from baseline during infusion of CGRP and analogs in a sedated non-human primate.

FIG. 2 illustrates the hypothesis of CGRP receptor mediated biological responses.

FIG. 3 illustrates the dose response effect of human alpha CGRP on CRE-luciferase activity.

FIG. 4 illustrates the dose response effect of Analog III on CRE-luciferase activity.

FIG. 5 illustrates the dose response effect of Analog V on CRE-luciferase activity.

FIG. 6 illustrates the dose response effect of Analog IX on CRE-luciferase activity.

FIG. 7 illustrates the effect of human alpha and beta CGRP and CGRP analogs III, V and IX on SRE-luciferase activity. VG=VasoGenix.

FIG. 8 illustrates the effect of human alpha and beta CGRP and CGRP analogs III, V and IX on CRE-luciferase activity. VG=VasoGenix.

FIG. 9 illustrates the effect of human alpha and beta CGRP and CGRP analogs on Ramp3-mediated CRE-luciferase activity. VG=VasoGenix.

FIG. 10 illustrates percent change in Systolic BP and HR for alpha CGRP, beta CGRP and VSXP325 (Peptide V) during 30 minute infusions, 1 hour washout between dosing, two separate days, in two non-sedated, telemeterized non-human primates with VAPs at 0.024 ug/kg/min.

FIG. 11 illustrates percent change in mean BP and HR for alpha CGRP, beta CGRP and VSXP325 (Peptide V) during 30 minute infusions, 1 hour washout between dosing, two separate days, in two non-sedated, telemeterized non-human primates with VAPs at 0.024 ug/kg/min.

FIG. 12 illustrates percent change in Systolic BP and HR for alpha CGRP, beta CGRP and VSXP325 (Peptide V) during 1 hour infusions at T30 and T60 minutes in two non-sedated, telemeterized non-human primates with VAPs at 0.024 ug/kg/min.

FIG. 13 illustrates percent change in mean BP and HR for alpha CGRP, beta CGRP and VSXP325 (Peptide V) during 1 hour infusions at T30 and T60 minutes in two non-sedated, telemeterized non-human primates with VAPs at 0.024 ug/kg/min.

### DETAILED DESCRIPTION OF THE INVENTION

### Definitions

The term “biodegradable” means that the polymer matrix will degrade over time, for example by the action of enzymes, by hydrolytic action and/or by other similar mechanisms in the patient's body. By “bioerodible”, it is meant that the polymer matrix will erode or degrade over time due, at least in part, to contact with substances found in the surrounding tissue fluids or cellular action. By “bioabsorbable” it is meant that the polymer matrix will be broken down and absorbed within the human body, for example, by a cell or tissue. “Biocompatible” means that the polymer, the solvent and the resulting polymer matrix will not elicit an adverse biologic response in the patient.

The term “cardiac index” (CI) refers to amount of blood pumped by the heart per minute per meter squared of body surface area.

The term “cardiac output” (CO) refers to the volume of blood pumped by the heart in one minute. Increased cardiac output can indicate a high circulating volume. Decreased cardiac output indicates a decrease in circulating volume or a decrease in the strength of ventricular contraction.

The terms, “cells”, “cell cultures”, “Recombinant host cells”, “host cells”, and other such terms denote, for example, microorganisms, insect cells, and mammalian cells, that can be, or have been, used as recipients for nucleic acid constructs or expression cassettes, and include the progeny of the original cell which has been transformed. It is understood that the progeny of a single parental cell may not necessarily be completely identical in morphology or in genomic or total DNA complement as the original parent, due to natural, accidental, or deliberate mutation. Many cells are available from ATCC and commercial sources. Many mammalian cell lines are known in the art and include, but are not limited to, Chinese hamster ovary (CHO) cells, HeLa cells, baby hamster kidney (BHK) cells, monkey kidney cells (COS), and human hepatocellular carcinoma cells (e.g., Hep G2). Many prokaryotic cells are known in the art and include, but are not limited to, Escherichia coli and Salmonella typhimurium. [Sambrook and Russell, Molecular Cloning: A Laboratory Manual, 3rd edition (Jan. 15, 2001) Cold Spring Harbor Laboratory Press, ISBN: 0879695765]. Many insect cells are known in the art and include, but are not limited to, silkworm cells and mosquito cells. [Franke and Hruby, J. Gen. Virol., 66:2761 (1985); Marumoto et al., J. Gen. Virol., 68:2599 (1987)].

The term “central venous pressure” (CVP) refers to readings that are used to approximate the Right Ventricular End Diastolic Pressure (RVEDP). The RVEDP assesses right ventricular function and general fluid status. Low CVP values typically reflect hypovolemia or decreased venous return, and high CVP values reflect overhydration, increased venous return or right-sided cardiac failure.

An amino acid, as is well known in the art, is an organic acid also bearing an amino group. As used herein, the term refers to alpha-amino acids, wherein the carboxyl group and the amino group are bonded to the same carbon atom, termed the alpha-carbon. Amino acids having chiral alpha-carbon atoms exist in enantiomeric forms. The L-form is the natural, or ribosomal form, and the D-form is the enantiomer. Almost all of the L-amino acids, as are found in naturally occurring proteins, are of the (S) absolute configuration at the α carbon, although L-cysteine is of the (R) absolute configuration, and glycine is achiral.[9].

Peptides, as the term is used herein, refers to polymers of amino acids, each constituent amino acid can referred to as an amino acid residue or simply an amino acid, it being understood that when an amino acid is referred to in the context of a peptide, an amino acid bonded to one or two other groups is referred to. Peptides, as the term is used herein, refers to normal, as well as to retro, inverso, and retro-inverso peptides as defined below, as well as to end-modified forms and sidechain modified forms thereof.

Unless otherwise indicated, peptide sequences are presented in the amino-terminus to carboxyl-terminus direction. For example, H-α-CGRP, as written in the single-letter amino acid code as is well known in the art, is depicted as: ACDTATCVTHRLAGLLSRSGGVVKNNFVPTNVGSKAF-NH₂ (SEQ ID NO:1); this signifies that the A (alanine) occupies the amino- or N-terminal position, and is otherwise unsubstituted, bearing a free amino group, and that F (phenylalanine) occupies the carboxy- or C-terminal position, bearing a modified C-terminal carboxyl group C-terminal carboxmide derivative. The unmodified primary sequence corresponding to this peptide would thus be depicted as ACDTATCVTHRLAGLLSRSGGVVKNNFVPTNVGSKAF-OH (SEQ ID NO:1). Thus, the C-terminal —NH₂ refers to the carboxamide derivative of the sequence. It is understood that in the art, the sequence is sometimes also depicted as: NH₂-ACDTATCVTHRLAGLLSRSGGVVKNNFVPTNVGSKAF-NH₂ (SEQ ID NO:1); in this manner of depiction, the first amino group does not refer to a derivative, but only to the identity of the alanine A as being in the amino terminal position. To avoid any ambiguity, the convention used herein does not show an amino terminal —NH₂ to identify that residue as being amino-terminal, as that is understood from the position of the amino acid residue in the sequence.

Any end modifications of a sequence depicted in the manner used herein can be shown as a chemical substructure, which is understood to be bonded to an NH group on the amino terminal end of the peptide, and to a carbonyl group on the carboxy terminal end of the peptide. For example, an N-terminal acetylated CGRP peptide can be depicted as: CH₃C(═O)-ACDTATCVTHRLAGLLSRSGGVVKNNFVPTNVGSKAF-NH₂ (SEQ ID NO:1), or, using the standard abbreviation of Ac as signifying an acetyl group, as: Ac-ACDTATCVTHRLAGLLSRSGGVVKNNFVPTNVGSKAF-NH₂ (SEQ ID NO:1), wherein it is understood that the acetyl group forms an amide bond with alpha-amino group of the N-terminal amino acid residue.

“CGRP”, such as “native CGRP”, as the terms are used herein refers to “calcitonin gene-related peptide” in one of its isoforms, such as the α or β form, as isolated from a natural source or prepared from a cloned nucleic acid comprising a coding sequence for an isoform of the peptide as it occurs naturally. For example H-α-CGRP refers to the α isoform of CGRP as isolated from a human source. Some known isoforms of CGRP are discussed above. The term “CGRP” or “native CGRP” refers to a structure, not to the source, so a native CGRP can be a chemically synthesized material, or can be obtained from expression of a cloned gene, or can be isolated from the tissue of an organism.

A “normal” peptide is a peptide composed of L-amino acid residues, such as native CGRP, but need not have a native sequence. A normal peptide is a peptide composed of L-amino acid residues wherein the sequence includes substitutions of amino acids at one or more positions in the peptide with respect to a native form.

A “modified CGRP,” “modified peptide,” “sequence modified CGRP” or “CGRP analog” as the term is used herein is an analog of a native isoform from a source such as human wherein some modification(s) of the native structure has been made. The terms refer to a peptide or a peptide analog wherein a structural similarity exists, such as in primary sequence, in three-dimensional conformation in solution, or in spatial disposition of pharmacophores or functional groups involved in binding interactions with macromolecules. A modified or sequence-modified CGRP or CGRP analog can be a normal peptide wherein one or more amino acid substitutions have been made, or wherein a portion or portions of the sequence are deleted, or wherein additional amino acid residues have been added either at a terminus or internally or both; or it can be a retro form, an inverso form, or a retro-inverso form as defined below, in complete or partial form, or a hybrid form as defined below; or it can be any of the above wherein modifications of end groups (C-terminus and N-terminus) or sidechains has been made.

A “retro CGRP” or “retro form” is a sequence composed of L-amino acid residues, but having a reversed sequence with respect to the normal or native CGRP isoform to which it corresponds. A compound of the invention can include both normal and retro domains within a single molecule. Retro forms can be synthesized chemically using the techniques typically used for peptide synthesis, such as solid phase synthesis, but programming the opposite amino acid sequence for a completely retro form, or a sequence incorporating the retro domains for a partially retro form. A retro form can also be obtained by ribosomal expression of an appropriately reversed sequence of codons in a nucleic acid sequence in an expression vector. A retro form can be “complete,” in which the entire sequence is reversed, or “partial,” wherein only a portion of the sequence is reversed.

An “inverso CGRP” or “inverso form” is a peptide composed of D-amino acid residues, i.e., the enantiomers of L-amino acid residues, having the same sequence as the normal or native peptide isoform to which it corresponds. A glycine residue, being achiral, is not altered in an inverso form. A compound of the invention can include both normal and inverso domains within a single molecule. Retro forms can be synthesized chemically using the techniques typically used for peptide synthesis, such as solid phase synthesis, only using amino acid reagents prepared from the readily available D-forms of the appropriate amino acids. Coupling and deblocking chemistries are unaltered from normal peptide chemical synthesis. An inverso form can be “complete,” in which the entire sequence is composed of D-amino acid residues, or “partial,” wherein only a portion of the sequence is composed of D-amino acid residues and the remainder is composed of L-amino acid residues. As used herein, a peptide composed of D-amino acid residues is depicted as the sequence, N-terminal to C-terminal, followed by the designation “all-D” when all residues (except glycine) are the D form of the amino acid, or “(Xₘ-Xₙ)-D” when residues number m through n from the N-terminal residue are the D form.

A “retro-inverso CGRP” or “retro-inverso form” is a peptide composed of D-amino acids and having a reversed sequence with respect to the normal or native peptide to which it corresponds. A compound of the invention can include both normal and retro-inverso domains within a single molecule. Retro-inverso forms can be synthesized chemically using the techniques typically used for peptide synthesis, such as solid phase synthesis, only using amino acid reagents prepared from the readily available D-forms of the appropriate amino acids, and programming a reverse order of assembly. Coupling and deblocking chemistries are unaltered from normal peptide chemical synthesis. A retro-inverso form can be “complete,” in which the entire sequence is composed of D-amino acid residues and is sequence-reversed, or “partial,” wherein only a portion of the sequence is composed of D-amino acid residues and the remainder is composed of L-amino acid residues, or only a portion of the sequence is reversed, or both. As used herein, a peptide composed of D-amino acid residues is depicted as the sequence, N-terminal to C-terminal, followed by the designation “all-D” when all residues (except glycine) are the D form of the amino acid, or “(Xₘ-Xₙ)-D” when residues number m through n from the N-terminal residue are the D form. Thus, a complete retro-inverso form has the reverse sequence with respect to the reference sequence, and is “all-D”.

In any peptide formula or depiction, if there is no designation of any amino acid residues as being of the D-form in the manner described above, it is understood that the chiral amino acids are all of the L-form.

One rationale behind the use of retro-inverso peptides is that the retro-inverso peptide is believed to approximate the same tertiary structure or conformation in solution as does the normal peptide to which it corresponds, but it will be largely impervious to the action of endogenous peptide-degrading enzymes present in the mammalian body, which act largely on peptides composed of L-amino acid residues. In this way, a molecule can be obtained that will have a substantial half-life in the body, but will exert a similar biological effect as the corresponding normal peptide. See, for example, M. D. Fletcher and M. M. Campbell (1998), “Partially Modified Retro-Inverso Peptides: Development, Synthesis, and Conformational Behavior,” Chem. Rev, 98, 763-795.

A compound of the invention can include any combination of normal, retro, inverso, and retro-inverso domains within a single molecule, which is termed a “hybrid” form. Any such compounds are readily prepared using standard solid phase peptide synthesis techniques. For example, appropriate N-protected D- or L-amino acid reagents using Fmoc or t-Boc N-protection can be coupled using standard carboxyl activation chemistries, as are well known in the art.

Compounds of the invention also include any of the above normal, retro, inverso, retro-inverso, or hybrid forms wherein modifications of the termini, i.e., the amino terminus (N-terminus) and the carboxyl terminus (C-terminus) have been made. For example, a carboxyl terminus can be prepared in a carboxamide (CONH₂) form, as is well known in the art. An amino terminal NH₂ group can be acylated, for example with acetyl, or with long chain fatty acyl, or by aroyl groups such as benzoyl. Specific examples of N-acylated forms include formyl, acetyl, (C₁₂-C₁₈) fatty acyl, and benzoyl forms. N-acylated forms, being more lipophilic than the underivatized form, can be effective in penetration of biological barriers such as skin, blood-brain barrier, and the like.

Or, N- and C-terminal residues, reversed in a retro-inverso form can be mimicked to provide that even in a retro or a retrao-inverso form, the amino- and carboxy-terminal residues bear an amino or a carboxyl functionality, despite being at the opposite end of the sequence. For example, a carboxyl terminus can be converted into a pseudo amino terminus by use of a diamino functionality, such as gem-diamino ethane mimicking an alanine residue. A mono-amide is formed by coupling the carboxyl group of the C-terminus with one of the amino groups of the gem-diaminoethane, such that the remaining free amino group mimics a peptide amino terminus. Similarly, an amino terminus can be converted into a pseudo-carboxyl terminus by use of a dicarboxyl functionality, such as a malonic acid species, for example benzyl malonic acid mimicking a phenylalanine residue by formation of a mono-amide of the malonate with the amino group of the N-terminus, or the malonate bonded by one carboxyl to the N-terminal amino group, the other carboxyl being in the form of a carboxamide, mimicking a C-terminal carboxamide, as is present in native CGRP. See, for example, M. D. Fletcher and M. M. Campbell (1998), “Partially Modified Retro-Inverso Peptides Development, Synthesis, and Conformational Behavior,” Chem. Rev., 98, 763-795. In this way, a retro or retro-inverso form, which normally would have the identities of the amino acid residues at the amino and carboxyl termini reversed, can be prepared in a form where the structures at the amino or carboxyl termini are structurally analogous to those in the normal or native form. When a peptide identified as “all-D” has terminal modification groups attached thereto, it is understood that the designation of chirality does not apply to the terminal modification groups, which if chiral may be of either absolute configuration, or a mixture thereof.

The residues of any of the above peptide forms can be bonded by amide bonds, as in the case of a normal peptide, or can be further modified to include pseudo-peptide (w) bonds such as ester, ketomethylene, or sulfonamido bonds, and the like, as are well known in the art. Such substitution of pseudo-peptide bonds for amide bonds can further increase the resistance of the retro-inverso peptide to proteolytic degradation in the mammalian body.

The amino acid residues of any of the above peptide forms can be further modified by derivatization of one or more side chains; such as esterification of carboxyl side chains, acylation of amino side chains, and the like, as are well known in the art.

In particular, a sequence modified CGRP peptide of the invention can include normal sequence and retro-inverso sequence segments. Such a molecule is termed a “partial retro-inverso form”. For example, a partial retro-inverso form can be a peptide analog wherein the entire sequence is in the retro order with respect to a native CGRP isoform, and is composed only of D-amino acid residues, except the C- and N-terminal amino acids are the same C- and N-terminal L-amino acids found in the native CGRP isoform. Or, a partial retro-inverso form can include interspersed domains or segments of D-amino acid residues and L-amino acid residues, wherein some are in the normal sequence with respect to the corresponding native CGRP or normal sequence modified form thereof, and some are in retro sequence. Such a hybrid form can include domains composed of any of the above-described types.

Compounds of the invention can act either as “agonists” or “antagonists” of receptors for CGRP ligands, as are well known in the art. An “agonist” is a compound that binds to a receptor and activates the receptor, whereas an “antagonist” is a compound that binds to a receptor but does not activate the receptor, and can block agonists from binding to and activating the receptor.

The term “change in heart rate” refers to a condition that indicates tachycardia or increased workload.

The term “dyspnea” means shortness of breath. Dyspnea is a primary clinical endpoint to address efficacy in heart failure treatments.

As used herein, “flowable” refers to the ability of the composition to be administered by any suitable means into the body of a patient. For example, the composition can be injected into a specific site in the patient with the use of a syringe and puncture needle or placed into accessible tissue sites through a cannula. The ability of the composition to be injected into a patient will typically depend upon the viscosity of the composition. The composition will therefore have a suitable viscosity such that the composition can be forced through the medium (e.g., syringe) into the body of a patient.

The term “gel” refers a substance having a gelatinous, jelly-like, or colloidal property (Id., p. 567).

The term “hemodynamic functions” includes, but is not limited to, heart rate, right atrial pressure, pulmonary artery pressure, pulmonary artery wedge pressure, systemic arterial pressure, cardiac output (i.e., cardiac index), stroke volume index, pulmonary vascular resistance, and systemic vascular resistance.

The term “improved hemodynamic functions” includes, but is not limited to, increased cardiac output, decreased pulmonary artery wedge pressure, decreased pulmonary vascular resistance, and decreased systemic vascular resistance, increased cardiac contractility, normal diastolic compliance, increased stroke volume and reduced pulmonary congestion.

An “inclusion body” is an amorphous polypeptide deposit in the cytoplasm of a cell. In general, inclusion bodies comprise aggregated protein that is improperly folded or inappropriately processed.

An “inclusion body leader partner” is a peptide that causes a polypeptide to which it is attached to form an inclusion body when expressed within a bacterial cell. The inclusion body leader partners of the invention can be altered to confer isolation enhancement onto an inclusion body that contains the altered inclusion body leader partner.

The term “isolated” when used in relation to a nucleic acid, as in “isolated oligonucleotide” or “isolated polynucleotide” refers to a nucleic acid sequence that is identified and separated from at least one contaminant with which it is ordinarily associated in its source. Thus, an isolated nucleic acid is present in a form or setting that is different from that in which it is found in nature. In contrast, non-isolated nucleic acids (e.g., DNA and RNA) are found in the state they exist in nature. For example, a given DNA sequence (e.g., a gene) is found on the host cell chromosome in proximity to neighboring genes; RNA sequences (e.g., a specific mRNA sequence encoding a specific protein), are found in the cell as a mixture with numerous other mRNAs that encode a multitude of proteins. However, isolated nucleic acid includes, by way of example, such nucleic acid in cells ordinarily expressing that nucleic acid where the nucleic acid is in a chromosomal location different from that of natural cells, or is otherwise flanked by a different nucleic acid sequence than that found in nature. The isolated nucleic acid or oligonucleotide may be present in single stranded or double stranded form. When an isolated nucleic acid or oligonucleotide is to be utilized to express a protein, the oligonucleotide contains at a minimum, the sense or coding strand (i.e., the oligonucleotide may single stranded), but may contain both the sense and anti sense strands (i.e., the oligonucleotide may be double stranded).

The term “isolated” when used in relation to a polypeptide, as in “isolated protein” or “isolated polypeptide” refers to a polypeptide that is identified and separated from at least one contaminant with which it is ordinarily associated in its source. Thus, an isolated polypeptide is present in a form or setting that is different from that in which it is found in nature. In contrast, non-isolated polypeptides (e.g., proteins and enzymes) are found in the state they exist in nature.

The term “left ventricular stroke index” (LVSI) refers to the difference in contractile position of the left ventricle from the resting position to the point of maximum contraction.

As used herein, a “liquid” is a substance that undergoes continuous deformation under a shearing stress (Concise Chemical and Technical Dictionary, 4^{th} Enlarged Ed., Chemical Publishing Co., Inc., p. 707, New York, N.Y. (1986)).

The term “mean arterial pressure” (MAP) refers to changes in the relationship between cardiac output (CO) and systemic vascular resistance (SVR) and reflects the arterial pressure in the vessels perfusing the organs. A low MAP indicates decreased blood flow through the organs, and a high MAP indicates an increased cardiac workload.

The term “neurohormone release” refers to a response by the kidneys to increase renal blood flow by releasing the vasoconstricting neurohormones norepinephrine, epinephrine, and rennin. These hormones act to constrict peripheral vasculature adversely affecting the PVR.

For purposes of this invention, a “patient having HF” refers to a person having Stage B, Stage C, or Stage D heart failure as classified in the American College of Cardiology Guidelines for the Evaluation and Management of Chronic Heart Failure in the Adult. While the American College of Cardiology Guidelines excluded HF in children, for purposes of this invention the methods are to be considered applicable to any patient, regardless of age.

The term “preload” refers to the combination of pulmonary blood filling the atria and the stretching of myocardial fibers. Preload is regulated by the variability in intravascular volume. A reduction in volume decreases preload, whereas an increase in volume increases preload, mean arterial pressure (MP) and stroke index (SI). Preload occurs during diastole.

“Promoter” refers to a nucleotide sequence, usually upstream (5′) to its coding sequence, which controls the expression of the coding sequence by providing the recognition for RNA polymerase and other factors required for proper transcription. “Promoter” includes a minimal promoter that is a short DNA sequence comprised of a TATA-box and other sequences that serve to specify the site of transcription initiation, to which regulatory elements are added for control of expression. “Promoter” also refers to a nucleotide sequence that includes a minimal promoter plus regulatory elements that is capable of controlling the expression of a coding sequence. Promoters may be derived in their entirety from a native gene, or be composed of different elements derived from different promoters found in nature, or even be comprised of synthetic DNA segments. A promoter may also contain DNA segments that are involved in the binding of protein factors that control the effectiveness of transcription initiation in response to physiological or environmental conditions.

The term “pulmonary artery pressure” (PA pressure) refers to blood pressure in the pulmonary artery. Increased pulmonary artery pressure may indicate a left-to-right cardiac shunt, pulmonary artery hypertension, COPD, emphysema, pulmonary embolus, pulmonary edema, or left ventricular failure.

The term “pulmonary capillary wedge pressure” (PCWP or PAWP) refers to a pressure are used to approximate LVEDP (left ventricular end diastolic pressure). High PCWP may indicate left ventricle failure, mitral valve pathology, cardiac insufficiency, and/or cardiac compression post hemorrhage. PCWP is a primary clinical endpoint to address efficacy in heart failure treatments.

The term “pulmonary vascular resistance” (PVR) refers to the measurement of resistance or the impediment of the pulmonary vascular bed to blood flow. An increased PVR is caused by pulmonary vascular disease, pulmonary embolism, pulmonary vasculitis, or hypoxia. A decreased PVR is caused by medications such as calcium channel blockers, aminophylline or isoproterenol, or by the delivery of O₂.

The term “purified” or “to purify” means the result of any process that removes some of a contaminant from the component of interest, such as a protein or nucleic acid. The percent of a purified component is thereby increased in the sample.

As used herein, “pure” means an object species is the predominant species present (i.e., on a molar basis it is more abundant than any other individual species in the composition), and preferably a substantially purified fraction is a composition wherein the object species comprises at least about 50 percent (on a molar basis) of all macromolecular species present. Generally, a “substantially pure” composition will comprise more than about 80 percent of all macromolecular species present in the composition, more preferably more than about 85%, about 90%, about 95%, and about 99%. Most preferably, the object species is purified to essential homogeneity (contaminant species cannot be detected in the composition by conventional detection methods) wherein the composition consists essentially of a single macromolecular species.

“Quality of life” refers to one or more of a person's ability to walk, climb stairs, do errands, work around the house, participate in recreational activities, and/or not requiring frequent rest intermittently during activities, and/or the absence of sleeping problems or shortness of breath.

The term “renal blood flow” (RBF) refers to the measurement of blood flow into the kidneys. Twenty percent of cardiac output passes through the kidneys, which compromise less than 1% of body weight. Increased renal blood flow is proportional to increased renal function and urine output.

The term “renal glomerular filtration” (RGF) refers to the first step in urine formation as protein-free ultrafiltrate plasma crosses the walls of the glomerular capillaries. Increased renal blood flow increases flow of plasma across the glomeruli, increasing urine output.

The term “right ventricular pressure” (RV Pressure) refers to a direct measurement that indicates right ventricular function and general fluid status. High RV pressure may indicate pulmonary hypertension, right ventricle failure, or congestive heart failure.

The terms “stroke index” or “stroke volume index” (SI or SVI) are used interchangeably and refer to the amount of blood ejected from the heart in one cardiac cycle, relative to Body Surface Area (BSA). SVI is measured in milliliters per meter squared per beat. An increased SVI can be indicative of early septic shock, hyperthermia or hypervolemia, or can be caused by medications such as dopamine, Dobutamine or Digitalis. A decreased SVI can be caused by CHF, late septic shock, beta-blockers or an MI.

The term “stroke volume” (SV) refers to the amount of blood pumped by the heart per cardiac cycle, and is measured in milliliters per beat. A decreased SV may indicate impaired cardiac contractility or valve dysfunction and may result in heart failure. An increased SV can be caused by an increase in circulating volume or an increase in inotropy.

The term “systemic vascular resistance” (SVR refers to the measurement of resistance or impediment of the systemic vascular bed to blood flow. An increase in SVR can be caused by vasoconstrictors, hypovolemia or late septic shock. A decrease in SVR can be caused by early septic shock, vasodilators, morphine, nitrates or hypercarbia.

The term “therapeutically effective amount” refers to the amount of sequence modified CGRP and/or other vasoactive agents that are effective to achieve its intended purpose. While individual patient needs may vary, determination of optimal ranges for effective amounts of hydrophobic-sequence modified CGRP and/or other vasoactive agents is within the skill of the art. Generally, the dosage required to provide an effective amount of the compound and/or composition, and which can be adjusted by one of ordinary skill in the art will vary, depending on the age, health, physical condition, weight, extent of the dysfunction of the recipient, frequency of treatment and the nature and scope of the dysfunction.

“Treating HF” as used herein refers to treating any one or more of the conditions underlying HF, including, without limitation, decreased cardiac contractility, abnormal diastolic compliance, reduced stroke volume, pulmonary congestion, decreased cardiac output, and other diminished hemodynamic functions, while minimizing or attenuating deleterious effects that may be associated with the long-term administration of sequence modified CGRP such as nausea, diarrhea, severe facial flushing and intermittent tachycardia. “Treating HF” also includes relieving or attenuating symptoms associated with HF.

A “microgram” (μg) is 1 millionth of a gram, i.e., 10⁻⁶ grams.

A “nanogram” (ng) is 1 billionth of a gram, i.e., 10⁻⁹ grams.

A “picogram” (pg) is 1 trillionth of a gram, i.e., 10⁻¹² grams.

TABLE 1 provides normal values for various hemodynamic parameters.

TABLE 1

| Hemodynamic Parameter | Normal Value |
|---|---|
| Blood Pressure Systolic (SBP) | 90-140 mmHg |
| Diastolic (DBP) | 60-90 mmHg |
| Mean Arterial Pressure (MAP) | 70-100 mmHg |
| Cardiac Index (CI) | 2.5-4 L/min/m² |
| Cardiac Output (CO) | 4-8 L/min |
| Central Venous Pressure (CVP) | 2-6 mmHg |
| Right Ventricular Pressure (RV) | Systolic-20-30 mmHg |
| | Diastolic 0-5 mmHg |
| Stroke Index (SI) | 25-45 mL/m² |
| Pulmonary Artery Pressure (PA) | Systolic: 20-30 mmHg (PAS) |
| | Diastolic: 8-12 mmHg (PAD) |
| | Mean: 25 mmHg (PAM) |
| Pulmonary Capillary Wedge | 4-12 mmHg (PWCP) |
| Pressure | |
| Pulmonary Vascular Resistance | 37-250 dynes/sec/cm³. |
| (PVR) | |
| Systemic Vascular Resistance | 800-1200 dynes/sec/ |
| (SVR) | |

All amino acid residues identified herein are in the natural L configuration unless otherwise specified, for example in the section describing retro-inverso forms of CGRP, below. In keeping with standard polypeptide nomenclature, abbreviations for amino acid residues are as shown in the following Table of Correspondence.

TABLE 2
TABLE OF CORRESPONDENCE

| 1 Letter Code | 3 Letter Code | Amino Acid |
|---|---|---|
| Y | Tyr | tyrosine |
| G | Gly | glycine |
| F | Phe | phenylalanine |
| M | Met | methionine |
| A | Ala | alanine |
| S | Ser | serine |
| I | Ile | isoleucine |
| L | Leu | leucine |
| T | Thr | threonine |
| V | Val | valine |
| P | Pro | proline |
| K | Lys | lysine |
| H | His | histidine |
| Q | Gln | glutamine |
| E | Glu | glutamic acid |
| W | Trp | tryptophan |
| R | Arg | arginine |
| D | Asp | aspartic acid |
| N | Asn | asparagine |
| C | Cys | cysteine |

### Sequence Modified CGRP

As noted above, amino acid sequence variation between human alpha CGRP (α-CGRP) and beta CGRP (β-CGRP) can be found at the 3, 22 and 25 positions.

The alpha form differs from the beta form by substitution of aspartic acid (D) for asparagine (N) at position 3; valine (V) for methionine (M) at position 22; and asparagine (N) for serine (S) at position 25. In the alpha form below the aspartic acid (D), valine (V) and asparagine (N) are highlighted (bold and underlined). In the beta form listed below asparagine (N), methionine(M) and serine (S) are highlighted (bold and underlined). All peptide structures are drawn with the amino terminal amino acid residue on the left-hand side of the molecule, the additional hydrogen atom being implicitly understood, and the phenylalanine carboxamide [—C(═O)—NH₂] denoted as F—NH₂ on the right-hand side of the molecule.

Human α-CGRP: Net positive charge of +4
(SEQ ID NO: 1)
A C D T A T C V T H R L A G L L S R S G G V V K N
N F V P T N V G S K A F-NH₂
Human β-CGRP: Net positive charge of +5
(SEQ ID NO: 2)
A C N T A T C V T H R L A G L L S R S G G M V K S
N F V P T N V G S K A F-NH₂

The bold letters at positions 3, 22 and 25 indicate the amino acid residues where variations exist between human α-CGRP and human β-CGRP. The underlined letters represent human α-CGRP residues that are substituted with human β-CGRP.

Peptide function is known to be largely dependent upon the structure and organization of the component amino acid residues. The aspartic acid, valine and asparagine residues at positions 3, 22 and 25 respectively in the alpha form and the asparagine, methionine, and serine at positions 3, 22 and 25 respectively in the beta form of CGRP act in concert with one another and with the remaining amino acids to provide the specific activity and function of each molecule. It is also known that synthetic modifications of the C and N terminal portions of the peptides result in substantial loss of activity and potency and amino acid sequence 1-7, the cyclic disulphide sequence, activates the receptor. Amino acid sequence 8-37 has high binding affinity with the receptor sites and is a CGRP antagonist. Tryptic and chymotryptic produced fragments as well as modifications in the disulphide bridge lead to loss of activity (see Zaidi et al., Biochem J. 1990 Aug. 1; 269(3):775-80, the disclosure of which is incorporated herein by reference in its entirety).

Substitution of single amino acids at positions 3, 22 and 25 leads to three entirely new peptide molecules.

Replacing D of alpha CGRP at position 3 with N provides Modified

CGRP Compound (I) having a net positive charge of +5.

Sequence Modified CGRP compound
(I; SEQ ID NO: 3)
A C N T A T C V T H R L A G L L S R S G G V V K N
N F V P T N V G S K A F-NH₂

Replacing V of alpha CGRP at position 22 with M provides Modified CGRP Compound (II) having a net positive charge of +4.

Sequence Modified CGRP Compound
(II; SEQ ID NO: 4)
A C D T A T C V T H R L A G L L S R S G G M V K N
N F V P I N V G S K A F-NH₂

Replacing N of alpha CGRP at position 25 with S provides Modified CGRP Compound (III) having a net positive charge of +4.

Sequence Modified CGRP Compound
(III: SEQ ID NO: 5)
A C D T A T C V T H R L A G L L S R S G G V V K S
N F V P T N V G S K A F-NH₂

Replacing D of alpha CGRP at position 3 with N; and V at position 22 with M would provide Modified CGRP Compound (IV) having a net positive charge of +5.

Sequence Modified CGRP Compound
(IV; SEQ ID NO: 6)
A C N T A T C V T H R L A G L L S R S G G M V K N
N F V P T N V G S K A F-NH₂

Replacing D of alpha CGRP at position 3 with N; and N at position 25 with S would provide Sequence Modified CGRP Compound (V) having a net positive charge of +5.

Sequence Modified CGRP Compound
(V; SEQ ID NO: 7)
A C N T A T C V T H R L A G L L S R S G G V V K S
N F V P T N V G S K A F-NH₂

Replacing V of alpha CGRP at position 22 with M; and N at position 25 with S would provide Sequence Modified CGRP Compound (VI) having a net positive charge of +4.

Sequence Modified CGRP Compound
(VI; SEQ ID NO: 8)
A C D T A T C V T H R L A G L L S R S G G M V K S
N F V P T N V G S K A F-NH₂

Chicken CGRP has aspartic acid (D) at position 14. A human synthetic analogue with an aspartic acid (D) at position 14 is a more potent producer of a cyclic AMP response in a preosteoblast cell line (KS-4) than human a CGRP. Thus, the glycine (G) residue at position 14 in all six of the above analogues was replaced with aspartic acid (D). The amino acid sequence for each of these compounds is shown below as Sequence Modified CGRP Compounds (VII-XII).

The bold letters at positions 3, 22 and 25 indicate the amino acid residues where variations exist between human α-CGRP and human β-CGRP. A bold letter “D” at position 14 indicates aspartic acid from chicken CGRP. The underlined letters represent human α-CGRP residues that are substituted with human β-CGRP and/or chicken CGRP residues.

Sequence Modified CGRP Compound
(VII; SEQ ID NO: 9)
A C N T A T C V T H R L A D L L S R S G G V V K N
N F V P T N V G S K A F-NH₂
Sequence Modified CGRP Compound
(VIII; SEQ ID NO: 10)
A C D T A T C V T H R L A D L L S R S G G M V K N
N F V P T N V G S K A F-NH₂
Sequence Modified CGRP Compound
(IX; SEQ ID NO: 11)
A C D T A T C V T H R L A D L L S R S G G V V K S
N F V P T N V G S K A F-NH₂
Sequence Modified CGRP Compound
(X; SEQ ID NO: 12)
A C N T A T C V T H R L A D L L S R S G G M V K N
N F V P T N V G S K A F-NH₂
Sequence Modified CGRP Compound
(XI; SEQ ID NO: 13)
A C N T A T C V T H R L A D L L S R S G G V V K S
N F V P T N V G S K A F-NH₂
Sequence Modified CGRP Compound
(XII; SEQ ID NO: 14)
A C D T A T C V T H R L A D L L S R S G G M V K S
N F V P T N V G S K A F-NH₂

In some embodiments, the sequence modified CGRP compounds (I-XII) above are contemplated to have an ester, such as an ethyl ester, in place of the C-terminal amide shown. In some embodiments, the sequence modified CGRP is bis-acetylated or N-acetylated (see Zaidi et al., supra).

In some embodiments, a retro, inverso, or retro-inverso sequence modified CGRP compound is provided. As defined above, a retro-inverso sequence is determined by the sequence of the normal peptide, which contains L-amino acids in a defined N→C sequence. The retro-inverso peptide contains D-amino acids in the opposite sequence; i.e., the N-terminal amino acid of the normal peptide is the C-terminal amino acid of the retro-inverso peptide, and the C-terminal amino acid of the normal peptide is the N-terminal amino acid of the retro-inverso peptide. In Table 3, a series of modified CGRP molecules of the normal peptide type are shown with the site-specific amino acid residue substitutions for each species. In the corresponding retro-inverso form, as indicated in the right hand column, the mutation is made at the corresponding position, bearing in mind that N-terminus and C-terminus are switched in the retro-inverso form, so numbering is reversed as well. The retro-inverso form also uses the corresponding D-amino acid, e.g., the retro-inverso form uses D-alanine if an L-alanine residue is present in the corresponding (reversed) position of the normal peptide. Glycine, being achiral, has no chiral forms.

TABLE 3
H-α-CGRP and Substitutions at each residue by position;
retro-inverso position shown in righthand column

| Position | Amino Acid | Substitute | Retro-Inverso |
|---|---|---|---|
| 1 | Alanine = A | Glycine = G | 37 |
| | | Isoleucine = I | |
| | | Leucine = L | |
| | | Serine = S | |
| | | Valine = V | |
| 3 | Aspartic Acid = D | Asparagine = N | 35 |
| | | Glutamic Acid = E | |
| | | Glutamine = Q | |
| 4 | Threonine = T | Serine = S | 34 |
| 5 | Alanine = A | Isoleucine = I | 33 |
| | | Glycine = G | |
| | | Leucine = L | |
| | | Valine = V | |
| 6 | Threonine = T | Serine = S | 32 |
| 8 | Valine = V | Isoleucine = I | 30 |
| | | Leucine = L | |
| | | Alanine = A | |
| 9 | Threonine | Serine = S | 29 |
| 12 | Leucine = L | Alanine = A | 26 |
| | | Glycine = G | |
| | | Isoleucine = I | |
| | | Valine = V | |
| 13 | Alanine = A | Glycine = G | 25 |
| | | Isoleucine = I | |
| | | Leucine = L | |
| | | Valine = V | |
| 14 | Glycine = G | Alanine = A | 24 |
| | | Isoleucine = I | |
| | | Leucine = L | |
| | | Valine = V | |
| | | Aspartic Acid = D | |
| | | Glutamic acid = E | |
| 15, 16 | Leucine = L | Alanine = A | 23, 22 |
| | | Glycine = G | |
| | | Isoleucine = I | |
| | | Valine = V | |
| 17, 19 | Serine = S | Threonine = T | 21, 19 |
| 20, 21 | Glycine = G | Alanine = A | 18, 17 |
| | | Isoleucine = I | |
| | | Leucine = L | |
| | | Valine = V | |
| | | Aspartic Acid = D | |
| 22, 23 | Valine = V | Isoleucine = I | 16, 15 |
| | | Leucine = L | |
| | | Alanine = A | |
| | | Methionine = M | |
| 24 | Lysine = 1 | Arginine = R | 14 |
| 25, 26 | Asparagine | Glutamine = Q | 13, 12 |
| | | Aspartic Acid = D | |
| 27 | Phenylalanine = F | Tyrosine = Y | 11 |
| 28 | Valine = V | Isoleucine = I | 10 |
| | | Leucine = L | |
| | | Alanine = A | |
| 30 | Threonine = T | Serine = S | 8 |
| 31 | Asparagine = N | Glutamine = G | 7 |
| 32 | Valine = V | Isoleucine = I | 6 |
| | | Leucine = L | |
| | | Alanine = A | |
| 33 | Glycine = G | Alanine = A | 5 |
| | | Isoleucine = I | |
| | | Leucine = L | |
| | | Valine = V | |
| 34 | Serine = S | Threonine = T | 4 |
| 35 | Lysine = L | Argininne = R | 3 |
| | | Aspartic Acid = D | |
| | | Glutamic acid = E | |
| 36 | Alanine = A | Glycine = G | 2 |
| | | Isoleucine = I | |
| | | Leucine = L | |
| | | Valine = V | |

Also contemplated are conservative amino acid changes in the above CGRP compounds. Although conservative amino acid changes alter the primary sequence of the protein or peptide, they do not normally alter its function. Conservative amino acid substitutions typically include substitutions within the following groups:

glycine, alanine;

valine, isoleucine, leucine;

aspartic acid, glutamic acid;

asparagine, glutamine;

serine, threonine;

lysine, arginine;

phenylalanine, tyrosine.

Modifications (which do not normally alter primary sequence) include in vivo, or in vitro chemical derivatization of polypeptides, e.g., N-terminal or sidechain acylation, such as acetylation, or conversion of a carboxyl group, such as the carboxy-terminal carboxyl group to a carboxamido group. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

CGRP 8-37 peptides with the above-identified substitutions beyond position 7 are also contemplated herein, including substitutions at positions 14, 22 and 25 as in analogs I through XII For example, the following peptide is contemplated: VTHRLAGLLSRSGGVVKNNFVPTNVGSKAF-NH₂ (SEQ ID NO:42), wherein the amino acid at position 14 is glycine, aspartic acid or glutamic acid, the amino acid at position 22 is valine or methionine and the amino acid at position 25 is asparagine or serine. In some embodiments, the R group at position 8 is valine or is in the desvalanyl form where the valine is replaced by a hydrogen substituted for the alpha amine function of the threonine at position 9. The substitutions shown in Table 3 are also contemplated for CGRP 8-37. Retro-inverso CGRP 8-37 peptides are also contemplated herein, as are end-modified forms thereof. CGRP 8-37 peptides are defined as CGRP sequences wherein amino acid residues AA1-AA7 (i.e., the seven N-terminal residues) are deleted. These deletion sequences can also be termed AA1-AA7 deleted sequence modified CGRP peptides. A CGRP peptide lacking the N-terminal alanine residue can also be termed an AA1-desalanyl sequence modified CGRP peptide

For example, the above-identified normal peptide, VTHRLAGLLSRSGGVVKNNFVPTNVGSKAF-NH₂ (SEQ ID NO:42), can be prepared in retro-inverso form composed of D-amino acid residues, the retro-inverso form having the sequence (all D)-FAKSGVNTPVFNNKVVGGSRSLLGALRHTV-NH₂. In a retro-inverso form corresponding to a normal peptide, the numbering of the residues is reversed, as peptides are numbered from their N-terminus, and the N-terminal residue of a corresponding retro-inverso form is the C-terminal residue of the normal form. It is believed that the retro-inverso form assumes a similar solution conformation to the normal peptide form and can thus carry out corresponding metabolic functions as the normal peptide form.

Sequence modified CGRP has significant advantages over conventional drug treatments. First, sequence modified CGRP should not produce the potentially dangerous side effects, toxicity and tolerance associated with conventional cardiovascular drugs such as nitroglycerin, Dobutamine and Natrecor. In fact, sequence modified CGRP should down-regulate immune response via inhibition of cytokine release and should be able to be safely administered to immunosuppressed subjects without the induction of sensitivity. Second, since sequence modified CGRP possesses multiple hemodynamic benefits, it potentially reduces or eliminates the need for drug cocktails to maintain specific hemodynamic functions. Third, more than 20 years of research on the potency, safety and efficacy of the human CGRP in animals and humans have demonstrated the cardiovascular benefits of CGRP and have shown that CGRP exhibits virtually no side effects or tolerance when administered systemically. Sequence modified CGRP will behave similarly. Retro-inverso forms of sequence modified CGRP can also exhibit these advantages, with a further advantage of resistance to proteolytic degradation in a mammalian body due to a lack of susceptibility of the retro-inverso forms to hydrolysis catalyzed by endogenous proteases or peptidases.

Other forms of sequence modified CGRP that are suitable for use in the methods of this invention are pharmaceutically acceptable salts and pharmaceutically acceptable prodrugs of sequence modified CGRP including retro-inverso forms thereof. A “pharmaceutically acceptable prodrug” is a compound that may be converted under physiological conditions or by solvolysis to the specified compound or to a pharmaceutically acceptable salt of such compound. Prodrugs of sequence modified CGRP including retro-inverso forms may be identified using routine techniques known in the art. Prodrugs include compounds wherein an amino acid residue, or a polypeptide chain of two or more (e.g., two, three or four) amino acid residues is covalently joined through an amide or ester bond to a free amino, hydroxy or carboxylic acid group of compounds of the present invention. Additional types of prodrugs are also encompassed. For instance, free carboxyl groups can be derivatized as amides or alkyl esters. Free hydroxy groups may be derivatized using groups including but not limited to hemisuccinates, phosphate esters, dimethylaminoacetates, and phosphoryloxymethyloxycarbonyls, as outlined in Advanced Drug Delivery Reviews 1996, 19, 115. Carbamate prodrugs of hydroxy and amino-groups are also included, as are carbonate prodrugs, sulfonate esters and sulfate esters of hydroxy groups. Derivatization of hydroxy groups as (acyloxy)methyl and (acyloxy)ethyl ethers wherein the acyl group may be an alkyl ester, optionally substituted with groups including but not limited to ether, amine and carboxylic acid functionalities, or where the acyl group is an amino acid ester as described above, are also encompassed. Prodrugs of this type are described in J. Med. Chem. 1996, 39, 10. Free amines can also be derivatized as amides, sulfonamides or phosphonamides. All of these prodrug moieties may incorporate groups including but not limited to ether, amine and carboxylic acid functionalities. Prodrugs of retro-inverso forms of peptides can also be prepared in similar ways, using sidechain derivatization and the like, as described above. Other examples of such prodrug derivatives are described in (a) Design of Prodrugs, edited by H. Bundgaard, (Elsevier, 1985) and Methods in Enzymology, Vol. 42, p. 309-396, edited by K. Widder, et al. (Academic Press, 1985); (b) A Textbook of Drug Design and Development, edited by Krogsgaard-Larsen and H. Bundgaard, Chapter 5; “Design and Application of Prodrugs”, by H. Bundgaard p. 113-191 (1991); (c) H. Bundgaard, Advanced Drug Delivery Reviews, 8:1-38 (1992); (d) H. Bundgaard, et al., J. Pharmaceutical Sciences, 77:285 (1988); and (e) N. Kakeya et al., Chem. Pharm. Bull., 32:692 (1984), each of which is specifically incorporated herein by reference.

The phrase “pharmaceutically acceptable salt(s)”, as used herein, means those salts of compounds of sequence modified CGRPs including corresponding retro-inverso forms that are safe and effective for use in mammals and that possess the desired biological activity. Pharmaceutically acceptable salts include salts of acidic or basic groups present in compounds of the invention. Pharmaceutically acceptable acid addition salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts. Certain compounds of the invention can form pharmaceutically acceptable salts with various amino acids. Suitable base salts include, but are not limited to, aluminum, calcium, lithium, magnesium, potassium, sodium, zinc, and diethanolamine salts. For a review on pharmaceutically acceptable salts see Berge et al., J. Pharm. Sci., 66:1-19 (1977), incorporated herein by reference.

Because CGRP is a naturally occurring vasodilator substance in the human body, sequence modified CGRP does not have the same toxicity and allergy problems as the foreign substances that currently are used for similar purposes. It is also believed that due to the fact that retro-inverso forms of peptides assume solution conformations closely resembling the solution conformations of the normal peptides, those forms also may not induce allergic responses.

It is within ordinary skill to evaluate any sequence modified CGRP disclosed and claimed herein for effectiveness and in various cellular assays using the procedures described herein. Accordingly, the person of ordinary skill can prepare and evaluate any and all of the claimed compounds without undue experimentation. Any sequence modified CGRP found to be effective in vitro can likewise be tested in animal models and in human clinical studies using the skill and experience of the investigator to guide the selection of dosages and treatment regimens.

### Sequence Modified CGRP Preparations

The sequence modified CGRPs including corresponding retro-inverso forms can be synthesized by any suitable method, such as by exclusively solid-phase techniques, by partial solid-phase techniques, by fragment condensation or by classical solution addition. Sequence modified CGRP may also be synthesized by recombinant DNA techniques that may be used for large-scale production. Recombinant DNA techniques are likely not suitable for preparation of retro-inverso forms.

### Synthetic Methods

The sequence modified CGRPs of the invention may be prepared by solid phase chemical peptide synthesis methods. Such methods have been known in the art since the early 1960's. (See Merrifield, 1963; Stewart & Young, Freeman & Co., San Francisco, 1969, and examples in disclosure of U.S. Pat. No. 4,105,603, each of which is herein incorporated by reference in its entirety. In some embodiments, sequence modified CGRP may be obtained using an automatic peptide synthesizer according to well-known methods. One method for synthesizing sequence modified CGRP is the well-known Merrifield method (see, Merrifield, R. B., J. Am. Chem. Soc. 85:2149 (1963) and Merrifield, R. B., Science, 232:341 (1986), which are specifically incorporated herein by reference). See also Wimalawansa, S. J., “Use of synthetic peptides in specific affinity chromatography for purification of specific peptide receptors”, Innovation and Perspectives in Solid Phase Synthesis (Peptides, Polypeptides and Oligonucleotides), (Ed.) R. Epton, Intercept Ltd., Andover, UK (1991) 111-119, incorporated herein by reference. Either t-Boc, F-Moc, or fast-Moc solid-phase peptide chemistry may be used to synthesize the peptide. Similarly, such methods can be used without modification to prepare retro-inverso forms, with the exception that protected forms of D-amino acids, not of L-amino acids as in normal peptides, are used. The sequence is programmed to be the opposite or reverse of the sequence of the corresponding normal peptide. The protected D-amino acids can incorporate the same N-protecting groups, sidechain protecting groups, and carboxylate activation chemistries as in the case of the normal peptide. Conditions that avoid racemization are desirable, as is also the case in the synthesis of normal peptides. When pseudo-peptide (ψ) bonds are used in the sequence, these can be introduced in the same manner as in the case of normal peptides incorporating ψ bonds, as is well known in the art. Deblocking methods are the same as for normal peptides, and are selected based on the particular protecting groups used in the retro-inverso synthesis.

The fragment condensation method of synthesis is exemplified in U.S. Pat. No. 3,972,859 (Aug. 3, 1976). Other available syntheses are exemplified by U.S. Pat. No. 3,842,067 (Oct. 15, 1974) and U.S. Pat. No. 3,862,925 (Jan. 28, 1975), all herein incorporated by reference in their entirety, all of which have been employed in commercially available laboratory peptide design and synthesis kits (Cambridge Research Biochemicals). Commercially available laboratory kits have generally utilized the teachings of Geysen et al. (1984) and provide for synthesizing peptides upon the tips of a multitude of “rods” or “pins” all of which are connected to a single plate. When such a system is utilized, a plate of rods or pins is inverted and inserted into a second plate of corresponding wells or reservoirs, which contain solutions for attaching or anchoring an appropriate amino acid to the pin's or rod's tips. By repeating such a process step, e.g., inverting and inserting the rod's and pin's tips into appropriate solutions, amino acids are built into desired peptides. In addition, a number of available FMOC peptide synthesis systems are available. For example, assembly of a polypeptide or fragment can be carried out on a solid support using an Applied Biosystems, Inc. Model 431A automated peptide synthesizer. Such equipment provides ready access to the peptides of the invention, either by direct synthesis or by synthesis of a series of fragments that can be coupled using other known techniques. Fragment synthesis techniques can also be employed in the preparation of retro-inverso forms, simply substituting fragments composed of the corresponding D-amino acids in the reversed sequence with respect to the normal peptide fragments. Coupling chemistries and deprotection chemistries are the same as in the case of the corresponding normal peptides.

Common to coupling-type chemical syntheses of peptides is the protection of the labile side chain groups of the various amino acid moieties with suitable protecting groups which prevent a chemical reaction from occurring at that site until the group is ultimately removed. Usually also common is the protection of an alpha-amino group on an amino acid or a fragment while that entity reacts at the carboxyl group, followed by the selective removal of the alpha-amino protecting group to allow subsequent reaction to take place at that location. Accordingly, it is common that, as a step in the synthesis, an intermediate compound is produced which includes each of the amino acid residues located in its desired sequence in the peptide chain with various of these residues having side-chain protecting groups.

In selecting a particular side chain protecting group to be used in the synthesis of the peptides, the following rules are followed: (a) the protecting group should be stable to the reagent and under the reaction conditions selected for removing the alpha-amino protecting group at each step of the synthesis, (b) the protecting group should retain its protecting properties and not be split off under coupling conditions and (c) the side chain protecting group must be removable, upon the completion of the synthesis containing the desired amino acid sequence, under reaction conditions that will not alter the peptide chain.

Solid-phase synthesis of both normal peptides and of retro-inverso peptides is commenced from the C-terminal end of the peptide by coupling a protected alpha-amino acid to a suitable resin as generally set forth in U.S. Pat. No. 4,244,946 issued Jan. 21, 1981 to Rivier et al., the disclosure of which is incorporated herein by reference. Such a starting material for sequence modified CGRP can be prepared by attaching alpha-amino-protected Phe to a BHA resin.

Phe protected by BOC is coupled to the BHA resin using methylene chloride or dimethylformamide (DMF) as solvent with a suitable coupling reagent. The selection of an appropriate coupling reagent is within the skill of the art. Particularly suitable as a coupling reagent is N,N′-dicyclohexyl carbodiimide (DCCl). The activating reagents used in the solid phase synthesis of the peptides are well known in the peptide art. Examples of suitable activating reagents are carbodiimides, such as N,N′-diisopropyl carbodiimide and N-ethyl-N′-(3-dimethylaminopropyl)carbodiimide. Other activating reagents and their use in peptide coupling are described by Schroder & Lubke, “The Peptides”, 1 (Academic Press 1965) in Chapter III and by Kapoor, J. Phar. Sci., 59, pp 1-27 (1970), herein incorporated by reference in its entirety.

Following the coupling of BOC-Phe to the resin support, the alpha-amino protecting group is removed, as by using trifluoroacetic acid (TFA) in methylene chloride, TFA alone or with HCl in dioxane. Preferably 50 weight % TFA in methylene chloride is used with 0-5 weight % 1,2 ethanedithiol. The deprotection is carried out at a temperature between about 0° C. and room temperature. Other standard cleaving reagents and conditions for removal of specific alpha-amino protecting groups may be used as described in Schroder & Lubke, supra, pp 72-75 (Academic Press 1965).

After removal of the alpha-amino protecting group, the remaining alpha-amino- and side chain-protected amino acids are coupled step-wise in the desired order to obtain the intermediate compound defined hereinbefore. As an alternative to adding each amino acid separately in the synthesis, some of them may be coupled to one another prior to addition to the solid phase reactor. Each protected amino acid or amino acid sequence is introduced into the solid phase reactor in about a fourfold excess, and the coupling is carried out in a medium of dimethylformamide(DMF):CH₂Cl₂ (1:1) or in DMF or CH₂Cl₂ alone. In instances where the coupling is carried out manually, the success of the coupling reaction at each stage of the synthesis is monitored by the ninhydrin reaction, as described by E. Kaiser et al., Anal. Biochem. 34, 595 (1970), incorporated herein by reference in its entirety. In cases where incomplete coupling occurs, the coupling procedure is repeated before removal of the alpha-amino protecting group prior to the coupling of the next amino acid. The coupling reactions can be performed automatically, as on a Beckman 990 automatic synthesizer, using a program such as that reported in Rivier et al., Biopolymers, 1978, 17, pp. 1927-1938, incorporated herein by reference.

After the desired amino acid sequence has been completed, the intermediate peptide is removed from the resin support by treatment with a reagent, such as liquid hydrogen fluoride, which not only cleaves the peptide from the resin but also cleaves the alpha-amino protecting group X′ and all remaining side chain protecting groups X³, X⁴, X⁵, X⁶, X⁷ and X⁸, except X² when Acm is employed, to obtain the peptide with its Cys residues still protected. The cyclic form of the peptide is obtained by oxidatively removing the protecting acetoamido-methyl groups using an iodine solution, preferably as described by Kamber, et al., Helv. Chem. Acta., 63, 899 (1980), herein incorporated by reference. Alternatively MeOBzl may be used to protect Cys, and oxidation may be carried out using potassium ferricyanide, as described by Rivier et al., Biopolymers, 17 (1978), 1927-38, or by air oxidation, or in accordance with other known procedures.

The following paragraphs set forth a preferred method for synthesizing the sequence modified CGRP of the invention by the solid-phase technique that generally is in accordance with the procedure set forth in U.S. Pat. No. 4,415,558 (Vale, et al.) issued Nov. 15, 1983, the disclosure of which is incorporated herein by reference. Synthesis of retro-inverso peptides can be carried out using these techniques, as well as using techniques described in M. D. Fletcher and M. M. Campbell (1998), Chem. Rev. 98, 763-795, and references cited therein, which are all incorporated by reference herein.

The synthesis of sequence modified CGRP is conducted in a stepwise manner a benzhydrylamine hydrochloride resin, such as available from Bachem, Inc., having a substitution range of about 0.1 to 0.5 mmoles/gm. resin. The synthesis is performed on an automatic Beckman 990B peptide synthesizer. Coupling of BOC-Phe results in the substitution of about 0.35 mmol Phe per gram of resin. All solvents that are used are carefully degassed, preferably by sparging with an inert gas, e.g. helium or nitrogen. The program used is generally that reported in Rivier, J. J. Liquid Chromatogr., 1, 343-367 (1978).

After deprotection and neutralization, the peptide chain is built step-by-step on the resin. Generally, one to two mmol. of BOC-protected amino acid in methylene chloride is used per gram of resin, plus one equivalent of 2 molar DCCl in methylene chloride, for two hours. When BOC-Arg(Tos) is being coupled, a mixture of 50% DMF and methylene chloride is used. Bzl is used as the hydroxyl side-chain protecting group for Ser and Thr. Acm is used to protect the sulfhydryl group of Cys. P-nitrophenyl ester(ONp) is used to activate the carboxyl end of Asn, and for example, BOC-Asn(ONp) is coupled overnight using one equivalent of HOBt in a 50% mixture of DMF and methylene chloride. The amido group of Asn is protected by Xan when DCCl coupling is used instead of the active ester method. 2-Cl—Z is used as the protecting group for the Lys side chain. Tos is used to protect the guanidino group of Arg and the imidazole group of His, and the side chain carboxyl group of Asp is protected by OBzl.

In order to cleave and substantially deprotect the resulting protected peptide-resin, it is treated with 1.5 ml. anisole, 0.5 ml. of methylethylsulfide and 15 ml. hydrogen fluoride per gram of peptide-resin, first at −20° C. for 20 min. and then at 0° C. for one-half hour. After elimination of the hydrogen fluoride under high vacuum, the resin-peptide is washed alternately with dry diethyl ether and chloroform, and the peptides are then extracted with de-gassed 2N aqueous acetic acid and separated from the resin by filtration.

The cleaved peptide is then purified by HPLC and treated with an iodide solution to deprotect the Cys residues and form the disulfide bond between the Cys residues. After cyclization, the peptide is rechromatographed for final purification using semi-preparative HPLC as described in Rivier et al., “Peptides: Structure and Biological Function” (1979) pp. 125-128. The chromatographic fractions are carefully monitored by HPLC, and only the fractions showing substantial purity are pooled.

### Recombinant Methods

Sequence modified CGRP may also be synthesized by recombinant DNA techniques. Recombinant procedures for production of sequence modified CGRP can employ expression systems for small or large scale production of sequence modified CGRP. Expression systems useful for making sequence modified CGRP include, but are not limited to, cells or microorganisms that are transformed with a recombinant nucleic acid construct expression cassette that contains a nucleic acid segment encoding a sequence modified CGRP. Examples of recombinant nucleic acid constructs may include bacteriophage DNA, plasmid DNA, cosmid DNA, or viral expression vectors. Nucleic acid constructs and expression cassettes can be created through use of recombinant methods that are available in the art. [Sambrook and Russell, Molecular Cloning: A Laboratory Manual, 3rd edition (Jan. 15, 2001) Cold Spring Harbor Laboratory Press, ISBN: 0879695765; Ausubel et al., Current Protocols in Molecular Biology, Green Publishing Associates and Wiley Interscience, NY (1989)].

Due to the redundancy of the DNA code there are numerous DNA sequences that would produce a particular amino acid sequence when expressed. Given an amino acid sequence, such as for example Sequence Modified CGRP Compounds (I)-(XII), one can substitute into the DNA sequence alternative codons for the desired amino acids to produce an alternative DNA sequence also coding for the same protein. One may find that particular chimeric cells of a particular expression method favor particular mRNA codons for a particular amino acid. Altering the human DNA sequence to increase the frequency of favored codons may improve the expression efficacy in a chimeric cell, thus improving the efficacy of the expression process. It is impractical to attempt to list all the DNA sequences that may code for the sequence modified CGRP compounds of the invention. However, the invention comprises the novel proteins, their novel amino acid sequences, and all DNA sequences natural or synthetic coding for the novel amino acid sequences.

The alteration of the DNA by codon substitution may be repeated to substitute substantial portions of the original DNA sequence with alternative codons without altering the protein amino acid sequence of the sequence modified CGRP. Alteration of a DNA sequence which produces no change in the protein expressed by the DNA sequence might, for example, be conducted to increase protein expression in a particular host cell by increasing the occurrence of codons that correspond to amino acid tRNAs found in higher concentration in the host cell. Such altered DNA sequences for substitution analogs can be easily produced by those of ordinary skill in the art. Substitution analogs can be obtained by substitution of oligonucleotides at restriction cleavage sites as described above, or by other equivalent methods that change the codons while preserving the amino acid sequence of the expressed protein.

Optimized nucleic acid sequences, e.g., human codon optimized sequences, encoding at least the sequence modified CGRP are employed in the nucleic acid molecules of the invention, as those optimized sequences can increase the stability of the sequence modified CGRP. The optimization of nucleic acid sequences is known to the art, see, for example, WO 02/16944.

In one embodiment, a polynucleotide of the invention is optimized for expression in a particular host. As used herein, optimization includes codon optimization as well as, in eukaryotic cells, introduction of a Kozak sequence, and/or one or more introns. Preferred codons for use in the invention are those which are employed more frequently than at least one other codon for the same amino acid in a particular organism and, more preferably, are also not low-usage codons in that organism and are not low-usage codons in the organism used to clone or screen for the expression of the nucleic acid molecule. Moreover, preferred codons for certain amino acids (i.e., those amino acids that have three or more codons), may include two or more codons that are employed more frequently than the other (non-preferred) codon(s). The presence of codons in the nucleic acid molecule that are employed more frequently in one organism than in another organism results in a nucleic acid molecule which, when introduced into the cells of the organism that employs those codons more frequently, is expressed in those cells at a level that is greater than the expression of the wild-type or parent nucleic acid sequence in those cells.

In one embodiment of the invention, the codons that are different are those employed more frequently in a mammal, while in another embodiment the codons that are different are those employed more frequently in a plant. A particular type of mammal, e.g., human, may have a different set of preferred codons than another type of mammal. Likewise, a particular type of plant may have a different set of preferred codons than another type of plant. In one embodiment of the invention, the majority of the codons which differ are ones that are preferred codons in a desired host cell. Preferred codons for mammals (e.g., humans) and plants are known to the art (e.g., Wada et al., 1990). For example, preferred human codons include, but are not limited to, CGC (Arg), CTG (Leu), TCT (Ser), AGC (Ser), ACC (Thr), CCA (Pro), CCT (Pro), GCC (Ala), GGC (Gly), GTG (Val), ATC (Ile), ATT (Ile), AAG (Lys), AAC (Asn), CAG (Gln), CAC (His), GAG (Glu), GAC (Asp), TAC (Tyr), TGC (Cys) and TTC (Phe) (Wada et al., 1990). Thus, preferred “humanized” synthetic nucleic acid molecules of the invention have a codon composition which differs from a wild type nucleic acid sequence by having an increased number of the preferred human codons, e.g. CGC, CTG, TCT, AGC, ACC, CCA, CCT, GCC, GGC, GTG, ATC, ATT, AAG, AAC, CAG, CAC, GAG, GAC, TAC, TGC, TTC, or any combination thereof. For example, the nucleic acid molecule of the invention may have an increased number of CTG or TTG leucine-encoding codons, GTG or GTC valine-encoding codons, GGC or GGT glycine-encoding codons, ATC or ATT isoleucine-encoding codons, CCA or CCT proline-encoding codons, CGC or CGT arginine-encoding codons, AGC or TCT serine-encoding codons, ACC or ACT threonine-encoding codon, GCC or GCT alanine-encoding codons, or any combination thereof, relative to the wild-type nucleic acid sequence. Similarly, nucleic acid molecules having an increased number of codons that are employed more frequently in plants, have a codon composition which differs from a wild-type nucleic acid sequence by having an increased number of the plant codons including, but not limited to, CGC (Arg), CTT (Leu), TCT (Ser), TCC (Ser), ACC (Thr), CCA (Pro), CCT (Pro), GCT (Ser), GGA (Gly), GTG (Val), ATC (Ile), ATT (Ile), AAG (Lys), AAC (Asn), CAA (Gln), CAC (His), GAG (Glu), GAC (Asp), TAC (Tyr), TGC (Cys), TTC (Phe), or any combination thereof (Murray et al., 1989). Preferred codons may differ for different types of plants (Wada et al., 1990).

DNA clone sequences for sequence modified CGRP are derived from the analog formula: Ala-Cys-X-Thr-Ala-Thr-Cys-Val-Thr-His-Arg-Leu-Ala-X-Leu-Leu-Ser-Arg-Ser-Gly-Gly-X-Val-Lys-X-Asn-Phe-Val-Pro-Thr-Asn-Val-Gly-Ser-Lys-Ala-Phe —NH₂ (SEQ ID NO:51) where X is substituted for one or more amino acids.

Generally, recombinant methods involve preparation of a desired DNA fragment and ligation of that DNA fragment into a preselected position in another DNA vector, such as a plasmid. In a typical example, a desired DNA fragment is first obtained by synthesizing and/or digesting a DNA that contains the desired DNA fragment with one or more restriction enzymes that cut on both sides of the desired DNA fragment. The restriction enzymes may leave a “blunt” end or a “sticky” end. A “blunt” end means that the end of a DNA fragment does not contain a region of single-stranded DNA. A DNA fragment having a “sticky” end means that the end of the DNA fragment has a region of single-stranded DNA. The sticky end may have a 5′ or a 3′ overhang. Numerous restriction enzymes are commercially available and conditions for their use are also well known. (USB, Cleveland, Ohio; New England Biolabs, Beverly, Mass.).

The digested DNA fragments may be extracted according to known methods, such as phenol/chloroform extraction, to produce DNA fragments free from restriction enzymes. The restriction enzymes may also be inactivated with heat or other suitable means. Alternatively, a desired DNA fragment may be isolated away from additional nucleic acid sequences and restriction enzymes through use of electrophoresis, such as agarose gel or polyacrylamide gel electrophoresis. Generally, agarose gel electrophoresis is used to isolate large nucleic acid fragments while polyacrylamide gel electrophoresis is used to isolate small nucleic acid fragments. Such methods are used routinely to isolate DNA fragments. The electrophoresed DNA fragment can then be extracted from the gel following electrophoresis through use of many known methods, such as electroelution, column chromatography, or binding of glass beads. Many kits containing materials and methods for extraction and isolation of DNA fragments are commercially available. (Qiagen, Venlo, Netherlands; Qbiogene, Carlsbad, Calif.).

The DNA segment into which the fragment is going to be inserted is then digested with one or more restriction enzymes. Preferably, the DNA segment is digested with the same restriction enzymes used to produce the desired DNA fragment. This will allow for directional insertion of the DNA fragment into the DNA segment based on the orientation of the complimentary ends. For example, if a DNA fragment is produced that has an EcoRI site on its 5′ end and a BamHI site at the 3′ end, it may be directionally inserted into a DNA segment that has been digested with EcoRI and BamHI based on the complementarity of the ends of the respective DNAs. Alternatively, blunt ended cloning may be used if no convenient restriction sites exist that allow for directional cloning. For example, the restriction enzyme BsaAI leaves DNA ends that do not have a 5′ or 3′ overhang. Blunt ended cloning may be used to insert a DNA fragment into a DNA segment that was also digested with an enzyme that produces a blunt end. Additionally, DNA fragments and segments may be digested with a restriction enzyme that produces an overhang and then treated with an appropriate enzyme to produce a blunt end. Such enzymes include polymerases and exonucleases. Those of skill in the art know how to use such methods alone or in combination to selectively produce DNA fragments and segments that may be selectively combined.

A DNA fragment and a DNA segment can be combined though conducting a ligation reaction. Ligation links two pieces of DNA through formation of a phosphodiester bond between the two pieces of DNA. Generally, ligation of two or more pieces of DNA occurs through the action of the enzyme ligase when the pieces of DNA are incubated with ligase under appropriate conditions. Ligase and methods and conditions for its use are well known in the art and are commercially available.

The ligation reaction or a portion thereof is then used to transform cells to amplify the recombinant DNA formed, such as a plasmid having an insert. Methods for introducing DNA into cells are well known and are disclosed herein.

Those of skill in the art recognize that many techniques for producing recombinant nucleic acids can be used to produce an expression cassette or nucleic acid construct of the invention containing a single copy or multiple copies of the sequence modified CGRP.

### Promoters

The recombinant nucleic acid construct expression cassette of the invention includes a promoter. Any promoter able to direct transcription of the expression cassette may be used. Accordingly, many promoters may be included within the expression cassette of the invention. Some useful promoters include constitutive promoters, inducible promoters, regulated promoters, cell specific promoters, viral promoters, and synthetic promoters. A promoter is a nucleotide sequence which controls expression of an operably linked nucleic acid sequence by providing a recognition site for RNA polymerase, and possibly other factors, required for proper transcription. A promoter includes a minimal promoter, consisting only of all basal elements needed for transcription initiation, such as a TATA-box and/or other sequences that serve to specify the site of transcription initiation. A promoter may be obtained from a variety of different sources. For example, a promoter may be derived entirely from a native gene, be composed of different elements derived from different promoters found in nature, or be composed of nucleic acid sequences that are entirely synthetic. A promoter may be derived from many different types of organisms and tailored for use within a given cell.

Examples of Promoters Useful in Bacteria. For expression of a protein in a bacterium, an expression cassette having a bacterial promoter will be used. A bacterial promoter is any DNA sequence capable of binding bacterial RNA polymerase and initiating the downstream (3′) transcription of a coding sequence into mRNA. A promoter will have a transcription initiation region which is usually placed proximal to the 5′ end of the coding sequence. This transcription initiation region usually includes an RNA polymerase binding site and a transcription initiation site. A second domain called an operator may be present and overlap an adjacent RNA polymerase binding site at which RNA synthesis begins. The operator permits negatively regulated (inducible) transcription, as a gene repressor protein may bind the operator and thereby inhibit transcription of a specific gene. Constitutive expression may occur in the absence of negative regulatory elements, such as the operator. In addition, positive regulation may be achieved by a gene activator protein binding sequence, which, if present is usually proximal (5′) to the RNA polymerase binding sequence. An example of a gene activator protein is the catabolite activator protein (CAP), which helps initiate transcription of the lac operon in E. coli [Raibaud et al., Ann. Rev. Genet., 18:173 (1984)]. Regulated expression may therefore be positive or negative, thereby either enhancing or reducing transcription. A preferred promoter is the YX Chlorella virus promoter. [U.S. Pat. No. 6,316,224].

Sequences encoding metabolic pathway enzymes provide particularly useful promoter sequences. Examples include promoter sequences derived from sugar metabolizing enzymes, such as galactose, lactose (lac) (Chang et al., Nature, 198:1056 (1977)], and maltose. Additional examples include promoter sequences derived from biosynthetic enzymes such as tryptophan (trp) [Goeddel et al., Nuc. Acids Res., 8:4057 (1980); Yelverton et al., Nuc. Acids Res., 9:731 (1981); U.S. Pat. No. 4,738,921; and EPO Publ. Nos. 036 776 and 121 775]. The beta-lactamase (bla) promoter system [Weissmann, “The cloning of interferon and other mistakes”, in: Interferon 3 (ed. I. Gresser), 1981], and bacteriophage lambda PL [Shimatake et al., Nature, 292:128 (1981)] and T5 (U.S. Pat. No. 4,689,406) promoter systems also provide useful promoter sequences.

Synthetic promoters which do not occur in nature also function as bacterial promoters. For example, transcription activation sequences of one bacterial or bacteriophage promoter may be joined with the operon sequences of another bacterial or bacteriophage promoter, creating a synthetic hybrid promoter [U.S. Pat. No. 4,551,433]. For example, the tac promoter is a hybrid tip-lac promoter comprised of both trp promoter and lac operon sequences that is regulated by the lac repressor [Amann et al., Gene, 25:167 (1983); de Boer et al., Proc. Natl. Acad. Sci. USA, 80:21 (1983)]. Furthermore, a bacterial promoter can include naturally occurring promoters of non-bacterial origin that have the ability to bind bacterial RNA polymerase and initiate transcription. A naturally occurring promoter of non-bacterial origin can also be coupled with a compatible RNA polymerase to produce high levels of expression of some genes in prokaryotes. The bacteriophage T7 RNA polymerase/promoter system is an example of a coupled promoter system [Studier et al., J. Mol. Biol., 189:113 (1986); Tabor et al., Proc. Natl. Acad. Sci. USA, 82:1074 (1985)]. In addition, a hybrid promoter can also be comprised of a bacteriophage promoter and an E. coli operator region [EPO Publ. No. 267 851].

Examples of Promoters Useful in Insect Cells. An expression cassette having a baculovirus promoter can be used for expression of a leader protein in an insect cell. A baculovirus promoter is any DNA sequence capable of binding a baculovirus RNA polymerase and initiating transcription of a coding sequence into mRNA. A promoter will have a transcription initiation region which is usually placed proximal to the 5′ end of the coding sequence. This transcription initiation region usually includes an RNA polymerase binding site and a transcription initiation site. A second domain called an enhancer may be present and is usually distal to the structural gene. A baculovirus promoter may be a regulated promoter or a constitutive promoter. Useful promoter sequences may be obtained from structural genes that are transcribed at times late in a viral infection cycle. Examples include sequences derived from the gene encoding the baculoviral polyhedron protein [Friesen et al., “The Regulation of Baculovirus Gene Expression”, in: The Molecular Biology of Baculoviruses (ed. Walter Doerfler), 1986; and EPO Publ. Nos. 127 839 and 155 476] and the gene encoding the baculoviral p10 protein [Vlak et al., J. Gen. Virol., 69:765 (1988)].

Examples of Promoters Useful in Yeast. Promoters that are functional in yeast are known to those of ordinary skill in the art. In addition to an RNA polymerase binding site and a transcription initiation site, a yeast promoter may also have a second region called an upstream activator sequence. The upstream activator sequence permits regulated expression that may be induced. Constitutive expression occurs in the absence of an upstream activator sequence. Regulated expression may be either positive or negative, thereby either enhancing or reducing transcription.

Promoters for use in yeast may be obtained from yeast genes that encode enzymes active in metabolic pathways. Examples of such genes include alcohol dehydrogenase (ADH) (EPO Publ. No. 284 044), enolase, glucokinase, glucose-6-phosphate isomerase, glyceraldehyde-3-phosphatedehydrogenase (GAP or GAPDH), hexokinase, phosphofructokinase, 3-phosphoglyceratemutase, and pyruvate kinase (PyK). [EPO Publ. No. 329 203]. The yeast PHO5 gene, encoding acid phosphatase, also provides useful promoter sequences. [Myanohara et al., Proc. Natl. Acad. Sci. USA, 80:1 (1983)].

Synthetic promoters which do not occur in nature may also be used for expression in yeast. For example, upstream activator sequences from one yeast promoter may be joined with the transcription activation region of another yeast promoter, creating a synthetic hybrid promoter. Examples of such hybrid promoters include the ADH regulatory sequence linked to the GAP transcription activation region [U.S. Pat. Nos. 4,876,197 and 4,880,734]. Other examples of hybrid promoters include promoters which consist of the regulatory sequences of either the ADH2, GAL4, GAL10, or PHO5 genes, combined with the transcriptional activation region of a glycolytic enzyme gene such as GAP or PyK [EPO Publ. No. 164 556]. Furthermore, a yeast promoter can include naturally occurring promoters of non-yeast origin that have the ability to bind yeast RNA polymerase and initiate transcription. Examples of such promoters are known in the art. [Cohen et al., Proc. Natl. Acad. Sci. USA, 77:1078 (1980); Henikoff et al., Nature, 283:835 (1981); Hollenberg et al., Curr. Topics Microbiol. Immunol., 96:119 (1981); Hollenberg et al., “The Expression of Bacterial Antibiotic Resistance Genes in the Yeast Saccharomyces cerevisiae”, in: Plasmids of Medical, Environmental and Commercial Importance (eds. K. N. Timmis and A. Puhler), 1979; Mercerau-Puigalon et al., Gene, 11:163 (1980); Panthier et al., Curr. Genet., 2:109 (1980)].

Examples of Promoters Useful in Mammalian Cells. Many mammalian promoters are known in the art that may be used in conjunction with the expression cassette of the invention. Mammalian promoters often have a transcription initiating region, which is usually placed proximal to the 5′ end of the coding sequence, and a TATA box, usually located 25-30 base pairs (bp) upstream of the transcription initiation site. The TATA box is thought to direct RNA polymerase II to begin RNA synthesis at the correct site. A mammalian promoter may also contain an upstream promoter element, usually located within 100 to 200 bp upstream of the TATA box. An upstream promoter element determines the rate at which transcription is initiated and can act in either orientation [Sambrook et al., “Expression of Cloned Genes in Mammalian Cells”, in: Molecular Cloning: A Laboratory Manual, 2nd ed., 1989].

Mammalian viral genes are often highly expressed and have a broad host range; therefore sequences encoding mammalian viral genes often provide useful promoter sequences. Examples include the SV40 early promoter, mouse mammary tumor virus LTR promoter, adenovirus major late promoter (Ad MLP), and herpes simplex virus promoter. In addition, sequences derived from non-viral genes, such as the murine metallothioneih gene, also provide useful promoter sequences. Expression may be either constitutive or regulated.

A mammalian promoter may also be associated with an enhancer. The presence of an enhancer will usually increase transcription from an associated promoter. An enhancer is a regulatory DNA sequence that can stimulate transcription up to 1000-fold when linked to homologous or heterologous promoters, with synthesis beginning at the normal RNA start site. Enhancers are active when they are placed upstream or downstream from the transcription initiation site, in either normal or flipped orientation, or at a distance of more than 1000 nucleotides from the promoter. [Maniatis et al., Science, 236:1237 (1987); Alberts et al., Molecular Biology of the Cell, 2nd ed., 1989]. Enhancer elements derived from viruses are often times useful, because they usually have a broad host range. Examples include the SV40 early gene enhancer [Dijkema et al., EMBO J., 4:761 (1985)] and the enhancer/promoters derived from the long terminal repeat (LTR) of the Rous Sarcoma Virus [Gorman et al., Proc. Natl. Acad. Sci. USA, 79:6777 (1982b)] and from human cytomegalovirus [Boshart et al., Cell, 41:521 (1985)]. Additionally, some enhancers are regulatable and become active only in the presence of an inducer, such as a hormone or metal ion [Sassone-Corsi and Borelli, Trends Genet., 2:215 (1986); Maniatis et al., Science, 236:1237 (1987)].

It is understood that many promoters and associated regulatory elements may be used within the expression cassette of the invention to transcribe an encoded leader protein. The promoters described above are provided merely as examples and are not to be considered as a complete list of promoters that are included within the scope of the invention.

### Translation Initiation Sequence

The expression cassette of the invention may contain a nucleic acid sequence for increasing the translation efficiency of an mRNA encoding a leader protein of the invention. Such increased translation serves to increase production of the leader protein. The presence of an efficient ribosome binding site is useful for gene expression in prokaryotes. In bacterial mRNA a conserved stretch of six nucleotides, the Shine-Dalgarno sequence, is usually found upstream of the initiating AUG codon. [Shine et al., Nature, 254:34 (1975)]. This sequence is thought to promote ribosome binding to the mRNA by base pairing between the ribosome binding site and the 3′ end of Escherichia coli 16S rRNA. [Steitz et al., “Genetic signals and nucleotide sequences in messenger RNA” in: Biological Regulation and Development: Gene Expression (ed. R. F. Goldberger), 1979)]. Such a ribosome binding site, or operable derivatives thereof, are included within the expression cassettes of the invention.

A translation initiation sequence can be derived from any expressed Escherichia coli gene and can be used within an expression cassette of the invention. Preferably the gene is a highly expressed gene. A translation initiation sequence can be obtained via standard recombinant methods, synthetic techniques, purification techniques, or combinations thereof, which are all well known. [Ausubel et al., Current Protocols in Molecular Biology, Green Publishing Associates and Wiley Interscience, NY. (1989); Beaucage and Caruthers, Tetra. Letts., 22:1859 (1981); VanDevanter et al., Nucleic Acids Res., 12:6159 (1984)]. Alternatively, translational start sequences can be obtained from numerous commercial vendors. (Operon Technologies; Life Technologies Inc, Gaithersburg, Md.). In a preferred embodiment, the T7tag leader sequence is used. The T7tag leader sequence is derived from the highly expressed T7 Gene 10 cistron. Other examples of translation initiation sequences include, but are not limited to, the maltose-binding protein (Mal E gene) start sequence [Guan et al., Gene, 67:21 (1997)] present in the pMalc2 expression vector (New England Biolabs, Beverly, Mass.) and the translation initiation sequence for the following genes: thioredoxin gene (Novagen, Madison, Wis.), Glutathione-S-transferase gene (Pharmacia, Piscataway, N.J.), beta-galactosidase gene, chloramphenicol acetyltransferase gene and E. coli Trp E gene [Ausubel et al., 1989, Current Protocols in Molecular Biology, Chapter 16, Green Publishing Associates and Wiley Interscience, NY].

Eukaryotic mRNA does not contain a Shine-Dalgarno sequence. Instead, the selection of the translational start codon is usually determined by its proximity to the cap at the 5′ end of an mRNA. The nucleotides immediately surrounding the start codon in eucaryotic mRNA influence the efficiency of translation. Accordingly, one skilled in the art can determine what nucleic acid sequences will increase translation of a leader protein encoded by the expression cassette of the invention. Such nucleic acid sequences are within the scope of the invention.

### Vectors

Vectors that may be used include, but are not limited to, those able to be replicated in prokaryotes and eukaryotes. Vectors include, for example, plasmids, phagemids, bacteriophages, viruses, cosmids, and F-factors. Viral vectors include herpes viruses, retroviruses, vaccinia viruses, attenuated vaccinia viruses, canary pox viruses, adenoviruses, adeno-associated viruses and lentiviruses. The invention includes any vector into which the expression cassette of the invention may be inserted and replicated in vitro or in vivo. Specific vectors may be used for specific cell types. Additionally, shuttle vectors may be used for cloning and replication in more than one cell type. Such shuttle vectors are known in the art. The nucleic acid constructs may be carried extrachromosomally within a host cell or may be integrated into a host cell chromosome. Numerous examples of vectors are known in the art and are commercially available. (Sambrook and Russell, Molecular Cloning: A Laboratory Manual, 3rd edition (Jan. 15, 2001) Cold Spring Harbor Laboratory Press, ISBN: 0879695765; New England Biolabs, Beverly, Mass.; Stratagene, La Jolla, Calif.; Promega, Madison, Wis.; ATCC, Rockville, Md.; CLONTECH, Palo Alto, Calif.; Invitrogen, Carlsbad, Calif.; Origene, Rockville, Md.; Sigma, St. Louis, Mo.; Pharmacia, Peapack, N.J.; USB, Cleveland, Ohio). These vectors also provide many promoters and other regulatory elements that those of skill in the art may include within the nucleic acid constructs of the invention through use of known recombinant techniques.

Examples of Vectors Useful in Bacteria. A nucleic acid construct for use in a prokaryote host, such as bacteria, will preferably include a replication system allowing it to be maintained in the host for expression or for cloning and amplification. In addition, a nucleic acid construct may be present in the cell in either high or low copy number. Generally, about 5 to about 200, and usually about 10 to about 150, copies of a high copy number nucleic acid construct will be present within a host cell. A host containing a high copy number plasmid will preferably contain at least about 10, and more preferably at least about 20 plasmids. Generally, about 1 to 10, and usually about 1 to 4, copies of a low copy number nucleic acid construct will be present in a host cell. The copy number of a nucleic acid construct may be controlled by selection of different origins of replication according to methods known in the art. Sambrook and Russell, Molecular Cloning: A Laboratory Manual, 3rd edition (Jan. 15, 2001) Cold Spring Harbor Laboratory Press, ISBN: 0879695765.

A nucleic acid construct containing an expression cassette can be integrated into the genome of a bacterial host cell through use of an integrating vector. Integrating vectors usually contain at least one sequence that is homologous to the bacterial chromosome that allows the vector to integrate. Integrations are thought to result from recombinations between homologous DNA in the vector and the bacterial chromosome. For example, integrating vectors constructed with DNA from various Bacillus strains integrate into the Bacillus chromosome [EPO Publ. No. 127 328]. Integrating vectors may also contain bacteriophage or transposon sequences.

Extrachromosomal and integrating nucleic acid constructs may contain selectable markers to allow for the selection of bacterial strains that have been transformed. Selectable markers can be expressed in the bacterial host and may include genes that render bacteria resistant to drugs such as ampicillin, chloramphenicol, erythromycin, kanamycin (neomycin), and tetracycline [Davies et al., Ann. Rev. Microbiol., 32: 469, (1978)]. Selectable markers may also include biosynthetic genes, such as those in the histidine, tryptophan, and leucine biosynthetic pathways.

Numerous vectors, either extra-chromosomal or integrating vectors, have been developed for transformation into many bacteria. For example, vectors have been developed for the following bacteria: B. subtilis [Palva et al., Proc. Natl. Acad. Sci. USA, 79: 5582, (1982); EPO Publ. Nos. 036 259 and 063 953; PCT Publ. No. WO 84/04541], E. coli [Shimatake et al., Nature, 292: 128, (1981); Amann et al., Gene, 40: 183, (1985); Studier et al., J. Mol. Biol., 189: 113, (1986); EPO Publ. Nos. 036 776, 136 829 and 136 907], Streptococcus cremoris [Powell et al., Appl. Environ. Microbiol., 54: 655, (1988)]; Streptococcus lividans [Powell et al., Appl. Environ. Microbiol., 54: 655, (1988)], and Streptomyces lividans [U.S. Pat. No. 4,745,056]. Numerous vectors are also commercially available (New England Biolabs, Beverly, Mass.; Stratagene, La Jolla, Calif.).

Examples of Vectors Useful in Yeast. Many vectors may be used to construct a nucleic acid construct that contains an expression cassette of the invention and that provides for the expression of a protein in yeast. Such vectors include, but are not limited to, plasmids and yeast artificial chromosomes. Preferably the vector has two replication systems, thus allowing it to be maintained, for example, in yeast for expression and in a prokaryotic host for cloning and amplification. Examples of such yeast-bacteria shuttle vectors include YEp24 [Botstein, et al., Gene, 8:17 (1979)], pCl/1 [Brake et al., Proc. Natl. Acad. Sci. USA, 81:4642 (1984)], and YRp17 [Stinchcomb et al., J. Mol. Biol., 158:157 (1982)]. A vector may be maintained within a host cell in either high or low copy number. For example, a high copy number plasmid will generally have a copy number ranging from about 5 to about 200, and usually about 10 to about 150. A host containing a high copy number plasmid will preferably have at least about 10, and more preferably at least about 20. Either a high or low copy number vector may be selected, depending upon the effect of the vector and the leader protein on the host. [Brake et al., Proc. Natl. Acad. Sci. USA, 81:4642 (1984)].

A nucleic acid construct may also be integrated into the yeast genome with an integrating vector. Integrating vectors usually contain at least one sequence homologous to a yeast chromosome that allows the vector to integrate, and preferably contain two homologous sequences flanking an expression cassette of the invention. Integrations appear to result from recombinations between homologous DNA in the vector and the yeast chromosome. [Orr-Weaver et al., Methods in Enzymol., 101:228 (1983)]. An integrating vector may be directed to a specific locus in yeast by selecting the appropriate homologous sequence for inclusion in the vector. One or more nucleic acid constructs may integrate, which may affect the level of recombinant protein produced. [Rine et al., Proc. Natl. Acad. Sci. USA, 80:6750 (1983)]. The chromosomal sequences included in the vector can occur either as a single segment in the vector, which results in the integration of the entire vector, or two segments homologous to adjacent segments in the chromosome and flanking an expression cassette included in the vector, which can result in the stable integration of only the expression cassette.

Extrachromosomal and integrating nucleic acid constructs may contain selectable markers that allow for selection of yeast strains that have been transformed. Selectable markers may include, but are not limited to, biosynthetic genes that can be expressed in the yeast host, such as ADE2, HIS4, LEU2, TRP1, and ALG7, and the G418 resistance gene, which confer resistance in yeast cells to tunicamycin and G418, respectively. In addition, a selectable marker may also provide yeast with the ability to grow in the presence of toxic compounds, such as metal. For example, the presence of CUP 1 allows yeast to grow in the presence of copper ions. [Butt et al., Microbiol. Rev., 51:351 (1987)].

Many vectors have been developed for transformation into many yeasts.

For example, vectors have been developed for the following yeasts: Candida albicans [Kurtz et al., Mol. Cell. Biol., 6:142 (1986)], Candida maltose [Kunze et al., J. Basic Microbiol., 25:141 (1985)], Hansenula polymorpha [Gleeson et al., J. Gen. Microbiol., 132:3459 (1986); Roggenkamp et al., Mol. Gen. Genet., 202:302 (1986)], Kluyveromyces fragilis [Das et al., J. Bacteriol., 158: 1165 (1984)], Kluyveromyces lactis [De Louvencourt et al., J. Bacteriol., 154:737 (1983); van den Berg et al., Bio/Technology, 8:135 (1990)], Pichia guillerimondii [Kunze et al., J. Basic Microbiol., 25:141 (1985)], Pichia pastoris [Cregg et al., Mol. Cell. Biol., 5: 3376, (1985); U.S. Pat. Nos. 4,837,148 and 4,929,555], Saccharomyces cerevisiae [Hinnen et al., Proc. Natl. Acad. Sci. USA, 75:1929 (1978); Ito et al., J. Bacteriol., 153:163 (1983)], Schizosaccharomyces pombe [Beach and Nurse, Nature, 300:706 (1981)], and Yarrowia lipolytica [Davidow et al., Curr. Genet., 10:39 (1985); Gaillardin et al., Curr. Genet., 10:49 (1985)].

Examples of Vectors Useful in Insect Cells. Baculovirus vectors have been developed for infection into several insect cells and may be used to produce nucleic acid constructs that contain an expression cassette of the invention. For example, recombinant baculoviruses have been developed for Aedes aegypti, Autographa californica, Bombyx mori, Drosophila melanogaster, Spodoptera frugiperda, and Trichoplusia ni [PCT Pub. No. WO 89/046699; Carbonell et al., J. Virol., 56:153 (1985); Wright, Nature, 321: 718 (1986); Smith et al., Mol. Cell. Biol., 3: 2156 (1983); and see generally, Fraser et al., In Vitro Cell. Dev. Biol., 25:225 (1989)]. Such a baculovirus vector may be used to introduce an expression cassette into an insect and provide for the expression of a leader protein within the insect cell.

Methods to form a nucleic acid construct having an expression cassette of the invention inserted into a baculovirus vector are well known in the art. Briefly, an expression cassette of the invention is inserted into a transfer vector, usually a bacterial plasmid which contains a fragment of the baculovirus genome, through use of common recombinant methods. The plasmid may also contain a polyhedrin polyadenylation signal [Miller et al., Ann. Rev. Microbiol., 42:177 (1988)] and a prokaryotic selection marker, such as ampicilling resistance, and an origin of replication for selection and propagation in Escherichia coli. A convenient transfer vector for introducing foreign genes into AcNPV is pAc373. Many other vectors, known to those of skill in the art, have been designed. Such a vector is pVL985 [Luckow and Summers, Virology, 17:31 (1989)].

A wild-type baculoviral genome and the transfer vector having an expression cassette insert are transfected into an insect host cell where the vector and the wild-type viral genome recombine. Methods for introducing an expression cassette into a desired site in a baculovirus virus are known in the art. [Summers and Smith, Texas Agricultural Experiment Station Bulletin No. 1555, 1987. Smith et al., Mol. Cell. Biol., 3:2156 (1983); and Luckow and Summers, Virology, 17:31 (1989)]. For example, the insertion can be into a gene such as the polyhedrin gene, by homologous double crossover recombination; insertion can also be into a restriction enzyme site engineered into the desired baculovirus gene [Miller et al., Bioessays, 4:91 (1989)]. The expression cassette, when cloned in place of the polyhedrin gene in the nucleic acid construct, will be flanked both 5′ and 3′ by polyhedrin-specific sequences. An advantage of inserting an expression cassette into the polyhedrin gene is that inclusion bodies resulting from expression of the wild-type polyhedrin gene may be eliminated. This may decrease contamination of leader proteins produced through expression and formation of inclusion bodies in insect cells by wild-type proteins that would otherwise form inclusion bodies in an insect cell having a functional copy of the polyhedrin gene.

The packaged recombinant virus is expressed and recombinant plaques are identified and purified. Materials and methods for baculovirus and insect cell expression systems are commercially available in kit form. (Invitrogen, San Diego, Calif., USA (“MaxBac” kit)). These techniques are generally known to those skilled in the art and fully described in Summers and Smith, Texas Agricultural Experiment Station Bulletin No. 1555, 1987.

Plasmid-based expression systems have also been developed the may be used to introduce an expression cassette of the invention into an insect cell and produce a leader protein. [McCarroll and King, Curr. Opin. Biotechnol., 8:590 (1997)]. These plasmids offer an alternative to the production of a recombinant virus for the production of leader proteins.

Examples of Vectors Useful in Mammalian Cells. An expression cassette of the invention may be inserted into many mammalian vectors that are known in the art and are commercially available. (CLONTECH, Carlsbad, Calif.; Promega, Madison, Wis.; Invitrogen, Carlsbad, Calif.). Such vectors may contain additional elements such as enhancers and introns having functional splice donor and acceptor sites. Nucleic acid constructs may be maintained extrachromosomally or may integrate in the chromosomal DNA of a host cell. Mammalian vectors include those derived from animal viruses, which require trans-acting factors to replicate. For example, vectors containing the replication systems of papovaviruses, such as SV40 [Gluzman, Cell, 23:175 (1981)] or polyomaviruses, replicate to extremely high copy number in the presence of the appropriate viral T antigen. Additional examples of mammalian vectors include those derived from bovine papillomavirus and Epstein-Barr virus. Additionally, the vector may have two replication systems, thus allowing it to be maintained, for example, in mammalian cells for expression and in a prokaryotic host for cloning and amplification. Examples of such mammalian-bacteria shuttle vectors include pMT2 [Kaufman et al., Mol. Cell. Biol., 9:946 (1989)] and pHEBO [Shimizu et al., Mol. Cell. Biol., 6:1074 (1986)].

### Fusion Partners

The polynucleotide of the invention which encodes a sequence modified CGRP may be employed with other nucleic acid sequences, e.g., a native sequence such as a cDNA or one which has been manipulated in vitro, e.g., to prepare N-terminal, C-terminal, or N- and C-terminal fusion proteins, e.g., a fusion with a protein encoded by a different reporter gene including a selectable marker. Many examples of suitable fusion partners are known to the art and can be employed in the practice of the invention.

The N- or C-terminal fusion partner may be a sequence used for purification, e.g., a glutathione S-transferase (GST) or a polyHis sequence, a sequence intended to alter a property of the sequence modified CGRP, e.g., a protein destabilization sequence, a protein or nucleic acid interaction sequence (e.g., a binding sequence), a subcellular localization sequence, or a sequence which has a property which is distinguishable from one or more properties of the sequence modified CGRP in the fusion protein. In one embodiment, the fusion protein comprises a sequence modified CGRP and a fusion partner which is a reporter protein which reporter protein is useful as an intramolecular control, e.g., a fluorescent protein or luciferase. In another embodiment, the invention includes a vector comprising a nucleic acid sequence encoding a fusion protein comprising a sequence modified CGRP of the invention and a nucleic acid fragment which encodes a reporter protein. Fusion partners include but are not limited to affinity domains or other functional protein sequences, such as those having an enzymatic activity. For example, a functional protein sequence may encode a kinase catalytic domain (Hanks and Hunter, 1995), producing a fusion protein that can enzymatically add phosphate moieties to particular amino acids, or may encode a Src Homology 2 (SH2) domain (Sadowski et al., 1986; Mayer and Baltimore, 1993), producing a fusion protein that specifically binds to phosphorylated tyrosines.

Affinity domains are generally peptide sequences that can interact with a binding partner, e.g., such as one immobilized on a solid support. DNA sequences encoding multiple consecutive single amino acids, such as histidine, when fused to the expressed protein, may be used for one-step purification of the recombinant protein by high affinity binding to a resin column, such as nickel sepharose. Sequences encoding peptides, such as the chitin binding domain (which binds to chitin), glutathione-S-transferase (which binds to glutathione), biotin (which binds to avidin and strepavidin), and the like, can also be used for facilitating purification of the sequence modified CGRP. The affinity domain can be separated from the sequence modified CGRP by methods well known in the art, including the use of inteins (protein self-splicing elements (Chong et al., 1997). Exemplary affinity domains include HisV5, HisX6, C-myc, Flag, SteptTag, hemagluttinin, e.g., HA Tag, GST, thioredoxin, cellulose binding domain, chitin binding domain, S-peptide, T7 peptide, SH2 domain, C-end RNA tag, metal binding domains, e.g., zinc binding domains or calcium binding domains such as those from calcium-binding proteins, e.g., calmodulin, troponin C, calcineurin B, myosin light chain, recoverin, S-modulin, visinin, VILIP, neurocalcin, hippocalcin, frequenin, caltractin, calpain large-subunit, S100 proteins, parvalbumin, calbindin D9K, calbindin D28K, and calretinin, inteins, biotin, streptavidin, MyoD, Id, leucine zipper sequences, and maltose binding protein.

Another class of fusion partners includes a protein encoded by a reporter gene, including, but are not limited to, a neo gene, a beta-gal gene, a gus gene, a cat gene, a gpt gene, a hyg gene, a hisD gene, a ble gene, a mprt gene, a bar gene, a nitrilase gene, a galactopyranoside gene, a xylosidase gene, a thymidine kinase gene, an arabinosidase gene, a mutant acetolactate synthase gene (ALS) or acetoacid synthase gene (AAS), a methotrexate-resistant dhfr gene, a dalapon dehalogenase gene, a mutated anthranilate synthase gene that confers resistance to 5-methyl tryptophan (WO 97/26366), an R-locus gene, a beta-lactamase gene, a xylE gene, an alpha-amylase gene, a tyrosinase gene, an anthozoan luciferase (luc) gene, (e.g., a Renilla reniformis luciferase gene), an aequorin gene, a red fluorescent protein gene, or a green fluorescent protein gene. Included within the terms selectable or screenable marker genes are also genes which encode a “secretable marker” whose secretion can be detected as a means of identifying or selecting for transformed cells. Examples include markers which encode a secretable antigen that can be identified by antibody interaction, or even secretable enzymes which can be detected by their catalytic activity. Secretable proteins fall into a number of classes, including small, diffusible proteins detectable, e.g., by ELISA, and proteins that are inserted or trapped in the cell membrane.

The polypeptide can also encode an “inclusion body leader partner” that is operably linked to the peptide of interest. Such an inclusion body leader partner may be linked to the amino-terminus, the carboxyl-terminus or both termini of a precursor polypeptide. Upon expression of the polypeptide, an attached inclusion body leader partner causes the polypeptide to form inclusion bodies within the bacterial host cell. Inclusion body leader partners can be identified, for example, by linking a test inclusion body leader partner to a polypeptide construct. The resulting inclusion body leader partner-polypeptide construct then would be tested to determine whether it forms an inclusion body within a cell.

The amino acid sequence of an inclusion body leader partner can be altered to produce inclusion bodies that facilitate isolation of inclusion bodies that are formed, thereby allowing an attached polypeptide to be purified more easily. For example, the inclusion body leader partner may be altered to produce inclusion bodies that are more or less soluble under a certain set of conditions. Those of skill in the art realize that solubility is dependent on a number of variables that include, but are not limited to, pH, temperature, salt concentration, protein concentration and the hydrophilicity or hydrophobicity of the amino acids in the protein. Thus, an inclusion body leader partner of the invention may be altered to produce an inclusion body having desired solubility under differing conditions.

An inclusion body leader partner may also be altered to produce inclusion bodies that contain polypeptide constructs having greater or lesser self-association. Self-association refers to the strength of the interaction between two or more polypeptides that form an inclusion body. Such self-association may be determined though use of a variety of known methods used to measure protein-protein interactions. Such methods are known in the art and have been described. Freifelder, Physical Biochemistry: Applications to Biochemistry and Molecular Biology, W.H. Freeman and Co., 2nd edition, New York, N.Y. (1982).

Self-adhesion can be used to produce inclusion bodies that exhibit varying stability to purification. For example, greater self-adhesion may be desirable to stabilize inclusion bodies against dissociation in instances where harsh conditions are used to isolate the inclusion bodies from a cell. Such conditions may be encountered if inclusion bodies are being isolated from cells having thick cell walls. However, where mild conditions are used to isolate the inclusion bodies, less self-adhesion may be desirable as it may allow the polypeptide constructs composing the inclusion body to be more readily solubilized or processed. Accordingly, an inclusion body leader partner of the invention may be altered to provide a desired level of self-adhesion for a given set of conditions.

### Host Cells

Host cells producing the recombinant precursor polypeptides for the methods of the invention include prokaryotic and eukaryotic cells of single and multiple cell organisms. Bacteria, fungi, plant, insect, vertebrate and its subclass mammalian cells and organisms may be employed. Single cell cultures from such sources as well as functional tissue and whole organisms can operate as production hosts according to the invention. Examples include E. coli, tobacco plant culture, maize, soybean, fly larva, mice, rats, hamsters, as well as CHO cell cultures, immortal cell lines and the like.

In a preferred embodiment, bacteria are used as host cells. Examples of bacteria include, but are not limited to, Gram-negative and Gram-positive organisms. Escherichia coli is a preferred organism for expression of preselected polypeptides and amplification of nucleic acid constructs. Many publicly available E. coli strains include K-strains such as MM294 (ATCC 31, 466); X1776 (ATCC 31, 537); KS 772 (ATCC 53, 635); JM109; MC1061; HMS174; and the B-strain BL21. Recombination minus strains may be used for nucleic acid construct amplification to avoid recombination events. Such recombination events may remove concatemers of open reading frames as well as cause inactivation of an expression cassette. Furthermore, bacterial strains that do not express a select protease may also be useful for expression of preselected polypeptides to reduce proteolytic processing of expressed polypeptides. Such strains include, for example, Y1090hsdR, which is deficient in the lon protease.

Eukaryotic cells may also be used to produce a preselected polypeptide and for amplifying a nucleic acid construct. Eukaryotic cells are useful for producing a polypeptide when additional cellular processing is desired. For example, a polypeptide may be expressed in a eukaryotic cell when glycosylation of the polypeptide is desired. Examples of eukaryotic cell lines that may be used include, but are not limited to: AS52, H187, mouse L cells, NIH-3T3, HeLa, Jurkat, CHO-K1, COS-7, BHK-21, A-431, HEK293, L6, CV-1, HepG2, HC11, MDCK, silkworm cells, mosquito cells, and yeast.

The glycosylation with a mammalian cell such as a CHO cell is known to differ from that of an insect expression system such as the baculovirus expression vector system. The difference is that glycosylation of a protein molecule derived from the baculovirus vector inserted into an insect expression system leads to an asparagine attached di-N-acetylglycosamine to which a terminal trimannose is attached. This is termed the paucimannose structure and it facilitates interaction with mannose receptors on antigen-presenting cells. Hence, there may be an advantage in some situations to utilize a baculovirus expression vector system. In other embodiments, a mammalian expression system may be used, where additional N-linked glycans may be attached to the three mannoses of the terminal trimarmose (paucimannose) structure generated in the insect expression system. These N-linked glycans include N-acetylglycosamine, galactose, and N-acetylneuraminic acid (also known as sialic acid). Therefore, a variety of host cells can be used to generate sequence modified CGRP polypeptides with somewhat different glycosylation patterns. The invention is directed to compositions and methods of using sequence modified CGRP with any type of glycosylation, or no glycosylation.

### Transformation

Methods for introducing exogenous DNA into bacteria are available in the art, and usually include either the transformation of bacteria treated with CaCl₂ or other agents, such as divalent cations and DMSO. DNA can also be introduced into bacterial cells by electroporation, use of a bacteriophage, or ballistic transformation. Transformation procedures usually vary with the bacterial species to be transformed [see, e.g., Masson et al., FEMS Microbiol. Lett., 60: 273 (1989); Palva et al., Proc. Natl. Acad. Sci. USA, 79: 5582 (1982); EPO Publ. Nos. 036 259 and 063 953; PCT Publ. No. WO 84/04541 [Bacillus], Miller et al., Proc. Natl. Acad. Sci. USA, 8: 856 (1988); Wang et al., J. Bacteriol., 172: 949 (1990) [Campylobacter], Cohen et al., Proc. Natl. Acad. Sci. USA, 69: 2110 (1973); Dower et al., Nuc. Acids Res., 16: 6127 (1988); Kushner, “An improved method for transformation of Escherichia coli with ColE1-derived plasmids”, in: Genetic Engineering: Proceedings of the International Symposium on Genetic Engineering (eds. H. W. Boyer and S, Nicosia), (1978); Mandel et al., J. Mol. Biol., 53: 159 (1970); Taketo, Biochim. Biophys. Acta, 949: 318 (1988) [Escherichia], Chassy et al., FEMS Microbiol. Lett., 44: 173 (1987) [Lactobacillus], Fiedler et al., Anal. Biochem, 170: 38 (1988) [Pseudomonas], Augustin et al., FEMS Microbiol. Lett., 66: 203 (1990) [Staphylococcus], Barany et al., J. Bacteriol., 144: 698 (1980); Harlander, “Transformation of Streptococcus lactis by electroporation”, in: Streptococcal Genetics (ed. J. Ferretti and R. Curtiss III), (1987); Perry et al., Infec. Immun., 32: 1295 (1981); Powell et al., Appl. Environ. Microbiol. 54: 655 (1988); Somkuti et al., Proc. 4th Eur. Cong. Biotechnology, 1: 412 (1987) [Streptococcus]].

Methods for introducing exogenous DNA into yeast hosts are well-known in the art, and usually include either the transformation of spheroplasts or of intact yeast cells treated with alkali cations. Transformation procedures usually vary with the yeast species to be transformed [see, e.g., Kurtz et al., Mol. Cell. Biol., 6:142 (1986); Kunze et al., J. Basic Microbiol., 25:141 (1985) [Candida], Gleeson et al., J. Gen. Microbiol., 132:3459 (1986); Roggenkamp et al., Mol. Gen. Genet., 202:302 (1986) [Hansenula], Das et al., J. Bacteriol., 158:1165 (1984); De Louvencourt et al., J. Bacteriol., 754:737 (1983); Van den Berg et al., Bio/Technology, 8:135 (1990) [Kluyveromyces], Cregg et al., Mol. Cell. Biol., 5:3376 (1985); Kunze et al., J. Basic Microbiol., 25:141 (1985); U.S. Pat. Nos. 4,837,148 and 4,929,555 [Pichia], Hinnen et al., Proc. Natl. Acad. Sci. USA, 75:1929 (1978); Ito et al., J. Bacteriol., 153:163 (1983) [Saccharomyces], Beach and Nurse, Nature, 300:706 (1981) [Schizosaccharomyces], and Davidow et al., Curr. Genet., 10:39 (1985); Gaillardin et al., Curr. Genet., 10:49 (1985) [Yarrowia]].

Methods for introduction of polynucleotides into mammalian cells are known in the art and include lipid-mediated transfection, dextran-mediated transfection, calcium phosphate precipitation, polybrene-mediated transfection, protoplast leader, electroporation, encapsulation of -the polynucleotide(s) in liposomes, biollistics, and direct microinjection of the DNA into nuclei. The choice of method depends on the cell being transformed as certain transformation methods are more efficient with one type of cell than another. [Feigner et al., Proc. Natl. Acad. Sci., 84:7413 (1987); Feigner et al., J. Biol. Chem., 269:2550 (1994); Graham and van der Eb, Virology, 52:456 (1973); Vaheri and Pagano, Virology, 27:434 (1965); Neuman et al., EMBO J., 1:841 (1982); Zimmerman, Biochem. Biophys. Acta., 694:227 (1982); Sanford et al., Methods Enzymol., 217:483 (1993); Kawai and Nishizawa, Mol. Cell. Biol., 4:1172 (1984); Chaney et al., Somat. Cell Mol. Genet., 12:237 (1986); Aubin et al., Methods Mol. Biol., 62:319 (1997)]. In addition, many commercial kits and reagents for transfection of eukaryotic are available.

Following transformation or transfection of a nucleic acid into a cell, the cell may be selected for through use of a selectable marker. A selectable marker is generally encoded on the nucleic acid being introduced into the recipient cell. However, co-transfection of selectable marker can also be used during introduction of nucleic acid into a host cell. Selectable markers that can be expressed in the recipient host cell may include, but are not limited to, genes which render the recipient host cell resistant to drugs such as actinomycin C1, actinomycin D, amphotericin, ampicillin, bleomycin, carbenicillin, chloramphenicol, geneticin, gentamycin, hygromycin B, kanamycin monosulfate, methotrexate, mitomycin C, neomycin B sulfate, novobiocin sodium salt, penicillin G sodium salt, puromycin dihydrochloride, rifampicin, streptomycin sulfate, tetracycline hydrochloride, and erythromycin. [Davies et al., Ann. Rev. Microbiol., 32: 469, (1978)]. Selectable markers may also include biosynthetic genes, such as those in the histidine, tryptophan, and leucine biosynthetic pathways. Upon transfection or transformation of a host cell, the cell is placed into contact with an appropriate selection marker.

For example, if a bacterium is transformed with a nucleic acid construct that encodes resistance to ampicillin, the transformed bacterium may be placed on an agar plate containing ampicillin. Thereafter, cells into which the nucleic acid construct was not introduced would be prohibited from growing to produce a colony while colonies would be formed by those bacteria that were successfully transformed.

### Peptide Amidation

When produced from a single or multicopy polypeptide under normal conditions, the sequence modified CGRP has a C terminal carboxyl group. However, an amidated C-terminus is preferred for use in mammals. Methods of polypeptide amidation will be well known to those of skill in the art. In some embodiments, C-terminally amidated sequence modified CGRP is prepared by solid phase synthesis using an amide derivative of the c-terminal amino acid. In some embodiments, C-terminally amidated sequence modified CGRP is be prepared via condensation reactions of amines. In some embodiments, the C-terminal amino acid of sequence modified CGRP is followed by a glycine, which provides the amide group. In a first reaction step the glycine is oxidized to form alpha-hydroxy-glycine. The oxidized glycine cleaves into the C-terminally amidated peptide and an N-glyoxylated peptide. In some embodiments, clostripain is employed to amidate the C-terminal residue to make an amidated recombinant peptide when the C-terminal amino acid of sequence modified CGRP is followed by an arginine. According to that process, ammonia is included in the clostripain cleavage medium. Transpeptidation will occur at the C-terminal side of the arginine. The ammonia is added to the inchoate C-terminus by the clostripain to form the C-terminus amide. Addition of an amino acid to the C-terminus can also be accomplished by substitution of the amino acid for ammonia in such a clostripain cleavage.

### Sequence Modified CGRP Conjugates

A further aspect of this invention includes conjugates comprising a sequence modified CGRP including corresponding retro-inverso forms coupled to biocompatible polymers, and to methods of treatment by administering a sequence modified CGRP conjugate to a patient. It is known that many potentially therapeutic proteins have been found to have a short half-life in the blood serum. For the most part, proteins are cleared from the serum through the kidneys. Small molecules that normally would be excreted through the kidneys are maintained in the blood stream if their size is increased by attaching a biocompatible polymer such as a PEG derivative. Proteins and other substances that create an immune response when injected can be hidden to some degree from the immune system by coupling of a polymer to the protein. Accordingly, another embodiment of this invention comprises a method of treatment by administering a conjugate comprising sequence modified CGRP including corresponding retro-inverso forms coupled to a biocompatible polymer. Preferably the biocompatible polymer is non-immunigenic. As used herein, the term “conjugate” refers to a sequence modified CGRP molecule including corresponding retro-inverso forms covalently or noncovalently coupled to one or more biocompatible polymers. These conjugates are substantially non-immunogenic and retain at least 75%, preferably 85%, and more preferably 95% or more of the activity of unmodified sequence modified CGRP.

Examples of polymers that can be coupled to sequence modified CGRP including corresponding retro-inverso forms include, but not limited to, biological polymers (e.g., polysaccharides, polyamides, pharmacologically inert nucleotide components, etc.), and derivatives of biological polymers, or non-biological polymers. Specific examples include poly(alkylene glycols) such as poly(ethylene glycol) MPEG), poly-lactic acid (PLA), poly-glycolic acid, poly(ε-caprolactone), poly(β-hydroxybutyrate), poly(β-hydroxyvalerate), polydioxanone, poly(malic acid), poly(tartronic acid), poly(ortho esters), polyanhydrides, polycyanoacrylates, poly(phosphoesters), polyphosphazenes, hyaluronidate, polysulfones, polyacrylamides, polymethacrylate, chimeric recombinant elastin-silk protein (Protein Polymers, Inc.) and collagen (Matrix Pharmaceuticals, Inc.). In a preferred embodiment sequence modified CGRP is conjugated to PEG or a polysaccharide.

As used herein the term “PEG” includes to straight or branched polyethylene glycol oligomer and monomers (PEG subunits) and also includes polyethylene glycol oligomers that have been modified to include groups that do not eliminate the amphiphilic properties of such oligomer, e.g., without limitation, alkyl, lower alkyl, aryl, amino-alkyl and amino-aryl. The term “PEG subunit” refers to a single polyethylene glycol unit, i.e., (—O—CH₂CH₂—).

Sequence modified CGRP including corresponding retro-inverso forms can be conjugated to any of the above-described polymers using conventional methods known to those skilled in the art, wherein the conjugation is performed under conditions which do not substantially reduce the pharmacological activity of sequence modified CGRP or its retro-inverso form. For example, sequence modified CGRP can be covalently coupled to the polymer directly through reaction of a reactive group on the sequence modified CGRP with a reactive group of the polymer. The term “reactive group” refers to a chemical moiety, which is attached to sequence modified CGRP or the polymer, or bonds in the polymer, which participate in the chemical reaction between the components involved, i.e., sequence modified CGRP and the polymer. Examples of reactive groups include without limitation hydroxyl, carboxyl, amine, amide, carbon-carbon double and triple bonds, epoxy groups, halogen or other leaving groups and the like. Alternatively, sequence modified CGRP can be coupled to the polymer through a linking group. The term “linking group” is not limited to molecules per se, and refers to compounds, molecules and molecular fragments that can react with the polymer, monomers and sequence modified CGRP to attach sequence modified CGRP to the polymer. As such, the linking groups include compounds and the like with more than one reactive group, preferably two or three reactive groups.

Reactive sites that form the loci for attachment of polymers to sequence modified CGRP including corresponding retro-inverso forms are dictated by the protein's structure. Many polymers react with free primary amino groups or thiol groups of the polypeptide. Covalent attachment of the polymers to sequence modified CGRP may be accomplished by known chemical synthesis techniques. In one embodiment of the invention, sequence modified CGRP including corresponding retro-inverso forms may be conjugated via a biologically stable, nontoxic, covalent linkage to one or more strands of PEG. Such linkages may include urethane (carbamate) linkages, secondary amine linkages, and amide linkages. Various activated PEGs suitable for such conjugation are available commercially from Shearwater Polymers (Huntsville, Ala.).

In order to use sequence modified CGRPs including corresponding retro-inverso forms for the therapeutic treatment (including prophylactic treatment) of mammals including humans according to the methods of this invention, it is normally formulated in accordance with standard pharmaceutical practice as a pharmaceutical composition. According to this aspect of the invention there is provided a pharmaceutical composition comprising sequence modified CGRP in association with a pharmaceutical diluent or carrier, wherein the sequence modified CGRP is present in an amount for effective treating or preventing HF and/or for improving renal function. Such pharmaceutical compositions include CGRP conjugates in combination with pharmaceutical excipients, diluents, or carriers.

Sequence modified CGRP including corresponding retro-inverso forms can be administered to a patient by any available and effective delivery system including, but not limited to administering sequence modified CGRP to the patient by a method selected from parenteral, transdermal, intranasal, sublingual, transmucosal, intra-arterial, oral, intracoronary, intravenous, transmucosal, topical rectal, vaginal, or intradermal modes of administration in dosage unit formulations containing conventional nontoxic pharmaceutical carriers, adjuvants, and vehicles as desired, such as a depot or a controlled release formulation.

For example, sequence modified CGRP or a pharmaceutical formulation thereof may be formulated for parenteral administration, e.g., for intravenous, subcutaneous, or intramuscular injection. A sterile injectable preparation may be a sterile injectable solution or suspension in a nontoxic parenterally-acceptable diluent or solvent, for example a solution in 1,3-butanediol. For an injectable formulation, a dose of sequence modified CGRP may be combined with a sterile aqueous solution which is preferably isotonic with the blood of the patient. Such a formulation may be prepared by dissolving a solid active ingredient in water containing physiologically-compatible substances such as sodium chloride, glycine, and the like, and having a buffered pH compatible with physiological conditions so as to produce an aqueous solution, and then rendering the solution sterile by, methods known in the art. The formulations may be present in unit or multi-dose containers, such as sealed ampoules or vials. The formulation may be delivered by any mode of injection, including, without limitation, epifascial, intracutaneous, intramuscular, intravascular, intravenous, parenchymatous, subcutaneous, oral or nasal preparations (see, for example, U.S. Pat. No. 5,958,877, which is specifically incorporated herein by reference).

### Controlled Release Sequence Modified CGRP

Another aspect of this invention provides methods of administering sequence modified CGRP including corresponding retro-inverso forms via controlled release formulations. Thus, this invention provides methods of treating heart failure, stroke, migraine, improving renal function, preventing or delaying the advancement of heart failure into advanced stages, treating angina, controlling pulmonary hypertension, counteracting ischemia due to a myocardial infarction, preventing vasospasms during angioplasty, preventing reocclusion of blood vessels during and/or after angioplasty, stent insertion, or the implantation of a vascular grafts, and improving male impotence and female sexual arousal using controlled release formulations containing sequence modified CGRP.

In one embodiment, the controlled release composition comprises a biodegradable polymer matrix containing sequence modified CGRP wherein sequence modified CGRP is released from the polymer matrix (such as a polymer gel matrix) in situ by diffusion or dissolution from within the polymer matrix and/or by the degradation of the polymeric matrix. The controlled release formulation can also be in film form. In another embodiment, the controlled release formulation comprises solid microparticles formed from the combination of biodegradable, synthetic polymers such as poly(lactic acid) (PLA), poly(glycolic acid) (PGA), and copolymers thereof with sequence modified CGRP loadings that yield a sustained release over a period of time when administered orally, transmucosally, topically or by injection. In further embodiments, the controlled release formulations comprise sequence modified CGRP encapsulated in a liposome or sequence modified CGRP conjugated to a polymer.

As used herein, the term “controlled” or “sustained” release of sequence modified CGRP including corresponding retro-inverso forms includes continuous or discontinuous, linear or non-linear release of sequence modified CGRP. There are many advantages for a controlled release formulation of sequence modified CGRP. Among these are the convenience of a single dosage (e.g., injection) for the patient, avoidance of peaks and valleys in systemic sequence modified CGRP concentration which can be associated with repeated injections, the potential to reduce the overall dosage of sequence modified CGRP, delayed progression of the disease, and the potential to enhance the pharmacological effects of sequence modified CGRP. A lower, sustained dose can also prevent adverse affects that: are occasionally observed with infusion therapy. In addition to significantly reducing the cost of care, controlled release drug therapy can free the patient from repeated treatment or hospitalization thus offering the patient greater flexibility and improving patient compliance. A controlled release formulation of sequence modified CGRP also provides an opportunity to use sequence modified CGRP in a manner not previously exploited or considered, such as a maintenance therapeutic for patients that have suffered an MI or in patients at high risk of suffering an MI, such as Stage B, C and D heart failure patients.

### Controlled Release Implants

One embodiment of a controlled release composition of this invention suitable for use in preventing, or treating the diseases described herein comprises a flowable composition that forms a biodegradable implant comprising sequence modified CGRP including corresponding retro-inverso forms in situ. This invention further comprises a kit that includes the flowable composition. The flowable composition comprises a biodegradable, biocompatible thermoplastic polymer or copolymer in combination with a suitable polar solvent and sequence modified CGRP. The thermoplastic polymers or copolymers are substantially insoluble in water and body fluid and are biodegradable and/or bioerodible within the body of an animal. The flowable composition is administered for example as a liquid or gel to a tissue or bodily fluid wherein the implant (i.e., a polymer matrix) is formed in situ, and sequence modified CGRP is subsequently released from the matrix by diffusion or dissolution from within the polymer matrix and/or by the degradation of the polymeric matrix. The composition is biocompatible and the polymer matrix does not cause substantial tissue irritation or necrosis at the implant site. Examples of biocompatible, biodegradable controlled release polymer formulations suitable for purposes of this invention are provided in U.S. Pat. Nos. RE 37,950 E, 6,143,314 and 6,582,080 B2, which are specifically incorporated herein by reference.

More specifically, a flowable thermoplastic polymeric composition of this invention comprises a thermoplastic polymer or copolymer dissolved in a pharmaceutically-acceptable organic solvent that is miscible to dispersible in an aqueous medium to provide a polymeric solution, and sequence modified CGRP or a sequence modified CGRP conjugate either dissolved to form a homogeneous solution or dispersed to form a suspension or a dispersion of sequence modified CGRP within the polymeric solution. When the polymer solution is placed in an aqueous environment, such as a bodily tissue or fluid, which typically surround tissues or organs in an organism, the organic solvent dissipates or disperses into the aqueous or body fluid. Concurrently, the polymer precipitates or coagulates to form a solid matrix or implant and sequence modified CGRP becomes trapped or encapsulated within the polymeric matrix as the implant solidifies. Once the solid implant is formed, sequence modified is released from the solid matrix by diffusion or dissolution from within the polymeric matrix and/or by the degradation of the polymeric matrix. Preferably, the flowable composition is a liquid, gel, paste or putty suitable for injection in a patient.

A thermoplastic composition is provided in which a biodegradable polymer and sequence modified CGRP including corresponding retro-inverso forms are dissolved in a biocompatible solvent to form a flowable composition, which can then be administered, for example, via a syringe and puncture needle or a catheter. Any suitable biodegradable, bioabsorbable, and/or bioerodible thermoplastic polymer can be employed, provided the biodegradable thermoplastic polymer is at least substantially insoluble in aqueous medium or body fluid. Suitable biodegradable thermoplastic polymers are disclosed, e.g., in U.S. Pat. Nos. 5,324,519; 4,938,763; 5,702,716; 5,744,153; and 5,990,194, each of which is specifically incorporated herein by reference. The thermoplastic polymers can be made from a variety of monomers, which form linear or branched polymer chains or monomeric units joined together by linking groups such as esters, amides urethanes, etc. According to one embodiment, some fraction of one of these starting monomers will be at least trifunctional, and provides at lest some branching of the resulting polymer chain. Examples of suitable biodegradable polymers include, but are not limited to, polylactides, polyglycolides, polycaprolactones, polyanhydrides, polyamides, polyorthoesters, polyurethanes, polyesteramides, polydioxanones, polyacetals, polyketals, polycarbonates, polyorthocarbonates, polyphosphazenes, polyhydroxybutyrates, polyhydroxyvalerates, polyalkylene oxalates, polyacrylates, polyalkylene succinates, poly(malic acid), poly(amino acids) and copolymers, terpolymers, cellulose diacetate and ethylene vinyl alcohol copolymers, and combinations thereof.

The type, molecular weight, and amount of biodegradable thermoplastic polymer present in the composition will typically depend upon the desired properties of the controlled release implant. For example, the type, molecular weight, and amount of biodegradable thermoplastic polymer can influence the length of time in which sequence modified CGRP is released from the controlled release implant. Specifically, in one embodiment of the present invention, the composition can be used to formulate a one month delivery system of sequence modified CGRP. In such an embodiment, the biodegradable thermoplastic polymer can preferably be 50/50 poly (DL-lactide-co-glycolide), can be present in about 30 wt. % to about 40 wt. % of the composition, and can have an average molecular weight of about 12,000 to about 45,000. Alternatively, in another embodiment the composition can be used to formulate a three month delivery system of sequence modified CGRP. In such an embodiment, the biodegradable thermoplastic polyester can preferably be 75/25 poly (D,L-lactide-co-glycolide), can be present in about 40 wt. % to about 50 wt. % of the composition, and can have an average molecular weight of about 15,000 to about 24,000.

The molecular weight of the polymer used in the present invention can affect the rate of sequence modified CGRP release in situ from the implant. As the molecular weight of the polymer increases, the rate of sequence modified CGRP release from the system decreases. This phenomenon can be advantageously used in the formulation of systems for the controlled release of sequence modified CGRP. For relatively quick release of sequence modified CGRP, low molecular weight polymers can be chosen to provide the desired release rate. For release of a sequence modified CGRP over a relatively long period of time, a higher polymer molecular weight can be chosen. Accordingly, a polymer system can be produced with an optimum polymer molecular weight range for the release of sequence modified CGRP over a selected length of time. The molecular weight of a polymer can be varied by any of a variety of methods known to persons skilled in the art.

The particular biocompatible polymer employed is not critical and is selected relative to the viscosity of the resulting polymer solution, the solubility of the biocompatible polymer in the biocompatible solvent, the desired release rate, and the like. Such factors are well known to persons skilled in the art. The biodegradable thermoplastic polyester is preferably present in about 30 wt. % to about 50 wt. % of the flowable composition. Preferably, the biodegradable thermoplastic polyester has an average molecular weight of about 12,000 to about 45,000 and more preferably from about 15,000 to about 30,000.

The concentration of the polymer dissolved in the various solvents will differ depending upon the type of polymer and its molecular weight, and these factors can be varied to obtain optimum injection efficiency. sequence modified CGRP is added to the polymer solution where it is either dissolved to form a homogenous solution or dispersed to form a suspension or a dispersion of drug within the polymeric solution.

Suitable organic solvents for preparing the thermoplastic polymer composition are those that are biocompatible, pharmaceutical, and able to diffuse into in aqueous or body fluids so that the flowable composition coagulates or solidifies. The organic solvent is capable of diffusing, dispersing, or leaching from the composition in situ into aqueous tissue or body fluid of the implant site. Examples of suitable solvents include substituted heterocyclic compounds such as N-methyl-2-pyrrolidone and 2 pyrrolidone; esters of carbonic acid and alkyl alcohols such as propylene carbonate, ethylene carbonate and dimethyl carbonate; alkyl esters of mono-, di-, and tricarboxylic acids such as 2-ethyoxyethyl acetate, ethyl acetate, methyl acetate, ethyl lactate, ethyl butyrate, diethyl malonate, diethyl glutonate, tributyl citrate, diethyl succinate, tributyrin, isopropyl myristate, dimethyl adipate, dimethyl succinate, dimethyl oxalate, dimethyl citrate, triethyl citrate, acetyl tributyl citrate, glyceryl triacetate; alkyl ketones such as acetone and methyl ethyl ketone; alcohols such as solketal, glycerol formal, and glycofurol; dialkylamides such as dimethylformamide, dimethylacetamide; dimethylsulfoxide (DMSO) and dimethylsulfone; tetrahydrofuran; lactones such as .epsilon.-caprolactone and butyrolactone; cyclic alkyl amides such as caprolactam; aromatic amides such as N,N-dimethyl-m-toluamide and 1-dodecylazacycloheptan-2-one; and mixtures and combinations thereof. Preferred solvents include polar aprotic solvents such as N-methyl-2-pyrrolidone, 2-pyrrolidone, N-dimethyl formamide, dimethylsulfoxide, caprolactam, triacetin, ethyl lactate, propylene carbonate, solketal, glycerol formal, glycofurol, or any combination thereof.

The solvent can be present in any suitable amount, provided the solvent is miscible to dispersible in aqueous medium or body fluid. The type and amount of biocompatible solvent present in the composition will typically depend upon the desired properties of the controlled release implant. For example, the type and amount of biocompatible solvent can influence the length of time in which the sequence modified CGRP is released from the controlled release polymer matrix. Preferably, the solvent is present in about 45-70 wt. % of the polymeric composition. Specifically, in one embodiment of the present invention, the composition can be used to formulate a one month delivery system of sequence modified CGRP. In such an embodiment, the biocompatible solvent can preferably be N-methyl-2-pyrrolidone and can preferably present in about 60 wt. % to about 70 wt. % of the composition. Alternatively, in another embodiment of the present invention, the composition can be used to formulate a three month delivery system of sequence modified CGRP. In such an embodiment, the biocompatible solvent can preferably be N-methyl-2-pyrrolidone and can preferably present in about 50 wt. % to about 60 wt. % of the composition.

The solubility of the biodegradable thermoplastic polymers in the various solvents will differ depending upon their crystallinity, their hydrophilicity, hydrogen-bonding, bonding, and molecular weight. Thus, not all of the biodegradable thermoplastic polymers will be soluble in the same solvent, and each biodegradable thermoplastic polymer or copolymer will have its appropriate solvent.

A method for forming a flowable polymeric composition includes mixing, in any order, a biodegradable thermoplastic polyester, a biocompatible solvent, and sequence modified CGRP. These ingredients, their properties, and preferred amounts are as disclosed above. The mixing is performed for a sufficient period of time effective to form the flowable composition for use as a controlled release implant. Preferably, the biocompatible thermoplastic polyester and the biocompatible solvent are mixed together to form a mixture and the mixture is then combined with sequence modified CGRP to form the flowable composition. If necessary, gentle heating and stirring can be used to effect dissolution of the biocompatible polymer into the biocompatible solvent.

The amount of sequence modified CGRP incorporated into the polymeric composition depends upon several factors, including but not limited to the desired release profile, the concentration of sequence modified CGRP required for a biological effect, and the length of time that sequence modified CGRP has to be released for effective treatment. There is no critical upper limit on the amount of sequence modified CGRP incorporated into the polymer solution except for that of an acceptable solution or dispersion viscosity for injection through a syringe needle. The lower limit of sequence modified CGRP incorporated into the delivery system is dependent simply upon the activity of the sequence modified CGRP and the length of time needed for treatment.

The release of sequence modified CGRP from the solid polymer matrices (implants) will follow the same general rules for release of a drug from a monolithic polymeric device. The release of sequence modified CGRP can be affected by the size of the implant (i.e., the amount of polymer composition administered to the patient), the loading of sequence modified CGRP within the implant, the permeability factors involving sequence modified CGRP and the particular polymer, and the degradation of the polymer. Depending upon the amount of sequence modified CGRP selected for delivery, the above parameters can be adjusted by one skilled in the art of drug delivery to give the desired rate and duration of release. Thus, the flowable composition can be designed to produce an implant that will release sequence modified CGRP over a targeted period from days to months.

The amount of flowable composition administered will typically depend upon the desired properties of the controlled release implant. For example, the amount of flowable composition can influence the length of time in which sequence modified CGRP is released from the controlled release implant.

It is desirable with any of the controlled release systems or formulations described herein that sequence modified CGRP is delivered to the patient at a rate and in an amount that will achieve blood plasma levels necessary to provide symptomatic relief, e.g., by attenuating one or more symptoms. The following are examples of minimum and maximum IV infusion rates, cumulative daily dose and plasma levels required to bring about the fall range of hemodynamic benefits that sequence modified CGRP induces without any serious side effects in hemodynamically compromised patients. Minimal and transient facial flushing may be observed, but dosages are very well tolerated in IV infusions.

1. Minimum infusion rate and daily dose delivered to cause attenuation of one or more symptoms of HF for a patient weighing 70 kg: 0.0008 μg/kg/min×70 kg×1440 minutes 80.64 μg/day.

2. Maximum infusion rate and daily dose delivered to cause attenuation of one or more symptoms of HF for a patient weighing 70 kg: 0.016 μg/kg/min×70 kg×1440 minutes 1.6 mg/day.

It is well within the skill of persons skilled in the art to determine the amount of sequence modified CGRP to be loaded into a particular drug delivery system to provide the desired steady state plasma levels of sequence modified CGRP as described herein to provide relief of one or more symptoms of HF or to improve one or more hemodynamic properties according to the methods of this invention.

The following is an example of the amount of sequence modified CGRP to include in a transdermal delivery system that will deliver sequence modified CGRP across the skin at a rate suitable to maintain a steady state plasma level of 157±26 μg/mL, which has been found to produce profoundly beneficial hemodynamic responses including increased cardiac output, decreased ventricular filling pressures, pulmonary and systemic arterial pressures, vascular resistance, increased glomerular filtration, and renal blood flow. If it is assumed that a transdermal delivery system can deliver 25% of the loaded sequence modified CGRP across the skin, then in order to deliver a total drug load similar to that delivered by an IV dose of 560 ng/min (0.008 μg/kg/min) over 8-24 hours (i.e., delivery of 288-806 μg sequence modified CGRP) the total drug load required for the transdermal delivery system would be approximately 1.152-3.456 mg. Polymer matrix systems capable of delivering 100% of the drug at a rate suitable to maintain similar steady state plasma levels would require a total drug load four times less than transdermal systems, i.e., 0.288-0.806 mg. Peak plasma levels of sequence modified CGRP at 157±26 pg/ml are obtained in the first 60 minutes. In a preferred embodiment the peak level is maintained for 8-24 hours.

TABLES 4 and 5 show examples of the amount of sequence modified CGRP to be added to a flowable composition and the corresponding injection volumes in order to produce implants that will provide the indicated delivery rates over 7, 30, 60, 90, 120 or 180 days and maintain steady state plasma levels of sequence modified CGRP up to 157±26 pg/mL. In TABLES 4 and 5, delivery rates and sequence modified CGRP loads are provided for compositions that will produce implants in situ comprising 5 wt. % and 15 wt. % sequence modified CGRP, respectively.

TABLE 4
Thermoplastic Polymer Compositions Comprising
a Loading of 5% Sequence Modified CGRP

| | Delivery Duration of Release (Days) | | | | | |
|---|---|---|---|---|---|---|
| Delivery Rate | | 7 | 30 | 90 | 120 | 180 |
| 0.0008 μg/kg/min | Drug Load (mg) | 0.56 | 2.42 | 7.26 | 9.68 | 14.52 |
| | Injection (cc) | 0.01 | 0.05 | 0.15 | 0.19 | 0.29 |
| 0.0032 μg/kg/min | Drug Load (mg) | 2.26 | 9.69 | 29.07 | 38.76 | 58.14 |
| | Injection (cc) | 0.04 | 0.19 | 0.58 | 0.77 | 1.16 |
| 0.008 μg/kg/min | Drug Load (mg) | 5.64 | 24.18 | 72.54 | 96.72 | 145.08 |
| | Injection (cc) | 0.11 | 0.48 | 1.45 | 1.93 | 2.90 |
| 0.016 μg/kg/min | Drug Load (mg) | 11.27 | 48.30 | 144.90 | 193.20 | 289.80 |
| | Injection (cc) | 0.22 | 0.97 | 2.90 | 3.86 | 5.80 |
| 0.024 μg/kg/min | Drug Load (mg) | 16.91 | 72.48 | 217.44 | 289.92 | 434.88 |
| | Injection (cc) | 0.33 | 1.45 | 4.35 | 5.79 | 8.7 |

TABLE 5
Thermoplastic Polymer Compositions Comprising
a Loading of 15% Sequence Modified CGRP

| | Delivery Duration of Release (Days) | | | | | |
|---|---|---|---|---|---|---|
| Delivery Rate | | 7 | 30 | 90 | 120 | 180 |
| 0.0008 μg/kg/min | Drug Load (mg) | 0.56 | 2.42 | 7.26 | 9.68 | 14.52 |
| | Injection (cc) | 0.004 | 0.02 | 0.05 | 0.065 | 0.097 |
| 0.0032 μg/kg/min | Drug Load (mg) | 2.26 | 9.69 | 29.07 | 38.76 | 58.14 |
| | Injection (cc) | 0.02 | 0.07 | 0.19 | 0.26 | 0.39 |
| 0.008 μg/kg/min | Drug Load (mg) | 5.64 | 24.18 | 72.54 | 96.72 | 145.08 |
| | Injection (cc) | 0.04 | 0.16 | 0.48 | 0.64 | 0.97 |
| 0.016 μg/kg/min | Drug Load (mg) | 11.27 | 48.30 | 144.90 | 193.20 | 289.80 |
| | Injection (cc) | 0.08 | 0.32 | 0.97 | 1.29 | 1.93 |
| 0.024 μg/kg/min | Drug Load (mg) | 16.91 | 72.48 | 217.44 | 289.92 | 434.88 |
| | Injection (cc) | 0.12 | 0.48 | 1.45 | 1.93 | 2.9 |

For example, in one embodiment of the present invention, a polymeric composition comprising 5 wt. % sequence modified CGRP (i.e., 5.64 mg sequence modified CGRP) can be formulated to produce a polymer matrix in situ that will deliver sequence modified CGRP at circulating plasma levels of sequence modified CGRP up to 157±26 pg/mL or deliver sequence modified CGRP at a rate of 0.008 μg g/kg/min over a period of 7 days when about 0.11 mL of this composition is administered to a patient (Table 4). Alternatively, if it is desired to have the sequence modified CGRP delivered at circulating plasma levels of sequence modified CGRP of 157±26 pg/mL or a rate of 0.008 μg/kg/min over a period of 180 days, a composition comprising 15 wt. % sequence modified CGRP (i.e., 145.08 mg sequence modified CGRP) can be prepared and about 0.97 mL of this composition is administered to the patient (Table 5). In a similar fashion, other compositions can be prepared according to the examples shown in Tables IV and V to provide the desired circulating plasma levels of sequence modified CGRP and delivery rate over the targeted time period. It is to be understood that the formulations in Tables IV and V are provide as examples to illustrate the invention, and it would be well within the skill of persons of ordinary skill in the art to design other formulations that would yield different delivery rates over different time periods.

The compositions of this invention can be delivered directly to a target site and can be designed to provide continuous release of sequence modified CGRP over a targeted time period so as to reduce the frequency of drug administration. In general, a solid implant or matrix is formed upon dispensing the flowable polymeric composition either into a tissue or onto the surface of a tissue, which is surrounded by an aqueous medium. The composition can be delivered to a patient's tissue or bodily fluid by any convenient technique. For example, the thermoplastic polymeric solution can be placed in a syringe and injected through a needle into a patient's body, i.e., in the desired tissue site or bodily fluid. Upon discharge of the composition from the needle into the tissue or fluid, the solvent dissipates or diffuses away from the polymer and into the surrounding fluid, resulting in the precipitation of the biocompatible polymer, which precipitate forms a coherent mass or polymer matrix. The polymer matrix can adhere to its surrounding tissue or bone by mechanical forces and can assume the shape of its surrounding cavity and conform to the irregular surface of the tissue. The implant will biodegrade over time and does not require removal when sequence modified CGRP is depleted.

In certain instances, formation of the solid matrix from the flowable delivery system is not instantaneous. For example, the process can occur over a period of minutes to several hours. During this period, the rate of diffusion of sequence modified CGRP from the coagulating polymeric composition may be much more rapid than the rate of release that occurs from the subsequently formed solid matrix. “Initial burst” refers to the release of a sequence modified CGRP from the polymeric composition during the first 24 hours after the polymeric composition is contacted with an aqueous fluid. This initial “burst” of sequence modified CGRP that is released during polymer matrix formation may result in the loss or release of a large amount of the active agent. Therefore, in certain embodiments the thermoplastic polymer composition can further comprise a polymeric controlled release additive that substantially reduces the “initial burst” of sequence modified CGRP released from the polymeric composition during the initial 24 hours after implantation. The use of such an additive is described in U.S. Pat. No. 6,143,314, which is specifically incorporated herein by reference. As used herein, the term “substantially reduces” means a decrease of at least 15%, and preferably between about 15% to about 70%, of sequence modified CGRP that is released from the polymeric composition compared to a composition without the additive. Examples of suitable controlled release additives include thermoplastic polymers having poly(lactide-co-glycolide) (PLG) moieties and polyethylene glycol (PEG) moieties. In one embodiment, the controlled release additive is a poly(lactide-co-glycolide)/polyethylene glycol (PLG/PEG) block copolymer. The polymeric controlled release additive is present in the polymeric composition in an amount effective to reduce the initial burst of biologically active agent released from the polymeric composition during the first 24 hours after implantation. Preferably, the polymeric composition includes about 1 wt. % to about 50 wt. %, more preferably about 2 wt. % to about 20 wt. % of the polymeric controlled release additive.

The solid matrix is capable of biodegradation, bioerosion, and/or bioabsorption within the implant site of the patient or animal, and will slowly biodegrade within the body and will release sequence modified CGRP contained within its matrix at a controlled rate until depleted. Generally, the polymer matrix will breakdown over a period from about 1 week to about 12 months and can be adjusted by one skilled in the art of biodegradable polymer drug delivery. The release of sequence modified CGRP can be affected by the size and shape of the polymer matrix, the loading of drug within the polymer matrix, the permeability factors involving sequence modified CGRP and the particular polymer, and the degradation of the polymer. The above parameters can be adjusted by one skilled in the art of drug delivery to give the desired rate and duration of release (see for example Tables IV and V).

The polymeric sequence modified CGRP solution can be placed anywhere within the body, including tissue sites such as soft tissue (e.g., muscle or fat), hard tissue (e.g., bone), or a cavity such as the periodontal, oral, vaginal, rectal, or nasal cavity. As used herein, the term “tissue site” includes any tissues in an organism. A tissue site is typically surrounded by an aqueous or body fluid such as subcutaneous tissue, interstitial fluid, blood, serum, cerebrospinal fluid or peritoneal fluid.

A suitable polymeric gel for use in this embodiment comprises ABA- or BAB-type block copolymers, where the A-blocks are relatively hydrophobic A polymer blocks comprising a biodegradable polyester, and the B-blocks are relatively hydrophilic B polymer blocks comprising polyethylene glycol (PEG). The A block is-preferably a biodegradable polyester synthesized from monomers selected from the group consisting of D,L-lactide, D-lactide, L-lactide, D,L-lactic acid, D-lactic acid, L-lactic acid, glycolide, glycolic acid, .epsilon.-caprolactone, .epsilon.-hydroxyhexanoic .lamda.-hydroxybutyric acid, .delta.-valerolactone, .delta.-hydroxyvaleric acid, hydroxybutyric acids, malic acid, and copolymers thereof, and the B block is PEG. The polymeric gel is preferably biodegradable and exhibits water solubility at low temperatures and undergoes reversible thermal gelation at physiological mammalian body temperatures. Furthermore, these polymeric gels are biocompatible and capable of releasing sequence modified CGRP entrained within its matrix over time and in a controlled manner. The polymeric gel may be prepared as disclosed in U.S. Pat. No. 6,201,012, which is incorporated herein by reference.

Other suitable polymers include in situ formed hydrogels prepared from thermosensitive block copolymers. Such block copolymers undergo reversion between gel and sol under certain conditions. The gel-sol transition temperature is generally above room temperature, which depends on the composition of the gel, as well as on the chemical structure and molecular weight of PEG or PEG copolymers. The polymer is a poly(ethylene glycol), a derivative thereof, or a copolymer that reacts with the poly(ethylene glycol) segment. The polymer can also be poly(propylene glycol) (PPG) or other poly(alkylene glycols). Higher molecular weight poly(ethylene glycol) is also called poly(ethylene oxide) (PEO). Poly(ethylene glycol) block copolymers with poly(propylene oxide) (PPO), including an pluronic polymers (Poloxamers) may also be used. Different molecular weight of each segment, and weight ratio of the blocks, and different sequences may be used such as PEO-PPO-PEO (Pluronic), PPO-PEO-PPO (Pluronic-R), PEO-PPO, etc.

Suitable polymers useful in the invention include PLURONIC (BASF Corp.) surfactant which is a group of poly(ethylene oxide)-polypropylene oxide)poly(ethylene oxide) triblock copolymers also known as poloxamers. The PEG block at both ends is able to complex with alpha-cyclodextrin, just like the PEG molecules. PLURONIC polymers have unique surfactant abilities and extremely low toxicity and immunogenic responses. These products have low acute oral and dermal toxicity and low potential for causing irritation or sensitization, and the general chronic and subchronic toxicity is low. In fact, PLURONIC polymers are among a small number of surfactants that have been approved by the FDA for direct use in medical applications and as food additives (BASF (1990) Pluronic & Tetronic Surfactants, BASF Co., Mount Olive, N.J.). Recently, several PLURONIC polymers have been found to enhance the therapeutic effect of drugs (March, K. L., et al., Hum. Gene Therapy 6(1): 41-53, 1995).

The hydrogel-based injectable composition may be prepared in any suitable manner. Generally, sequence modified CGRP in aqueous solution is combined with the poly(ethylene glycol) component. The mixture is cooled, generally to a temperature of 0° C. to 25° C. The resulting product is a white viscous hydrogel.

### Controlled Release Films

This invention further provides a prophylaxis for a method of treatment comprising administering a biodegradable, biocompatible polymeric film comprising sequence modified CGRP including corresponding retro-inverso forms to a patient. The polymeric films are thin compared to their length and breadth. The films typically have a uniform selected thickness between about 60 micrometers and about 5 mm. Films of between about 600 micrometers and 1 mm and between about 1 mm and about 5 mm thick, as well as films between about 60 μm and about 1000 μm; and between about 60 μm and about 300 μm are useful in the manufacture of therapeutic implants for insertion into a patient's body. The films can be administered to the patent in a manner similar to methods used in adhesion surgeries. For example, a sequence modified CGRP film formulation can be sprayed or dropped onto a tissue site during surgery, or a formed film can be placed over the selected tissue site. In an alternative embodiment, the film can be used as sustained release coating on a medical device such as a stent.

Either biodegradable or non-biodegradable polymers may be used to fabricate implants in which the sequence modified CGRP is uniformly distributed throughout the polymer matrix. A number of suitable biodegradable polymers for use in making the biodegradable films of this invention are known to the art, including polyanhydrides and aliphatic polyesters, preferably polylactic acid (PLA), polyglycolic acid (PGA) and mixtures and copolymers thereof, more preferably 50:50 copolymers of PLA:PGA and most preferably 75:25 copolymers of PLA:PGA. Single enantiomers of PLA may also be used, preferably L-PLA, either alone or in combination with PGA. Polycarbonates, polyfumarates and caprolactones may also be used to make the implants of this invention.

A plasticizer may be incorporated in the biodegradable film to make it softer and more pliable for applications where direct contact with a contoured surface is desired.

The polymeric films of this invention can be formed and used as flat sheets, or can be formed into three-dimensional conformations or “shells” molded to fit the contours of the tissue site into which the film is inserted.

To make the polymeric films of this invention, a suitable polymeric material is selected, depending on the degradation time desired for the film. Selection of such polymeric materials is known to the art. A lower molecular weight, e.g. around 20,000 daltons, 50:50 or 55:45 PLA:PGA copolymer is used when a shorter degradation time is desired. To ensure a selected degradation time, the molecular weights and compositions may be varied as known to the art.

Polymeric films of this invention may be made by dissolving the selected polymeric material in a solvent known to the art, e.g., acetone, chloroform or methylene chloride, using about 20 mL solvent per gram of polymer. The solution is then degassed, preferably under gentle vacuum to remove dissolved air and poured onto a surface, preferably a flat non-stick surface such as BYTAC™ (Norton Performance Plastics, Akron, Ohio) non-stick coated adhesive-backed aluminum foil, glass or TEFLON™ non-stick polymer. The solution is then dried, preferably air-dried, until it is no longer tacky and the liquid appears to be gone. The known density of the polymer may be used to back-calculate the volume of solution needed to produce a film of the desired thickness.

Films may also be made by heat pressing and melt forming/drawing methods known to the art. For example, thicker films can be pressed to form thinner films, and can be drawn out after heating and pulled over forms of the desired shapes, or pulled against a mold by vacuum pressure.

The amount of sequence modified CGRP to be incorporated into the polymeric films of this invention is an amount effective to show a measurable effect in treatment (e.g. preventing or treating HF, MI, angina, vasospasm, and/or renal failure). Sequence modified CGRP can be incorporated into the film by various techniques such as by solution methods, suspension methods, or melt pressing.

Solid sequence modified CGRP implants can also be made into various shapes other than films by injection molding or extrusion techniques. For example, the implant can comprise a core material such as ethylene/vinyl acetate copolymer, and a vinyl acetate content of 20% by weight or more and which functions as a matrix for sequence modified CGRP, in a quantity which is sufficient for a controlled release of sequence modified CGRP, and a membrane which encases the core material and also consists of EVA material and an acetate content of less than 20% by weight. The implant can be obtained, for example, by means of a co-axial extrusion process, a method in which the two EVA polymers are extruded co-axially with the aid of a co-axial extrusion head. The co-axial extrusion process is art known per se so that it will not be gone into further within the scope of this description.

### Encapsulated Sequence Modified CGRP

Yet another sequence modified CGRP controlled release formulation according to this invention comprises very small capsules which can be administered, for example by injection, into body tissue of fluids. Accordingly, this invention further provides a method of treatment comprising by administering capsules comprising sequence modified CGRP, and a kit comprising the capsules. The capsules include an encapsulating layer which surrounds sequence modified CGRP or comprises sequence modified CGRP dispersed throughout the encapsulating layer. After injection, the -encapsulating layer degrades or dissolves, and sequence modified CGRP is released within the heart. Sequence modified CGRP can also diffuse through the encapsulating layer. The encapsulating layer may be made from various materials including biodegradable polymers in the form of microspheres, or from standard vesicle forming lipids which form liposomes and micelles. Such sustained release sequence modified CGRP capsules are useful for treatment or prophylaxis of, for example, HF, MI, angina, vasospasm, renal failure, pulmonary hypertension, and/or sexual dysfunction. Both biodegradable and nonbiodegradable polymers may be used to prepare formulations in which sequence modified CGRP is encapsulated within a polymer matrix and surrounded by a rate-controlling membrane.

Microparticles: One embodiment of controlled release, sequence modified encapsulated CGRP, comprises solid microparticles formed of the combination of biodegradable polymers incorporating sequence modified CGRP loadings that yield a sustained release over a period of one day to at least one week, when administered orally, transmucosally, topically, or by injection. In delivery systems incorporating such microparticles, the microparticles have different diameters depending on their route of administration. For example, microparticles administered by injection have diameters sufficiently small to pass through a needle, in a size range of between 10 and 100 μm. Orally administered microparticles are preferably less than 10 μm in diameter to facilitate uptake by the small intestine. The microparticles can contain from less than 0.01% by weight up to approximately 50% by weight sequence modified CGRP. Pharmaceutical compositions incorporating microparticle delivery systems containing sequence modified CGRP can be combined with pharmaceutical excipients, diluents, or carriers.

As used herein, “micro” refers to a particle having a diameter of from nanometers (nm) to micrometers (μm). Microspheres are solid spherical particles; microparticles are particles of irregular or non-spherical shape. A microparticle may have an outer coating of a different composition than the material originally used to form the microsphere. Thus, the term “microparticle” as used herein encompasses microparticles, microspheres, and microcapsules.

Polymers that can be used to form the microparticles include, but are not limited to, biodegradable polymers such as poly(lactic-co-glycolic acid) (PLG), poly(lactic acid) (PLA), poly(caprolactone), polycarbonates, polyamides, polyanhydrides, polyamino acids, polyortho esters, polyacetals, polycyanoacrylates, degradable polyurethanes, polyacrylates, ethylene-vinyl acetate copolymers, acyl substituted cellulose acetates, and derivatives and copolymers thereof. Almost any type of polymer can be used provided the appropriate solvent and non-solvent are found which have the desired melting points. Pharmaceutical compositions and delivery systems can be prepared incorporating microparticles prepared from these polymers.

Biodegradable microspheres can be prepared using any of the methods developed for making microspheres for drug delivery, for example, as described by Mathiowitz and Langer (J. Controlled Release, 5:13-22 (1987)); Mathiowitz, et al. (Reactive Polymers, 6:275-283 (1987)); and Mathiowitz, et al. (J. Appl. Polymer Sci., 35:755-774 (1988)), the teachings of which are incorporated herein. The selection of the method depends on the polymer selection, the size, external morphology, and crystallinity that is desired, as described, for example, by Mathiowitz, et al. (Scanning Microscopy, 4:329-340 (1990)); Mathiowitz, et al. (J. Appl. Polymer Sci., 45:125-134 (1992)); and Benita, et al. (J. Pharm. Sci., 73:1721-1724 (1984)), the teachings of which are incorporated herein. Methods include solvent evaporation, phase separation, spray drying, and hot melt encapsulation. U.S. Pat. Nos. 3,773,919; 3,737,337; 3,523,906; 4,272,398; 5,019,400; 5,271,961, and 6,403,114 are representative of methods for making microspheres, each of which is specifically incorporated herein by reference. U.S. Pat. No. 5,019,400, which is incorporated herein by reference, describes the Prolease™ process in which microspheres can be formed in a size suitable for injection through a 26-gauge needle, (less than 50 micrometers in diameter). The process described in U.S. Pat. No. 5,019,400 has the advantage of achieving drug encapsulation in the absence of water at very low temperatures. These conditions are particularly suitable for fragile macromolecules such as proteins, where maintaining stability is a concern. Microparticles can be formed by either a continuous freezing and extraction process or by a batch process wherein a batch of frozen microdroplets is formed in a first step, and then in a separate second step, the frozen microdroplets in the batch are extracted to form microparticles. U.S. Pat. No. 6,403,114 describes a method of preparing microspheres in commercial batch sizes, and U.S. Pat. No. 5,271,961 describes a continuous method of preparing microspheres. Each of these patents are incorporated herein by reference

In general, microspheres incorporating sequend modified CGRP can be prepared by combining sequence modified CGRP, the polymer, and a solvent to form a droplet, and then removing the solvent to yield microspheres that are hardened, dried, and collected as a free-flowing powder. Prior to administration to the patient, the powder is suspended in a diluent and then injected into the patient. Release of sequence modified CGRP from the microsphere is governed by diffusion of sequence modified CGRP through the polymer matrix and by biodegradation of the polymer. The release kinetics can be modulated through a number of formulation and fabrication variables including polymer characteristics and the addition of excipients and release modifiers. In solvent evaporation, described in U.S. Pat. No. 4,272,398, which is incorporated herein by reference, the polymer is dissolved in a volatile organic solvent. The sequence modified CGRP, either in soluble form or dispersed as fine particles, is added to the polymer solution, and the mixture is suspended in an aqueous phase that contains a surface active agent such as poly(vinyl alcohol). The resulting emulsion is stirred until most of the organic solvent evaporates, leaving solid microspheres. After loading the solution with sequence modified CGRP, the solution is suspended in distilled water containing 1% (w/y) poly(vinyl alcohol), after which the solvent is evaporated and resulting microspheres are dried overnight in a lyophilizer. Microspheres with different sizes (1-1000 μm) and morphologies can be obtained by this method which is useful for relatively stable polymers such as polyesters and polystyrene.

Polymer hydrolysis is accelerated at acidic or basic pH and thus the inclusion of acidic or basic excipients can be used to modulate the polymer erosion or degradation rate. The excipients can be added as particulates, can be mixed with the incorporated sequence modified CGRP or can be dissolved within the polymer.

Degradation modulators can also be added to the microparticle formulation, and the amount added is based on weight relative to the polymer weight. They can be added to the formulation as a separate phase (i.e., as particulates) or can be codissolved in the polymer phase depending on the compound. In all cases the amount of enhancer added is preferably between 0.1 and thirty percent (w/w, polymer). Types of degradation modulators include inorganic acids such as ammonium sulfate and ammonium chloride, organic acids such as citric acid, benzoic acids, heparin, and ascorbic acid, inorganic bases such as sodium carbonate, potassium carbonate, calcium carbonate, zinc carbonate, and zinc hydroxide, and organic bases such as protamine sulfate, sperinine, choline, ethanolamine, diethanolamine, and triethanolamine and surfactants such as Tween™ and Pluronic™.

Stabilizers can be also added to the formulations to maintain the potency of sequence modified CGRP depending on the duration of release. Stabilizers include carbohydrates, amino acids, fatty acids, and surfactants and are known to those skilled in the art. In addition, excipients which modify the solubility of sequence modified CGRP such as salts complexing agents (albumin, protamine) can be used to control the release rate of the protein from the microparticles.

In one embodiment, the patient is administered sequence modified CGRP incorporated in microparticles which degrade over a period of 1 of 2 months. The microparticles preferably range in size from 10 to 60 and can be injected using a puncture needle with the aid of a suspension media. One example of a suspension media comprises 3% methyl cellulose, 4% mannitol, and 0.1% Tween™ 80.

In a further embodiment, microparticles containing sequence modified CGRP can be embedded in a gel matrix as described in U.S. Pat. No. 6,589,549, which is incorporated herein by reference. In this embodiment, sequence modified CGRP (alone or in combination with one or more additional agents) may be located in the microparticle alone or both in the microparticle and the gel matrix. The microparticle-gel delivery system can release sequence modified CGRP over a prolonged period of time at a relatively constant rate. The release profile of the system can be modified by altering the microparticle and/or the gel composition. After injection, the gel sets and localizes the microparticle suspended in it. Sequence modified CGRP encapsulated in the microparticle must be released from the microparticle before traveling through the gel matrix and entering the biological system. Therefore, the immediate release, or the burst, associated with microparticle delivery systems can be reduced and modulated. Since the release rates of sequence modified CGRP from these two systems can be quite different, embedding microparticles in the gel phase offers additional modulation and economical use of sequence modified CGRP. The benefits include higher bioavailability and longer duration of action than either system when used alone. Moreover, the combined system can improve the safety of microparticle dosage form. Microparticles containing sequence modified CGRP embedded in a gel matrix can be used to prepare pharmaceutical compositions.

A suitable polymeric gel for use in this embodiment comprises ABA- or BAB-type block copolymers, where the A-blocks are relatively hydrophobic A polymer blocks comprising a biodegradable polyester, and the B-blocks are relatively hydrophilic B polymer blocks comprising polyethylene glycol (PEG). The A block is preferably a biodegradable polyester synthesized from monomers selected from the group consisting of D,L-lactide, D-lactide, L-lactide, D,L-lactic acid, D-lactic acid, L-lactic acid, glycolide, glycolic acids ε-caprolactone, .ε-hydroxyhexanoic λ-hydroxybutyric acid, δ-valerolactone, δ-hydroxyvaleric acid, hydroxybutyric acids, malic acid, and copolymers thereof, and the B block is PEG. The polymeric gel is preferably biodegradable and exhibits water solubility at low temperatures and undergoes reversible thermal gelation at physiological mammalian body temperatures. Furthermore, these polymeric gels are biocompatible and capable of releasing sequence modified CGRP entrained within its matrix over time and in a controlled manner. The polymeric gel may be prepared as disclosed in U.S. Pat. No. 6,201,072, which is incorporated herein by reference.

### Solid Implants

Solid implants made by injection molding or extrusion methods similar to that used to manufacture Norplant™, a product brand and a trademark of Leiras Co., which is based on a non-degradable polymeric material. In this embodiment, a definitely formed, device constructed of silicone rubber which is implanted into the body by a surgical operation, and it is removed therefrom in a similar manner after a defined time when the active component has been released and diffused to the body. Any of the polymeric materials utilized for the construction of implantable devices may be used in the practice of the invention. A broad class of silicone elastomers can be used to form the silicone-elastomer drug matrix. Suitable silicone elastomers in accordance with the present invention include SILASTIC™ and R-2602 RTV silicone elastomer available from Nusil Silicone Technology (Carpinteria, Calif.). The silicone elastomers can be catalyzed so that polymerization and formation of the core is accomplished at room temperature. The core may also be formed by heat curable core material. Generally, the silicone implantable depots are constructed of polydimethylsilicone (PDMS). See, for example, U.S. Pat. Nos. 4,957,119 and 5,088,505, which are incorporated herein by reference. A typical material is dimethylpolysiloxane (Silgel™ 601, Wacker Chemie GmbH), an addition cross-linking two-component composition of nine pats of component A and one part of component B. Dimethyldiphenylpolysiloxane, dimethylpolysiloxanol or silicone copolymers may also be employed. Other suitable polymeric materials are the porous, ethylene/vinyl acetate copolymers which have been utilized to construct depots for the implantable release of hydrophilic biologically active substances such as proteins through the pores thereof. Biodegradable polymers may also be used to form the solid implants using extrusion or injection molding processes.

### Liposomes

Another method of delivering sequence modified CGRP to a patient is accomplished with encapsulation by liposomes, wherein sequence modified CGRP may be sequestered in the liposome membrane or may be encapsulated in the aqueous interior of the vesicle. The term “liposome” refers to an approximately spherically shaped bilayer structure, or vesicle, comprised of a natural or synthetic phospholipid membrane or membranes that contain two hydrophobic tails consisting of fatty acid chains, and sometimes other membrane components such as cholesterol and protein, which can act as a physical reservoir for sequence modified CGRP. Upon exposure to water, the phospholipid molecules spontaneously align to form spherical, bilayer membranes with the lipophilic ends of the molecules in each layer associated in the center of the membrane and the opposing polar ends forming the respective inner and outer surface of the bilayer membrane(s). Thus, each side of the membrane presents a hydrophilic surface while the interior of the membrane comprises a lipophilic medium. These membranes may be arranged in a series of concentric, spherical membranes separated by thin strata of water around an internal aqueous space. These multilamellar vesicles (MLV) can be converted into small or unilamellar vesicles (UV), with the application of a shearing force. Liposomes are characterized according to size and number of membrane bilayers. Vesicle diameters can be large (>200 nm) or small (<50 nm) and the bilayer can have unilamellar, oligolamellar, or multilamellar membrane.

The selection of lipids is generally guided by considerations of liposome size and ease of liposome sizing, and lipid and sequence modified CGRP release rates from the site of liposome delivery. Typically, the major phospholipid components in the liposomes are phosphatidylcholine (PC), phosphatidylglycerol (PG), phosphatidyl serine (PS), phosphatidylinositol (PI) or egg yolk lecithin (EYL). PC, PG, PS, and PI having a variety of acyl chains groups or varying chain lengths are commercially available, or may be isolated or synthesized by known techniques.

Current methods of drug delivery by liposomes require that the liposome carrier will ultimately become permeable and release the encapsulated drug. This can be accomplished in a passive manner in which the liposome membrane degrades over time through the action of agents in the body. Every liposome composition will have a characteristic half-life in the circulation or at other sites in the body. In contrast to passive drug release, active drug release involves using an agent to induce a permeability change in the liposome vesicle. In addition, liposome membranes can be made which become destabilized when the environment becomes destabilized near the liposome membrane (Proc. Nat. Acad. Sci., 84:7851 (1987); Biochemistry, 28:9508, (1989)). For example, when liposomes are endocytosed by a target cell they can be routed to acidic endosomes which will destabilize the liposomes and result in drug release. Alternatively, the liposome membrane can be chemically modified such that an enzyme is placed as a coating on the membrane which slowly destabilizes the liposome (The FASEB Journal, 4:2544 (1990)). It is also well known that lipid components of liposomes promote peroxidative and free radical reactions which cause progressive degradation of the liposomes, and has been described in U.S. Pat. No. 4,797,285. The extent of free radical damage can be reduced by the addition of a protective agent such as a lipophilic free radical quencher is added to the lipid components in preparing the liposomes. Such protectors of liposome are also described in U.S. Pat. No. 5,190,761, which also describes methods and references for standard liposome preparation and sizing by a number of techniques. Protectors of liposomal integrity will increase the time course of delivery and provide for increased transit time within the target tissue.

Liposomes for use in the present invention can be prepared by any of the various techniques presently known in the art. Typically, they are prepared from a phospholipid, for example, distearoyl phosphatidylcholine, and may include other materials such as neutral lipids, for example, cholesterol, and also surface modifiers such as positively charged (e.g., sterylamine or aminomannose or aminomannitol derivatives of cholesterol) or negatively charged (e.g., diacetyl phosphate, phosphatidyl glycerol) compounds. Multilamellar liposomes can be formed by conventional techniques, that is, by depositing a selected lipid on the inside wall of a suitable container or vessel by dissolving the lipid in an appropriate solvent, and then evaporating the solvent to leave a thin film on the inside of the vessel or by spray drying. An aqueous phase is then added to the vessel with a swirling or vortexing motion which results in the formation of MLVs. UVs can then be formed by homogenization, sonication or extrusion (through filters) of MLV's. In addition, UVs can be formed by detergent removal techniques

Liposomes containing sequence modified CGRP can be used to prepare pharmaceutical compositions. The liposomes containing sequence modified CGRP can be delivered within biodegradable microdrug delivery systems such as larger more stable liposomes or other fully encapsulated controlled release system, such as a biodegradable impermeable polymer coatings. The time course of release is governed then by the additive time delay of the barriers that separate sequence modified CGRP from the host, as well as their combined transport pathways. Microparticle delivery systems may also be used.

### Topical Administration

Once the desired sequence modified CGRP is obtained, chemical conjugation to a hydrophobic agent may proceed thereby forming a hydrophobic-sequence modified CGRP for topical use. Such conjugates can be used in topical, skin-penetrable pharmaceutical compositions.

In one embodiment, the sequence modified CGRP is conjugated to a naturally occurring polyunsaturated fatty acid, such as linolenic acid. Acetoxymethyl acetate or acetoxymethyl esters can also be used for this purpose instead of linolenic acid. This sequence modified CGRP ester (conjugate) may be prepared using an automated peptide synthesizer and known methods. Alternately, the sequence modified CGRP ester may be prepared by reacting the sequence modified CGRP and linolenic acid using carbodiimide, glutaraldehyde, or a similar compounds, as a coupling agent.

In the case of conjugation of sequence modified CGRP to a fatty acid manually, a 1:1 weight ratio of sequence modified CGRP should be allowed to react with citraconic anhydride at a pH of 8.5 (to block free amine groups) while mixing with a magnetic stir bar. After 60 minutes at room temperature, the blocked peptide should be separated from other free compounds by a G10 gel-permeation column. The blocked peptide then should be allowed to react with the same weight of coupling reagent and the pH should be adjusted to 8.0. The mixture should be incubated for 10 minutes and an equal volume of fatty acid—preferably linolenic acid, C₁₈H₃₀O₂. (MW 278.4). In molar proportions, about 50 mol of fatty acid should be added for every 1 mol of peptide. After four hours at room temperature, 100 mmol/L of sodium acetate (pH 4.2) should be added to terminate the reaction. The resultant material should be dialyzed to remove the sodium acetate with 5 changes of buffer. The material then should be dialyzed overnight against phosphate buffered saline (pH 7.4) to remove all uncoupled reagents. This last dialysis step (i.e., separation of the conjugated compound) also may be achieved by gel-permeation chromatography.

Alternatively, hydrophobic-sequence modified CGRP may be prepared by linking the N-terminal amino group of sequence modified CGRP to the C-terminal carboxyl group via (acyloxy) alkoxy promoiety Boc. In this convergent approach, this pro-peptide is reacted with 1-chloromethyl chloroformate with p-nitrophenol in the presence of N-methylmorpholine (NMM) to afford 1-chloromethyl)-p-nitrophenyl carbonate. Substitution of the chloride can be achieved with iodide, leading to iodo compounds with high yield. The resultant compound is then reacted with the cesium salt of Boc-Ala in dimethyl-formamide (DMF) to give a mixture of the desired product Boc-(alaninyloxy)methyl p-nitrophenyl carbonate, and the side product of p-nitrophenyl ester of Boc-alanine (Boc-Ala-OpNP). This mixture then couples with Trp-Obzl in the presence of NMM and 1-hydroxybenzotri-azole (HOBT) in hexamethylphos-phoramide (HMPA) to afford the Boc-[[(alaninyloxy)methyl]-carbonyl]-N-tryptophan benzyl ester and the side product of Boc-Ala-Trp-OBzl. Hydrogenolysis of this mixture using 10% Pd/C as catalyst under the H₂ atmosphere in EtOH will give over 95% pure compound of interest. The active compound is then purified from the side product Boc-Ala-Trp-OH by reverse-phased high performance liquid chromatography. In vivo, this cyclic pro-peptide will release linear sequence modified CGRP, which is biologically active.

Once the sequence modified CGRP ester is formed, the sequence modified CGRP ester should be mixed with a pharmaceutical base cream such as a lipophilic or aqueous based cream to result in a concentration of 2.5 nmol/dose (10.18 μg/dose). A preferred cream is a lipophilic base such as but not limited to Aquaphore™. Various additives included in the composition of the present invention include but are not limited to flavoring agents, Vitamin E, L-Arginine, -Valine, peppermint oil, ginger oil, cinnamon oil, Gynostemma pentaphyllum, Crataegus pinnatifidia, Yohimbine, and prostaglandin E₁, (PGE₁). Other additives such as, but not limited to ethylene oxide and propylene oxide block copolymers (pluronics) that are widely known as agents that promote drug penetration across biological barriers can further be added. Pluronics L61 and P85 that have different hydrophobicity are examples of such block copolymers.

The compounds and/or compositions of the invention can be administered by any available and effective delivery systems including, topical application or transdermally, in dosage unit formulations containing conventional nontoxic pharmaceutical carriers, adjuvants, and vehicles, as desired. The route of administration is at the discretion of the physician who takes into consideration the condition of the patient undergoing treatment.

After topical application of about 0.1-0.5 ml of the sequence modified CGRP cream and gentle rubbing, the “fatty acid-sequence modified CGRP ester” will readily penetrate through the skin and accumulate. Naturally occurring esterases in the subcutaneous tissues will then release active sequence modified CGRP into the local environment from the fatty acid conjugate. The result is rapid and sustained vasodilation of the blood vessels. The effect of the sequence modified CGRP should last between about 20-45 minutes. The local application may be repeated, if necessary, to result in continued vasodilation lasting about 20-45 minutes. Persons of skill in the art can adjust the foregoing parameters to locally apply sequence modified CGRP to specific sites in the body.

Formulations of hydrophobic-sequence modified CGRP, sequence modified CGRP or fragment thereof can conveniently be presented in unit dosage forms and can be prepared by any of the methods known in the pharmaceutical arts. Transdermal drug administration, which is known to one skilled in the art, involves the delivery of pharmaceutical agents via percutaneous passage of the drug into the systemic circulation of the patient. Topical administration can also involve transdermal patches or iontophoresis devices. Other components can also be incorporated into the transdermal patches. For example, compositions and/or transdermal patches can be formulated with one or more preservatives or bacteriostatic agents including, for example, methyl hydroxybenzoate, propyl hydroxybenzoate, chlorocresol, benzalkonium chloride, and the like.

Dosage forms for topical administration of the compounds and/or compositions of the invention can include creams, sprays, lotions, gels, ointments, and the like. Administration of the cream or gel can be accompanied by use of an applicator or vaginal insert or device, and is within the skill of the art. Typically, any pharmaceutical preparation may be used, in particular a preferred cream being Aquaphore, which is commercially available from Beiersdorf Inc., (Norwalk, Conn.). The concentration of sequence modified CGRP in the cream should range from about 1-3 nmol/dose (4.07-12.22 μg/dose), and in one preferred embodiment be about 2.5 nmol/dose (10.18 μg/dose). A lubricant can also be included in the formulation or provided for use as needed. Lubricants include, for example, K-Y jelly (available from Johnson & Johnson) or a lidocaine jelly, such as Xylocaine 2% jelly (available from Astra Pharmaceutical Products).

The compounds and/or compositions of the invention will typically be administered in a pharmaceutical composition containing one or more selected carriers or excipients. Suitable carriers include, for example, water, silicone, waxes, petroleum jelly, polyethylene glycol, propylene glycol, liposomes, transfersomes, vitamin E, sugars, and the like. The compositions can also include one or more permeation enhancers including, for example, vitamin E, L-Arginine, L-Valine, dimethylsulfoxide (DMSO), dimethyl formamide (DMF), N,N-dimethyl-acetamide (DMA), decylmethylsulfoxide (C₁₀MSO), polyethylene glycol monolaurate (PEGML), glyceral monolaurate, lecithin, 1-substituted azacycloheptan-2-ones, particularly 1-N-dodecylcyclazacylcoheptan-2-ones (available under the trademark Azone from Nelson Research & Development Co., (Irvine, Calif.), alcohols and the like.

### Transdermal Delivery

Sequence modified CGRP may also be administered to a patient via transdermal delivery devices, patches, electrophoretic devices, bandages and the like. Such transdermal patches may be used to provide continuous or discontinuous infusion of sequence modified CGRP in controlled amounts. The construction and use of transdermal patches for the delivery of pharmaceutical agents is well known in the art. See, for example, U.S. Pat. No. 5,023,252, the disclosure of which is herein incorporated by reference. Such patches may be constructed for continuous, pulsatile, or on-demand delivery of sequence modified CGRP. For example, a dose of sequence modified CGRP or a pharmaceutical composition thereof may be combined with skin penetration enhancers including, but not limited to, oleic acid, oleyl alcohol, long chain fatty acids, propylene glycol, polyethylene glycol, isopropanol, ethoxydiglycol, sodium xylene sulfonate, ethanol, N-methylpyrrolidone, laurocapram, alkanecarboxylic acids, dimethylsulfoxide, polar lipids, N-methyl-2-pyrrolidone, and the like, which increase the permeability of the skin to the dose of sequence modified CGRP and permit the dose of sequence modified CGRP to penetrate through the skin and into the bloodstream. Sequence modified CGRP or a acceptable composition thereof may be combined one or more agents including, but not limited to, alcohols, moisturizers, humectants, oils, emulsifiers, thickeners, thinners, surface active agents, fragrances, preservatives, antioxidants, vitamins, or minerals. Sequence Modified CGRP or a pharmaceutical composition thereof may also be combined with a polymeric substance including, but not limited to, ethylcellulose, hydroxypropyl cellulose, ethylene/vinylacetate, polyvinyl pyrrolidone, and the like, to provide the composition in gel form, which may be dissolved in solvent such as methylene chloride, evaporated to the desired viscosity, and then applied to backing material to provide a patch. The backing can be any of the conventional materials such as polyethylene, ethyl-vinyl acetate copolymer, polyurethane and the like.

### Transmucosal Delivery

Sequence modified CGRP may also be administered transmucosally, i.e., to and across a mucosal surface, for example, for the treatment of angina. Transmucosal administration of a source of sequence modified CGRP or a pharmaceutical composition thereof can be accomplished generally by contacting an intact mucous membrane with a source of sequence modified CGRP or a pharmaceutical composition thereof, and maintaining the source in contact with the mucous membrane for a sufficient time period to induce the desired therapeutic effect. Preferably, sequence modified CGRP or a pharmaceutical composition thereof is administered to the oral or nasal mucosa such as the buccal mucosa, the sublingual mucosa, the sinuidal mucosa, the gum, or the inner lip. Particularly, the source of sequence modified CGRP is any preparation usable in oral, nasal, sinuidal, rectal, or vaginal cavities that can be formulated using conventional techniques well known in the art. For example, the preparation can be a buccal tablet, a sublingual tablet, a spray, and the like that dissolves or disintegrates, delivering drug into the mouth of the patient. A spray or drops can also be used to deliver the sequence modified CGRP or a pharmaceutical composition thereof to nasal or sinuidal cavities. The preparation may or may not deliver the drug in a sustained fashion. Examples for manufacturing such preparations are disclosed, for example, in U.S. Pat. No. 4,764,378, which is specifically incorporated herein by reference. The preparation can also be a syrup that adheres to the mucous membrane. Suitable mucoadhesives include those well known in the art such as polyacrylic acids, preferably having the molecular weight between from about 450,000 to about 4,000,000 (e.g., Carbopol™ 934P); sodium carboxymethylcellulose (NaCMC), hydroxypropylmethylcellulose (HPMC) (e.g., Methocel™ K100, and hydroxypropylcellulose).

The transmucosal preparation can also be in the form of a bandage, patch, and the like that contains the drug and adheres to a mucosal surface. A mucoadhesive preparation is one that upon contact with intact mucous membrane adheres to the mucous membrane for a sufficient time period to induce the desired therapeutic effect. Suitable transmucosal patches are described for example in PCT Publication WO 93/23011, which is specifically incorporated herein by reference. A suitable patch may comprise a backing which can be any flexible film that prevents bulk fluid flow and provides a barrier for to loss of the drug from the patch. The backing can be any conventional material such as polyethylene, ethyl-vinyl acetate copolymer, polyurethane and the like. In a patch involving a matrix which is not itself a mucoadhesive, the drug-containing matrix can be coupled with a mucoadhesive component (such as a mucoadhesive described above) in order that the patch may be retained on the mucosal surface. Suitable configurations include a patch or device wherein the matrix has a smaller periphery than the backing layer such that a portion of the backing layer extends outward from the periphery of the matrix. A mucoadhesive layer covers the outward extending portion of the backing layer such that the underside of the backing layer carries a layer of mucoadhesive around its periphery. The backing and the peripheral ring of mucoadhesive taken together form a reservoir which contains a drug-containing matrix (e.g. a tablet, gel or powder). It may be desirable to incorporate a barrier element between the matrix and the mucoadhesive in order to isolate the mucoadhesive from the matrix. The barrier element is preferably substantially impermeable to water and to the mucosal fluids that will be present at intended site of adhesion. A patch or device having such barrier element can be hydrated only through a surface that is in contact with the mucosa, and it is not hydrated via the reservoir. Such patches can be prepared by general methods well known to those skilled in the art. The preparation can also be a gel or film comprising a mucoadhesive matrix as described for example in PCT Publication WO 96/30013, which is specifically incorporated herein by reference.

Suppositories for administration of the compounds and/or compositions of the invention can be prepared by mixing the drug with a suitable nonirritating excipient such as cocoa butter and polyethylene glycols which are solid at room temperature but liquid at vaginal temperature, such that they will melt in the vagina and release the drug.

### Implantable Pumps

In another embodiment, sequence modified CGRP can be suitably administered using an implantable pump, which is particularly applicable for outpatient treatment. For example, a constant rate pump may be used to provide a constant, unchanging delivery of sequence modified CGRP over a period of time. Alternatively, a programmable, variable rate pump may be used if changes to the infusion rate are desired. Constant rate and programmable pumps are well know in the art and need not be described further.

Sequence modified CGRP may also be released or delivered from an implantable osmotic mini-pump such as that described in U.S. Pat. Nos. 5,728,396, 5,985,305, 6,358,247, and 6,544,252, the disclosures of which are specifically incorporated herein in their entirety. The release rate from an osmotic mini-pump may be modulated with a microporous, fast-response gel disposed in the release orifice for controlled release or targeted delivery of sequence modified CGRP. Osmotic pumps are preferred in that they are much smaller than the constant rate and programmable pumps.

In one embodiment, the osmotic pump comprises a miniature drug-dispensing system that operates like a miniature syringe and releases minute quantities of concentrated sequence modified CGRP formulations in a continuous, consistent flow over months or years. The system is implanted under the skin and can be as small as 4 mm OD×44 mm in length or smaller. Such a system is sold under the trademark DUROS.™ (ALZA Corporation). In brief, such an osmotic delivery system comprises a capsule having an interior that contains the sequence modified CGRP and ah osmotic agent, a semi-permeable body that permits liquid to permeate through the body to the osmotic agent, and a piston located within the interior of the capsule that defines a movable seal within the interior that separated the osmotic agent from the sequence modified CGRP.

It is contemplated that all of the above compositions and methods of delivery can be used with the retro-inverso form or pharmaceutically acceptable salt thereof in the same manner as described for the sequence modified CGRP compounds.

### Kits

The present invention also provides pharmaceutical kits comprising one or more sequence modified CGRP including corresponding retro-inverso forms compositions of this invention. Such kits can also include additional drugs or therapeutics (e.g., antiproliferative or anti-clotting agents, or other compounds used to treat cardiovascular diseases and the like) for co-use with sequence modified CGRP for treatment or prevention of HF and/or for improving renal failure. In this embodiment, the sequence modified CGRP and the drug can be formulated in admixture in one container, or can be contained in separate containers for simultaneous or separate administration. The kit can further comprise a device(s) for administering the compounds and/or compositions, and written instructions in a form prescribed by a governmental agency regulating the manufacture, use or sale of pharmaceuticals or biological products, which instructions can also reflect approval by the agency of manufacture, use or sale for human administration.

The present invention also provides pharmaceutical kits comprising one or more containers filled with one or more of the ingredients of the pharmaceutical compounds and/or compositions of the present invention, including, one or more of hydrophobic-sequence modified CGRP and/or other vasoactive. One embodiment would include a container having at least two distinct chambers, wherein one chamber would contain hydrophobic-sequence modified CGRP and another chamber would contain the delivery vehicle, such that as the hydrophobic-sequence modified CGRP and delivery vehicle are dispensed they are also mixed prior to application. Such kits can also include, for example, other compounds and/or compositions (e.g., permeation enhancers, lubricants), a device(s) for administering the compounds and/or compositions, and written instructions in a form prescribed by a governmental agency regulating the manufacture, use or sale of pharmaceuticals or biological products, which instructions can also reflects approval by the agency of manufacture, use or sale for human administration.

### Sequence Modified CGRP Administration

Generally, the dosage required to provide an effective amount of sequence modified CGRP or a pharmaceutically acceptable salt thereof including corresponding retro-inverso forms, is within the ranges disclosed herein and can be adjusted by one of ordinary skill in the art. The dosage will vary depending on the clinical condition of the individual patient (especially the side effects of treatment with sequence modified CGRP alone or in combination with other therapeutics), the age, health, physical condition, sex, diet and medical condition of the patient, the severity (i.e., stage) of the condition, the route of administration, the site of delivery of sequence modified CGRP, the type of drug delivery system that is used, whether sequence modified CGRP is administered as part of a drug combination, the scheduling of administration, and other factors known to practitioners. Thus, while individual needs may vary, determination of optimal ranges for effective amounts of sequence modified CGRP (alone or in combination with other drugs) within the ranged disclosed herein is within the expertise of those skilled in the art. Accordingly, “effective amounts” of each component for purposes herein, are determined by such considerations and are amounts that improve one or more hemodynamic functions and/or ameliorate on or more deleterious conditions in HF patients and/or improve the quality of life in HF patients and/or improve renal function.

If necessary, sequence modified CGRP can be administered according to the methods of this invention either alone or in combination with at least one other agent including, but not limited to, anti-proliferative agents, anti-clotting agents, vasodilators, diuretics, beta-blockers, calcium ion channel blockers, blood thinners, cardiotonics, ACE inhibitors, anti-inflammatories, anti-platelet drugs, thrombolytic agents, antioxidants, and/or gene therapeutics. When used in combination with other agents, sequence modified CGRP and the agent can be administered separately (either simultaneously or separately in any order) or in admixture. In one embodiment, when sequence modified CGRP and at least one other agent are administered as separate components, they are administered to the patient at about the same time. “About the same time” means that within about thirty minutes of administering one compound (e.g., sequence modified CGRP) to the patient, the other compound (e.g., an anti-proliferative or anti-clotting agent) is administered to the patient. “About the same time” also includes concomitant or simultaneous administration of the compounds.

The route of administration of the sequence modified CGRP will vary depending upon the contemplated application. For specialized vascular beds, such as the coronary, carotid, or renal arteries, the sequence modified CGRP preferably should be administered by selected intraarterial application. The dose of sequence modified CGRP preferably should be between about 0.25 and 1 nmol administered as a bolus dose or as an infusion of 2-10 pmol/kg/minute. The sequence modified CGRP may be in either free or liposomal form. To get the maximum benefit, the sequence modified CGRP should be administered via a catheter directly into the artery of interest. The dose preferably should be pre-loaded into a catheter suitable for insertion into the target artery for local administration of the dose. However, in order to make the sequence modified CGRP as economical as possible, variable doses also may be used, preferably via a second channel in the catheter. In addition, some patients may benefit from local infusion of sequence modified CGRP after administration of the initial bolus dose, preferably via the same catheter. In certain patients, sequence modified CGRP can be delivered directly onto the arterial wall (i.e., at the site of narrowing or spasm) through a “leaky catheter” after balloon inflation or by a coronary stent impregnated with sequence modified CGRP.

In a preferred embodiment, the sequence modified CGRP is introduced during angioplasty, through the angioplasty catheter, itself. Most of the available balloon catheters have two passageways or lumens. A first passageway is used to inflate and deflate the balloon with a hydraulic system. A second passageway is used to pass the guidewire through the catheter. Typically, this second passageway is large enough to maintain a channel around the guidewire to permit monitoring of the vascular pressure at the distal tip of the catheter or to permit monitoring of the vascular anatomy by radiographic dye injection. The channel also can be used to transport drugs through the catheter for application to the artery distal to the catheter. A preferred embodiment involves the administration of sequence modified CGRP through an angioplasty catheter, preferably the CORFLO™ perfusion catheter (available from Leocor, Inc., Houston, Tex.).

Where the use of a catheter is contraindicated, or where the use of a catheter would involve a significant delay for some technical reason, e.g., more than 30 minutes or so, the sequence modified CGRP may be administered as an intravenous infusion at 0.1 to 0.5 nmol/kg/hr.

Where the goal is to prevent the reocclusion of arteries after angioplasty (e.g., balloon angioplasty, mechanical dilatation, application of rotorouter, etc.) or vascular grafts in any vascular bed (e.g., coronary, renal, carotid, femoral, etc.), a similar dose should be administered. Preferably, 0.25 to 1 nmol of free or liposomal sequence modified CGRP should be pre-loaded into a catheter and administered directly to the target artery as a bolus dose. Alternately, 2 to 10 pmol/kg/minute of the sequence modified CGRP may be infused locally into the target artery through the catheter. In order to make the product as economical as possible, the catheter should be adapted for infusion of a second dose or infusions locally to the target artery. In some cases, it may be beneficial for the local infusion of sequence modified CGRP to last for a longer period of time after the initial bolus dose. In addition, following an angioplasty procedure, a low dose intravenous infusion of 50-200 pmol/kg/hr of sequence modified CGRP is recommended, provided that the blood pressure is satisfactorily maintained.

Where the sequence modified CGRP is used to prevent reocclusion of vessels following arterial or venous grafts, the sequence modified CGRP preferably should be infused into a peripheral vein at a dose of about 50-200 pmol/kg/hr for several hours, preferably for at least about 8-24 hours, as in the case of administration of intravenous heparin or nitrates. For this particular use, local infusion via a catheter may not be required.

Selective arterial infusion or injection of a localized bolus dose of sequence modified CGRP (e.g., intracoronary, or intracarotid) causes a localized vasodilation of the arterial bed concerned. However, intravenous infusion of sequence modified CGRP causes a preferential increase of blood flow to the heart, kidney, brain, and skin in the upper half of the body, respectively. Where clotting is a concern, the beneficial effects of sequence modified CGRP may be enhanced by co-administering the sequence modified CGRP with an anti-platelet drug, such as prostaglandin E₁, aspirin, ticlopidine, dipiridamol, an aspirin-like compound, or a thrombolytic agent (clot buster) such as recombinant tissue plasminogen activator (rTPA) or streptokinase. sequence modified CGRP, itself, does not have an anti-platelet effect.

Injection of a bolus dose (or an infusion) of sequence modified CGRP into the coronary arteries of humans to relieve ischemia associated with coronary artery narrowing (permanent) or spasms (temporary narrowing) has relieved cardiac pain and reversed electrocardiographic changes associated with ischemia. Preliminary studies with miniature doplers confirmed a significant increase of blood flow in the coronary arteries after infusion of sequence modified CGRP.

### Parenteral Administration

According to one embodiment, this invention provides a method of treating a patient comprising administering sequence modified CGRP or a pharmaceutical composition thereof to the patient at a rate between about 50 and 500 ng/min for a time between 30 minutes and 8 hours per day for as many days as needed to provide symptomatic relief, prevent exacerbation of symptoms, and/or prevent and/or delay progression of the disease state in the patient. For example, sequence modified CGRP may be continuously or intermittently administered for a period of time between about 24 and 48 hours, or as a bolus dose. If sequence modified CGRP is administered two or more times intermittently each day, lower doses, e.g., 0.8 to 10 ng/min can be administered.

According to another embodiment, a method of treating a patient is provided, whereby about 0.8 to about 30 ng/kg/min is administered for a time as needed, such as between 30 minutes and 8 hours per day, for a period of time between about 24 and about 48 hours, or as a bolus dose, continuously or intermittently, as needed to provide symptomatic relief, prevent exacerbation of symptoms, and/or prevent and/or delay progression of the disease state in a patient. In preferred embodiments, about 29 ng/kg/min is administered. In other preferred embodiments, about 8 ng/kg/min is administered.

Treatment is continued as needed to provide symptomatic relief, prevent exacerbation of symptoms, and/or prevent and/or delay progression of the disease state in the patient, or until it is no longer well tolerated by the patient, or until a physician terminates treatment. For example, a physician may monitor one or more symptoms of HF, renal blood flow, glomerular filtration rates, and/or serum levels of urea and creatinine in a patient being treated with sequence modified CGRP according to this invention and, upon observing attenuation of one or more symptoms of HF for a period of time, conclude that the patient can sustain the positive effects of the above-described treatment without further administration of sequence modified CGRP for a period of time. If necessary, the patient may then return at a later point in time for additional treatment as needed.

According to another embodiment, this invention provides a method of treating a patient comprising administering sequence modified CGRP to the patient at a rate between about 500 and 600 ng/min for period between about 8 hours per day for at least three consecutive days or several times per week as needed to provide symptomatic relief, prevent exacerbation of symptoms, and/or prevent and/or delay progression of the disease state of heart failure in the patient. This treatment may be provided as outpatient therapy to prevent exacerbation of symptoms and to enhance the quality of life in the patient.

As used herein, “day” means a 24-hour period. Thus, for example, “for at least three consecutive days” means for at least a 72-hour period. During or after the treatment, a physician may monitor one or more symptoms of HF, renal blood flow, glomerular filtration rates, and/or serum levels of urea or creatinine in the patient and, upon observing an improvement in one or more of the parameters for a period of time, conclude that the patient can sustain the positive effects of the treatment without further administration of sequence modified CGRP for a period of time.

According to another embodiment, this invention provides a method of treating a patient comprising administering sequence modified CGRP to the patient at a rate between about 50 and 400 ng/min over a period of up to 8 hours per day for each day of hospitalization of the patient or as needed. In certain cases the patient may require higher doses, for example up to 2 μg/min over the same time period.

Once treatment with sequence modified CGRP according to any of the methods of this invention has achieved the desired results, e.g., symptomatic relief, prevent exacerbation of symptoms, and/or prevent and/or delay progression of disease states, the patient can then receive maintenance therapy if desired. For example, a lower dose of sequence modified CGRP, e.g., less than 10 ng/min, can be administered to the patient for maintenance therapy by any suitable route including, but not limited to, injection, intravenous administration, etc. In one embodiment, the delivery regime can be designed to deliver between sequence modified CGRP at a rate between about 0.8 to 10 ng/min for a desired period of time, such as over a period of 3, 6 or 9 months.

Because sequence modified CGRP therapy according to any of the methods of this invention prevents further damage from ischemic injury and promotes the healing process, it can also be used to delay or preclude further exacerbation of a heart condition into a more serious and progressive diseases such as HF. Thus, each of the above-described methods may also be used as a prophylactic treatment to prevent or slow the progression of early stages of HF to more advanced stages. That is, once treatment with sequence modified CGRP according to any of the methods of this invention has achieved the desired results, the patient can optionally receive maintenance therapy thereafter. For example, one embodiment of this invention for providing maintenance therapy to a patient with a heart condition comprises providing a lower dose of sequence modified CGRP, e.g., less than 10 ng/min, to the patient for maintenance therapy by any suitable route including, but not limited to, injection, intravenous administration, controlled release administration, etc. In one embodiment, the delivery system can be designed to deliver between sequence modified CGRP at a rate between about 0.8 to 10 ng/min for a desired period of time, such as over a period of 3, 6 or 9 months. In an alternative embodiment, the patient can receive long-term, low dose, maintenance administration of sequence modified CGRP from a controlled release formulation.

This invention further provides methods of treating a patient comprising administering sequence modified CGRP to the patient such that circulating plasma levels of sequence modified CGRP are sufficient to maintain hemodynamic stability, thereby preventing or delaying exacerbation of symptoms. For example, in prior clinical studies using Stage C and DHF patients, effective circulating plasma levels of CGRP were administered by intravenous infusions ranging between 157±26 pg/mL to 186±127 pg/mL (Anand; et al., 1991 and Shekhar, et al., 1991, supra). However, these doses could only be administered intravenously for about 12-24 hours before unwanted side effects set in and the IV administration had to be discontinued. In contrast, the methods of the present invention administer sequence modified CGRP by controlled release systems or compositions that maintain circulating plasma levels of sequence modified CGRP between about 11±5 pg/mL and 186±127 pg/mL for a length of time that is within the capabilities of the particular controlled release delivery system or composition.

The amount of a given of hydrophobic-sequence modified CGRP and/or other vasoactive agents of the present invention which will be effective in the treatment of a particular dysfunction or condition will depend on the nature of the dysfunction or condition, and can be determined by standard clinical techniques, including reference to Goodman and Gilman, “The Pharmacological Basis of Therapeutics” (9th Edition, 1995); “The Physician's Desk Reference” (49th Ed.); “Medical Economics” (1995); “Drug Facts and Comparisons” (1993); and “The Merck Index (12^{th} Ed.)”, Merck & Co., Inc. (1996), the disclosures of each of which are incorporated herein by reference in their entirety. The precise dose to be used in the formulation will also depend on the route of administration, and the seriousness of the dysfunction or disorder, and should be decided by the physician and the patient's circumstances.

It is contemplated that the usual dose of hydrophobic-sequence modified CGRP and/or other vasoactive agents administered to a patient is about 1.0 μg/dose to about 100.0 μg/dose, preferably about 5.0 μg/dose to about 50 μg/dose, more preferably about 10.0 μg/dose. Effective doses can be extrapolated from dose-response curves derived from in vitro or animal model test systems and are in the same ranges or less than those described for other commercially available compounds in, for example, the Physician's Desk Reference (49th Ed.).

### Methods of Treatment Using Sequence Modified CGRP

Administering sequence modified CGRP including corresponding retro-inverso forms according to the methods of this invention provides a safer and more effective treatment of acute cardiac ischemia and heart failure compared to current treatments for HF. Given the advantages in cardioprotection, myocardial tissue salvage, cardiac hemodynamic improvement, and renal function provided by sequence modified CGRP, the methods of this invention have the potential to be powerful frontline weapons in the arsenal of emergency room doctors who are the first to treat patients suffering from myocardial infarction (MI) upon entry into the health care system, and/or an interventional cardiologist who is working to re-establishing blood flow to an ischemic heart using angioplasty or stenting procedures, and/or a cardiologist who is treating mid- to end-stage heart failure patients to provide increased quality of life to terminal patients.

The compounds and methods described herein including the controlled release compositions can further be used for maintenance therapies, preferably using lower doses or dosing rates of sequence modified CGRP, after the initial therapy is completed.

### Heart Failure

Heart failure (HF) is a complex clinical syndrome that can result from any structural or functional cardiac disorder that impairs the ability of the ventricle to fill with or eject blood, and the heart works less efficiently than it should. Heart failure is characterized by specific symptoms (e.g., dyspnea and fatigue) which may limit exercise tolerance and signs. (e.g., fluid retention) which may lead to pulmonary congestion and peripheral edema. Both abnormalities can impair the functional capacity and quality of life of affected individuals, but they may not necessarily dominate the clinical picture at the same time. Because not all patients have volume overload at the time of initial or subsequent evaluation, the term “heart failure” is preferred over the older term “congestive heart failure”.

The clinical syndrome of heart failure may result from disorders of the pericardium, myocardium, endocardium, or great vessel. For example, common causes of heart failure include: narrowing of the arteries supplying blood to the heart muscle (coronary heart disease); prior heart attack (myocardial infarction) resulting in scar tissue large enough to interfere with normal function of the heart; high blood pressure; heart valve disease due to past rheumatic fever or an abnormality present at birth; primary disease of the heart muscle itself (cardiomyopathy); defects in the heart present at birth (congenital heart disease) and infection of the heart valves and/or muscle itself (endocarditis and/or myocarditis or pericarditis). The majority of patients with heart failure have symptoms due to an impairment of left ventricular function. Each of these disease processes can lead to heart failure by reducing the strength and efficiency of the heart muscle contraction, by limiting the ability of the heart's pumping chambers to fill with blood due to mechanical problems or impaired diastolic relaxation, or by filling the chambers with too much blood.

Renal blood flow is also an important factor in the development of the clinical syndrome of heart failure. It is a determinant of some important neurohormonal responses and of salt and water retention. Renal blood flow is reduced in patients with HF, and many patients with HF will also eventually develop renal failure.

There are four stages of heart failure recognized by the American College of Cardiology Guidelines for the Evaluation and Management of Chronic Heart Failure in the Adult. Stage A refers to patients who are at high risk for developing heart failure but have no identified structural or functional abnormalities of the heart and have never shown signs or symptoms of heart failure. If needed, Stage A patients are prescribed ACE inhibitors to lower blood pressure and reduce the heart's workload. Stage B refers to patients who have developed structural heart disease strongly associated with the development of heart failure but have never shown signs or symptoms of heart failure. Stage B patients are typically prescribed ACE inhibitors and beta-blockers that decrease myocardial oxygen demand and thereby ischemia, and reduce heart rate and cardiac work. Stage C refers to current or prior symptoms of heart failure associated with underlying structural disease. Management of HF at Stage C can involve a triple or quadruple drug therapy that includes ACE inhibitors, beta-blockers, diuretics, and Digitalis. Stage D refers to patients with advanced structural heart disease and marked symptoms of heart failure at rest despite maximal medical therapy, requiring specialized intervention. Since HF is a terminal condition, mid and end-stage HF (Stages C and D, respectively) treatment focuses on alleviating symptoms and increasing the patient's quality of life such that they can continue to live a relatively active lifestyle. Successful management of the progression of heart failure and effective treatments to relieve heart failure symptoms are determined by monitoring increases in the heart's ejection fraction, decreases in dyspnea, and changes in the frequency and/or severity of heart failure symptoms. However, while current end-stage drug therapies such as Dobutamine or Milrinone increase the patient's quality of life, they also have been shown to increase mortality.

It is estimated that about four million people in the United States suffer from various degrees of heart failure. Although heart failure is a chronic condition, the disease often requires acute hospital care. Patients are commonly admitted for acute pulmonary congestion accompanied by serious or severe shortness of breath. Acute care for HF accounts for the use of more hospital days than any other cardiac diagnosis, and consumes in excess of seven and one-half billion dollars in the United States annually.

Current research into the treatment of chronic heart failure is focused on providing cardioprotection, myocardial tissue salvage by minimizing or reducing infarction size, and preventing reperfusion injury. Many current drug therapies for treating heart failure address specific clinical aspects associated with myocardial infarction, such as anti-platelet/fibrinolytic, anti-inflammatory, and antioxidant activities. Such drugs include ACE inhibitors to prevent blood vessel constriction and to increase blood flow to the body, diuretics to remove excess fluid, beta blockers to reduce heart work load, calcium channel blockers to increase the blood flow through the heart and prevent vessel constriction, blood thinners to prevent blood clots, and cardiotonics to strengthen the heart's ability to pump blood. Only a few companies to date are developing new drugs that address tissue salvage, however the effectiveness of these drugs remains to be established in the clinic. As with all drugs, these agents must be taken in doses sufficient to ensure their effectiveness. Problematically, however, over-treatment can lead to hypotension, renal impairment, hyponatremia, hypokalemia, worsening heart failure, impaired mental functioning, and other adverse conditions. Surgical treatments include angioplasty, coronary arty by-pass grafts, valve replacement, pacemakers, internal defibrillators, left ventricular assist devices, and heart transplants.

Heart failure is the number one diagnosis for hospital admissions in patients over the age of 65. More than $38.1 billion has been spent annually since 1991 on inpatient and outpatient costs and greater than $500 million on drugs to treat HF. The disorder is the underlying reason for 12 to 15 million office visits each year and 1.7 to 2.6, million hospital admissions each year. Because of the hospitalization costs required to treat a heart failure patient, the current trend is to get HF patients into outpatient care as soon as possible, often within the 48 hours of hospital admission. Specialized outpatient clinics are now available for heart failure patients. The patients typically attend the clinic between one and four times per week to receive intravenous infusions of a prescribed heart failure therapy until hemodynamic symptoms improve.

When administered, CGRP has pronounced cardiovascular benefits, including vasodilation, ischemic cardioprotection, reduction in infarction size due to heart attack, inhibition of platelet aggregation and smooth muscle cell proliferation that can potentially reduce the incidence of restenosis, increased renal function, and overall increased efficiency of cardiovascular functions. As a result of providing cardioprotection, minimizing reperfusion injury, and reducing infarction size, CGRP also promotes myocardial tissue salvage. CGRP also plays a role in regulating inotropy, chronotropy, microvascular permeability, vascular tone, and angiogenesis. CGRP also has significant advantages over conventional drug treatments. First, CGRP does not produce the potentially dangerous side effects, toxicity, and tolerance associated with conventional cardiovascular drugs such as Nitroglycerin, Dobutamine, and Natrecor. In fact, CGRP has been reported to down-regulate immune response via inhibition of cytokine release and has been safely administered to immuno-suppressed subjects without-the induction of sensitivity. Second, because CGRP has multiple hemodynamic benefits, it can potentially reduce or eliminate the need for drug cocktails to maintain specific hemodynamic functions. Third, the biochemical activity of CGRP is mediated through specific receptor binding sites concentrated in the heart, kidneys, and genitalia, and is known, to act on two specific CGRP receptor subtypes located on the surface of the endothelial and smooth muscle cells, respectively. Accordingly, CGRP exhibits virtually no side effects or tolerance when administered systemically.

Studies have demonstrated that acute administration of CGRP can result in increased cardiac performance and reduced systemic resistance in a number of clinical scenarios. For example, Anand, et al. J. Am. Coll. Cardiol., 17:208-217 (1991)) reported that short-term IV infusions (10 or 20 minutes) of CGRP at rates of 0.8, 3.2, or 16 ng/kg/min (i.e., 56, 224, or 1120 ng/min based on a 70 kg subject) produced beneficial hemodynamic effects such as decreased systemic vascular resistance and increase in cardiac output, with no tachycardia observed. The study concluded that at lower doses CGRP behaves as a pure arteriolar vasodilator, where as at the higher dose CGRP acts a mixed vasodilator. Stephenson, et al. (Int. J. Cardiol., 37:407-414 (1992)) reported administration of CGRP at a rate of 600 ng/min by either a 48-hour continuous IV-infusion or 2-8 hour infusions for two consecutive days. In the continuous infusion therapy, infusion was discontinued after 28 hours in 3 out of the 6 patients due to nausea, diarrhea, and/or severe facial flushing. On the other hand, the pulsed therapy was well tolerated and was observed to improve hemodynamic functions such as left ventricular function. However, unfavorable side effects of tachycardia and neurohumoral response were also observed with the pulsed therapy. Sekhar, et al. (Am. J. Cardiol. 67:732-736 (1991) reported administration of CGRP at a rate of 8 ng/kg/min (i.e., 560 ng/min based on a 70 kg subject) by IV infusion for 8 hours. This therapy was observed to have beneficial hemodynamic effects such as decreased pulmonary and systemic arterial pressure, decreased vascular resistance and increased cardiac output. It was also observed that renal blood flow and glomerular filtration were increased during treatment. However, the hemodynamic effects were lost within 30 minutes of stopping CGRP infusion.

One object of this invention provides improved methods for treating heart failure (HF) by administering sequence modified CGRP to a patient having HF in a manner effective to treat or prevent HF. The “patient” can be any living organism, including a warm-blooded mammal such as a human. The treatment according to any of the methods of this invention can be administered on an inpatient such as a hospital or emergency room, or in an outpatient setting such as a hospice or home health care setting or administration by emergency care personnel to a patient having a myocardial infarction. This invention further provides methods of improving hemodynamic functions in a patient with HF by administering controlled release, sequence modified CGRP to the patient in either an inpatient or outpatient setting.

The present invention provides a method for the treatment or prevention of heart failure by administering one or more doses of a sequence modified CGRP formulation in a manner that will treat the conditions underlying HF while minimizing or attenuating deleterious effects commonly associated with CGRP such as nausea, diarrhea, severe facial flushing and intermittent tachycardia. More specifically, this invention provides improved dosing regimes using sequence modified CGRP for patients suffering from or at risk for HF, and a method of treating-HF or delaying the progression of HF into more advanced stages by providing lower dose and longer term administration using sequence modified CGRP. This invention also provides a method of improving the quality of life in a patient with HF.

When administered in controlled dosages, sequence modified CGRP has pronounced cardiovascular benefits, including vasodilation, ischemic cardioprotection, reduction in infarction size due to heart attack, inhibition of platelet aggregation and smooth muscle cell proliferation to potentially reduce the incidence of restenosis, increased renal function, and overall increased efficiency of cardiovascular functions. sequence modified CGRP also plays a role in regulating inotropy, chronotropy, microvascular permeability, vascular tone, and angiogenesis.

In general, there are four goals in treating HF patients: (1) treating the symptoms, (2) slowing the progression of cardiac dysfunction, (3) decreasing length of hospital stay, and (4) increasing the time between hospitalization, all while minimizing health care costs. It is believed that the methods for the treatment or prophylaxis of HF according to this invention will achieve one or more of these goals.

A further aspect of this invention provides a method of treating HF by administering sequence modified CGRP according to any of the methods disclosed herein to augment current HF therapies. sequence modified CGRP can be administered according to any of the dosing regimes of this invention together with one or more addition drugs for HF, wherein sequence modified CGRP and the additional drug(s) can be administered together, separately and simultaneously, or separately in any order.

This invention provides prophylactic methods of preventing HF in a patient at risk of HF or slowing the progression or symptoms of HF in a patient suffering from HF. For example, another aspect of this invention provides a method of preventing or reducing the risk of occurrence of myocardial infarction in a patient, comprising administering to a human at risk of having a myocardial infarction a controlled release sequence modified CGRP formulation in an amount effective to prevent or reduce the risk of myocardial infarction.

### Hemorrhagic Shock

Hemorrhagic shock is shock associated with the sudden and rapid loss of significant amounts of blood. Severe traumatic injuries often cause such blood losses. This results in inadequate perfusion to meet the metabolic demands of cellular function. Death occurs within a relatively short time unless transfusion quickly restores normal blood volume. Hemorrhagic shock often accompanies secondary shock. Hemorrhagic shock is characterized by hypotension, tachycardia, oliguria, and by pale, cold, and clammy skin.

### Myocardial Infarction (MI)

It is known that a patient that has suffered a myocardial infarction (MI) will likely suffer another MI in the future. Thus, a patient having an MI can be treated with an initial dose of sequence modified CGRP according to any of the methods of this invention until one or more symptoms of HF has diminished, and subsequently can be put on a sequence modified CGRP maintenance dosing regime. The maintenance regime can also be given to a post-MI patient that was initially treated for MI by means other than sequence modified CGRP, and can also be used for HF patients that have not yet suffered an MI as a means to slow the progression of HF into the more advanced stages or to prevent or reduce the risk of MI in patients with advanced HF.

In the treatment of acute MI, physicians take aggressive action to restore blood flow to the heart to minimize permanent ischemic damage. These treatments take the form of vasodilators (nitroglycerin) and antithrombolytics. (streptokinase, tPA), and platelet aggregation inhibitors (gpIIb/IIIa) in the attempt to dilate the coronary arteries and dissolve the thrombus, and inhibit platelet aggregation. If treatment is successful in restoring blood flow, the patient may be sent to recover in the CCU or go to the catheterization lab for an angioplasty or stenting procedure to open any remaining occlusions. However, the ischemic event itself causes generation of free radicals, and this process is potentiated when the vessels are re-opened and blood flow restored, which results in further tissue damage. In this setting, sequence modified CGRP therapy administered alone or in conjunction with other therapeutic interventions according to any of the methods of this invention, particularly the infusion methods, would augment the current therapies such as antithrombolytics by elevating the therapeutic benefits of these drugs (see International Pub No. WO 2005/070445 A2, the disclosure of which is incorporated herein by reference in its entirety). The cardioprotective benefits of sequence modified CGRP when infused at the initial stages of evaluation and treatment would provide levels of sequence modified CGRP suitable to minimize reperfusion injury when interventional therapy is initiated, and thus maximize positive acute and long-term recovery outcomes.

Accordingly, this invention further provides a method of counteracting ischemia due to myocardial infarction in a patient, comprising delivering to the patient an amount of sequence modified CGRP effective to provide cardioprotection, reduction in infarction size, reduction in reperfusion injury, symptomatic relief, and/or prevent exacerbation of symptoms, wherein the sequence modified CGRP is delivered to the patient as a controlled release composition.

### Pulmonary Hypertension

Pulmonary hypertension is an increase in blood pressure in the lung vasculature (pulmonary artery, pulmonary vein, pulmonary capillaries), leading to shortness of breath, dizziness, fainting, and other symptoms, all of which are exacerbated by exertion. Pulmonary hypertension can be a severe disease with a markedly decreased exercise tolerance and heart failure. Pulmonary hypertension can be one of five different types: arterial, venous, hypoxic, thromboembolic or miscellaneous.

### Vasospasms

A number of vasoconstrictor peptides produced by the body induce vasospasms, particularly when blood supply to a region is reduced for some reason. Vasospasms are common when a patient is afflicted with an obstructed artery, such as the coronary artery, and during ischemia associated with many organs, such as the heart, brain, and kidney. Vasospasms are particularly common during and after medical procedures that reduce the blood flow in the vicinity of the procedure. Examples of such procedures are angioplasty and the implantation of vascular grafts. Vasospasms also are a contributing factor to the reocclusion of arteries after angioplasty, stent insertion, or other reconstructive arterial, or trauma surgery.

Angioplasty is a procedure for dilating an obstructed artery. One common type of angioplasty is known as percutaneous transluminal coronary angioplasty (“PTCA”). PTCA is performed using a “balloon” catheter, or a PTCA catheter. A balloon catheter consists, very basically, of an inflatable balloon and a means for guiding the balloon to the target occlusion and for inflating the balloon to dilate the artery at the point of the occlusion. Preferably, the catheter also permits simultaneous monitoring of aortic pressure and/or simultaneous dye injection to clarify the vascular anatomy.

During angioplasty, the blood flow through the target artery is greatly reduced, resulting in angina. As a result, it often is necessary to infuse drugs or oxygenated blood distal to the stenosis in order to maintain adequate physiological function of the target organ. The drugs that are commonly used to dilate the artery are substances that do not naturally occur in the human body. These foreign substances have the potential for toxicity and for inducing an allergic reaction. Since the patient already is in a stressed condition due to the angioplasty procedure, itself, such reactions are undesirable. A more natural method for dilating arteries to counteract vasospasms, which does not create such a risk of toxicity and allergic reaction, would be highly desirable.

### Percutaneous Transluminal Coronary Angioplasty (PTCA) and Stenting

If antithrombolytic therapy is ineffectual in the emergency room, or if it is determined that elective PTCA intervention is required to restore blood flow, sequence modified CGRP infusion therapy already in process in the emergency room or started in the catheterization lab would provide the same reperfusion benefits as those described above when blood flow is restored to the ischemic tissues. Additional benefits in the catheterization lab would be realized when sequence modified CGRP infusion therapy locally dilates coronary blood vessels, decreases the incidence of vasospasms and no-reflow during procedures, increases renal blood flow, and assists in preventing platelet aggregation and smooth muscle cell proliferation at the acute time points (<24 hours) following PTCA. Currently, Reopro™ or Integrillin™ is administered in advance or during PTCA procedures to halt platelet aggregation and reduce the incidence of restenosis in the long-term (>48 hours). sequence modified CGRP infusion therapy would augment these current restenosis therapies by elevating the therapeutic benefits of preventing reperfusion injury, as well as inhibiting platelet aggregation and smooth muscle cell proliferation in the acute-term (<24, hours).

### Coronary Artery Bypass Surgery (CABG)

Whether CABG is performed as an emergency procedure or as elective surgery, sequence modified CGRP infusion therapy would provide all of the benefits stated above with respect to acute MI treatment and PTCA procedures. As a result, a CABG procedure could potentially experience even great positive outcomes and fewer acute-term complications.

### Coronary Care Unit (CCU) Recovery

Sequence modified CGRP infusion therapy in CCU patients would maximize the ability of sequence modified CGRP to reduce infarction size and promote cardiac tissue salvage. Whether the therapy was initiated in the emergency room, the cauterization lab, the operating room, or the CCU, recovery and healing process will begin in the CCU where sequence modified CGRP can be administered over the course of several days, and the long-term benefits of sequence modified CGRP infusion therapy will realized.

### Renal Function

This invention further provides methods for improving renal blood flow and glomerular filtration in a patient suffering from diminished renal function comprising administering sequence modified CGRP to a patient in need thereof in a manner effective to improve renal blood flow and/or glomerular filtration (see International Pub. Nos. WO 2005/070444 A2 and WO 2005/067890 A2, the disclosure of which are incorporated herein by reference in their entirety). Preferably administration is according to any of the above-describe dosing regimes for treating HF. As used herein, the term “improved renal function” includes increased glomerular filtration, increased renal blood flow and decreased serum levels of urea and creatinine.

### Angina

Angina pectoris, commonly known as angina, is chest pain due to ischemia (a lack of blood and hence oxygen supply) of the heart muscle, generally due to obstruction or spasm of the coronary arteries (the heart's blood vessels). Coronary artery disease, the main cause of angina, is due to atherosclerosis of the cardiac arteries. The term derives from the Greek ankhon (“strangling”) and the Latin pectus (“chest”), and can therefore be translated as “a strangling feeling in the chest”.

It is common to equate severity of angina with risk of fatal cardiac events. There is a weak relationship between severity of pain and degree of oxygen deprivation in the heart muscle (i.e. there can be severe pain with little or no risk of a heart attack, and a heart attack can occur without pain).

Worsening (“crescendo”) angina attacks, sudden-onset angina at rest, and angina lasting more than 15 minutes are symptoms of unstable angina (usually grouped with similar conditions as the acute coronary syndrome). As these may herald myocardial infarction (a heart attack), they require urgent medical attention and are generally treated as a presumed heart attack. (See http://en.wikipedia.org/wiki/Angina_pectoris.)

A patient suffering from angina be treated with a dose of sequence modified CGRP according to any of the methods of this invention until one or more symptoms of angina has diminished, and subsequently can be put on a sequence modified CGRP maintenance dosing regime.

### Sexual Dysfunction

The generation of penile and clitoral erections, vaginal and labial engorgement, and vaginal lubrication are dependent on adequate blood flow to vascular beds that feed these organs. Both smooth muscle relaxation of the corpora cavemosa as well as vasodilation of genital arterial vessels mediate the physiological response.

One of the etiologies of erectile dysfunction is inadequate genital arterial inflow. If there is an inappropriate narrowing in the supporting vasculature that is not associated with an increase in perfusion pressure, blood flow into the organs at maximum dilation may be reduced and thus insufficient for the generation of an erection. There also is increasing recognition that erectile dysfunction, although associated with, may appear prior to the onset of clinical signs of cardiovascular disease and therefore may be an early harbinger of progressing changes

In females, clitoral erectile insufficiency or reduced clitoral arterial flow may be caused by cardiac insufficiency, atherosclerosis, medication, diabetes mellitus, smoking, certain sexually transmitted diseases, nerve damage may have a negative effect on physiological, or age-related causes, among other factors. Reduced clitoral arterial flow may lead to fibrosis of the clitoral cavemosa and reduced clitoral physiological function.

Certain medication can also interfere with sexual arousal. Antidepressants, antihypertensives, and antihistamine medications are commonly associated with adverse sexual side effects. The direct physiological effects of these drugs interfere with the processes involved in sexual excitement.

Most of the treatments for sexual arousal disorders are still in the experimental stages, although a variety of products are being evaluated for their effectiveness in increasing blood flow to the genitalia and facilitating lubrication. Several vasodilator creams are being tested to measure their ability to improve sexual arousal. These creams work by expanding the arteries to increase blood flow to genital tissue. A number of oral medications are being investigated as well, including Viagra and related drugs, “natural” supplements such as DHEA and yohimbine, dopamine agonists, and drugs that stimulate the sympathetic nervous system. These drugs work by promoting blood flow, stimulating certain components of the nervous system, or a combination of both. Because most of these studies are fairly recent (or ongoing), there is not yet an FDA-approved medication for female sexual arousal disorder.

The present invention provides a method for counteracting male impotence or female sexual arousal disorder comprising: applying topically to the penis of a male mammal or to the genital area of a female mammal a pharmaceutical preparation of sequence modified calcitonin gene-related peptide (CGRP) which has been chemically bound to a naturally occurring polyunsaturated fatty acid.

Where sequence modified CGRP is used to treat impotence, topical application directly on the penis in the form of a cream is preferred. Any pharmaceutical preparation may be used, in particular a preferred cream being Aquaphore, which is commercially available from Beiersdorf Inc., Norwalk, Conn. The concentration of sequence modified CGRP in the cream should range from about 1-3 nmol, and in one preferred embodiment be about 2.5 nmol/ml. After topical application of about 0.5-1 ml of the sequence modified CGRP cream and gentle rubbing, the sequence modified CGRP ester will readily penetrate through the skin and accumulate in the penile corpora. Naturally occurring esterases in the subcutaneous tissues will then release active sequence modified CGRP into the local environment from the fatty acid conjugate. The result is rapid and sustained vasodilation of the blood vessels responsible for penile erection. The effect of the sequence modified CGRP should last between about 10-15 minutes. The local application may be repeated, if necessary, to result in another sustained erection lasting about 10-15 minutes. Persons of skill in the art can adjust the foregoing parameters to locally apply sequence modified CGRP to other sites in the body.

The present invention provides a method and composition for the treatment of female sexual arousal disorder (FSAD). Where sequence modified CGRP is used to treat FSAD, topical application of sequence modified calcitonin gene-related peptide (CGRP) conjugated to a hydrophobic agent directly to the female genitalia in the form of a cream is preferred.

When locally applied or infused, the effects of sequence modified CGRP are limited to a local vascular area. Virtually no systemic effects are induced, making sequence modified CGRP extremely safe and effective. Particularly preferred methods of administering of hydrophobic-sequence modified CGRP and/or other vasoactive agents for the treatment of female sexual dysfunctions are topical application, or transdermal application, by inhalation or by the use of suppositories.

After topical application of about 0.1-0.5 ml of the sequence modified CGRP cream to the clitoral area and gentle rubbing, the sequence modified CGRP ester will readily penetrate through the skin and accumulate. Naturally occurring esterases in the subcutaneous tissues will then release active sequence modified CGRp into the local environment from the fatty acid conjugate. The result is rapid and sustained vasodilation of the blood vessels responsible for clitoral erection. The effect of the sequence modified CGRP should last between about 20-45 minutes. The local application may be repeated, if necessary, to result in a continued state of arousal lasting about 20-45 minutes.

Also contemplated herein is the treatment of patients harboring specific genetic polymorphisms that confer altered therapeutic response to the sequence modified CGRPs. In some embodiments, the polymorphisms are present in the CGRP receptor.

The invention will be further described by reference to the following detailed example, which is given for illustration of the invention, and is not intended to be limiting thereof.

### Example 1

### Methods

The following peptides were synthesized:

Peptide III:
(SEQ ID NO: 5)
Cyc-ACDTATCVTHRLAGLLSRSGGVVKSNFVPTNVGSKAF-NH₂
Peptide V:
(SEQ ID NO: 7)
Cyc-ACNTATCVTHRLAGLLSRSGGVVKSNFVPTNVGSKAF-NH₂
Peptide IX:
(SEQ ID NO: 11)
Cyc-ACDTATCVTHRLADLLSRSGGVVKSNFVPTNVGSKAF-NH₂

All peptides were synthesized on an Applied Biosystems 433 instrument using standard solid phase peptide chemistry with FMOC protected amino acids on Rink amide resin. Amino acid activation and couplings were carried out with HBTU/HOBt (0.45 M) and DIEA. FMOC groups were removed using 20% piperidine in DMF. The linear sequence was then cleaved and deprotected with TFA containing 5% water, 5% thioanisol, 5% ethylmethylsulfide, and 5% ethanedithiol. The peptide was then precipitated into ether, spun, and dried. The linear sequence was purified by reverse phase HPLC on a C18 column and eluted with acetonitrile-water buffers containing 0.1% TFA. The purified peptide was then cyclized using 0.1 M ammonium bicarbonate buffer overnight. The cyclized peptide was purified by reverse phase HPLC to 95% purity on a C18 column and eluted with acetonitrile-water buffers containing 0.1% TFA. Pure fractions were pooled and lyophilized. The cyclized peptide was reconstituted in 25 mM ammonium acetate buffer and lyophilized. The reconstitution and lyophilization steps were repeated twice to exchange to an acetate salt. HPLC data was run on a 5 micron analytical column and eluted with water-acetonitrile buffers containing 0.1% TFA. Molecular weight was confirmed by MALDI-TOF analysis.

The table below summarizes the analytical data for all three peptides:

TABLE 6

| | | | | | Measured |
|---|---|---|---|---|---|
| | HPLC^{a} | | Peptide | Calculated | mass |
| Peptide | retention | Final | content | mass | (M + H)⁺ |
| Name | time | Purity | (AAA) | (M + H)⁺ | MALDI |
| Peptide III | 34.6 min^{b} | 98% | 78.9% | 3763 | 3763 |
| Peptide V | 18.3 min^{c} | 96% | 75.0% | 3762 | 3762 |
| Peptide IX | 20.6 min^{c} | 99% | 73.2% | 3821 | 3821 |

^{a}Analytical HPLC was performed on a YMC-Pack ODS, 150 mm × 4.6 mm, packed with C18 silica, 5 μM, 120 Å with a flow rate of 1.0 mL/min. Buffer A: water with 0.1% TFA, Buffer B: acetonitrile with 0.1% TFA.
^{b}Gradient: 1-40% B over 45 minutes
^{c}Gradient: 1-50% B over 25 minutes

The peptide disulfide bond formation was confirmed by NMR and MS/MS sequencing.

All test articles were analyzed for purity and had maintained 95+% purity since their synthesis and formulation into the buffered IV solution at Ph 4.

cDNA Constructs

The expression vectors for RAMP1, RAMP2, RAMP3, CRLR, CRE-Luc and SRE-Luc were constructed as previously described (McLatchie L. M., et al. (1998) Nature 393:333-339). Human CRLR, RAMP1, and RAMP2 full-length cDNAs were cloned by RT-PCR using RNA obtained from HEK293T cells and CRLR, RAMP1, and RAMP2-specific primers. Human RAMP3 full-length cDNAs were cloned by RT-PCR using RNA from human kidney and RAMP3 specific primers. All of the full-length cDNAs were subcloned into pcDNA3.1 expression vectors.

Cell Culture and cDNA Transfection

Human embryonic kidney 293T cells (HEK293T) were maintained in Dulbecco's modified Eagle's medium supplemented with 10% fetal calf serum, 2 mM glutamine, 100 μg/ml penicillin, 100 units/ml streptomycin, at 37° C. in a humidified atmosphere of 95% air and 5% CO2. Transient transfections were performed using calcium phosphate co-precipitation (Mellon P. L., et al. (1981) Cell 27:279-288). Cells in six-well plates or 100-mm Petri dishes were grown to 70% confluence and transfected with 0.1 μg of DNA for CRLR, 0.05 μg for RAMPs or 1 μg for CRE-Luc and SRE-Luc per cm². The total amount of DNA transfected in each condition was kept constant by adding empty pcDNA3.1 vector when needed. Twenty hours after transfection, the medium was replaced with fresh medium containing 5 mM sodium butyrate for 24 h and then replaced for an additional 24 h with fresh medium without sodium butyrate. All of the experiments were performed 72 h after transfection to allow correct glycosylation and cell surface targeting of CRLR and RAMPs.

After exposure to various concentrations of CGRP or the CGRP analogs, the cells were lysed and assayed for CRE- or SRE-luciferase activity using the luciferase assay system (Promega), and luminescence was measured using a 96-well luminometer. Increased CRE-luciferase activity was indicative that CGRP or the CGRP analog increased cAMP associated cellular responses. Increased SRE-luciferase activity was indicative that CGRP or the CGRP analog increased [Ca2+] and associated cellular responses.

### Results

### In Vivo

The three analogs of h-α-CGRP evaluated were [Ser²⁵] h-α-CGRP (“III”), [Asn³, Ser²⁵] h-α-CGRP (“V”) and [Asp¹⁴, Ser²⁵] h-α-CGRP (“IX”). These analogs correspond to h-β-CGRP denoted as [Asp³, Val²²] h-β-CGRP (“III”), [Val²²]h-β-CGRP h-β-CGRP (“V”) and [Asp³, Asp¹⁴, Val²²] h-β-CGRP (“IX”).

In the sedated Non Human Primate, each of the three analog peptides unexpectedly demonstrated greater reductions in mean arterial pressure (MAP), diastolic Blood pressure (DBP) and systolic blood pressure (SBP) and a reduced increase in heart rate (HR) when compared to the control, Calcitonin Gene Related Peptide (CGRP). Each test article and control were administered IV for 5 minutes at a dose of 29/ng/kg/min. Compared to control CGRP, the MAP reductions ranged from 20 to 110% greater, the DBP ranged from 5 to 160% greater, the SBP ranged from 5 to 82% and the reduction in HR increase ranged from 22 to 82% with the analogs. Increases in blood pressure reductions did not parallel the increased reductions in heart rate increases compared to the CGRP control.

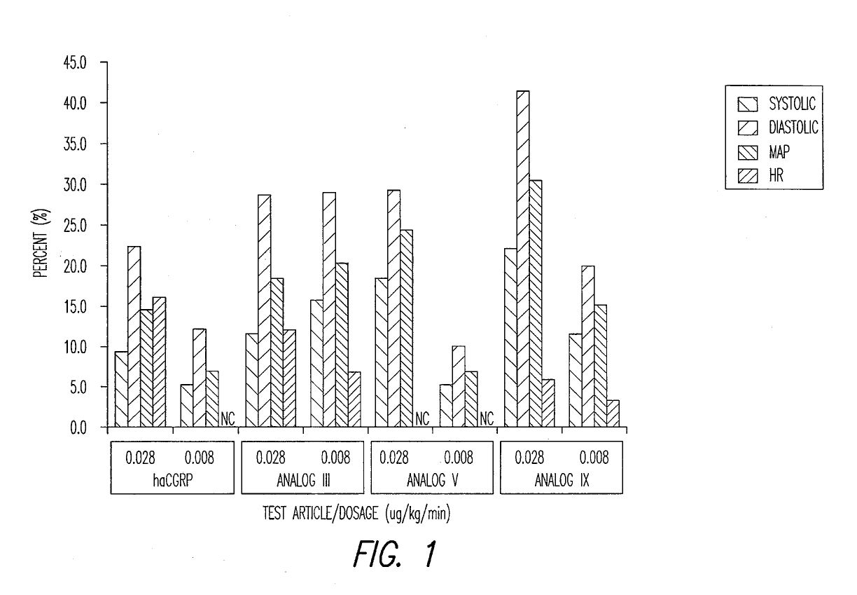

All analogs and control CGRP lowered mean arterial pressure (MAP), systolic pressure (SP) and diastolic pressure (DP) at all doses tested. Heart rate (HR) increases were found for alpha CGRP at the 28 ng dose but not at the 8 ng dose. Peptides III and IX increased heart rate but less than CGRP at the 28 ng dose. Unexpectedly, Compound V demonstrated no increase in heart rate at any of the tested doses suggesting importance to the presence of asparagine at position 3 and serine at position 25. See Table 3 and FIG. 1. The work of Gardiner showed that infusions of human alpha CGRP in rats caused more significant hemodynamic effects compared to human beta CGRP. In addition human alpha CGRP at 19 ng/kg/min resulted in tachycardia accompanied by a fall in blood pressure but human beta CGRP caused tachycardia with no significant hypotension. Therefore, analog V was expected to be more like beta CGRP because 2 of the 3 substitutions were from the beta form, but this was not the case. Thus, the new sequence modified CGRPs of the invention provide improved activity and a decrease in negative effects compared to native CGRP.

### In Vitro

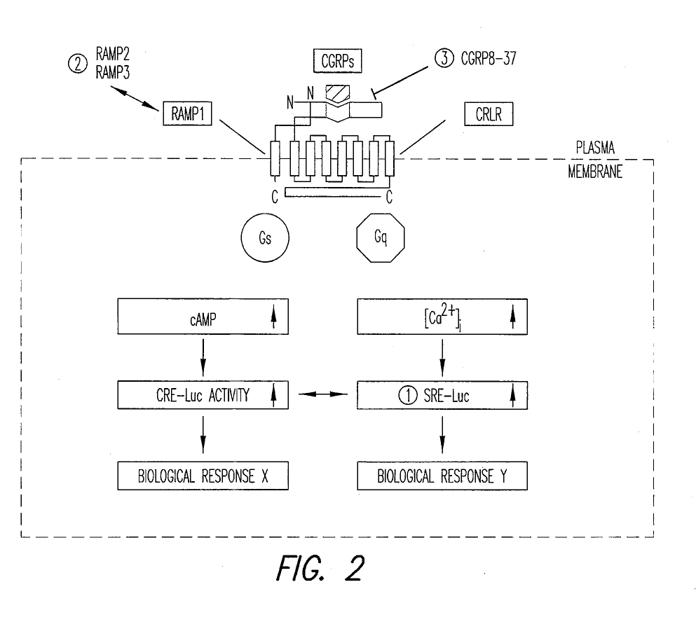

Cell culture studies conducted at the University of Kansas Kidney Institute have demonstrated that all three analogs bind to the same CRLR/RAMP1 receptor. This is a very important finding since the CRLR/RAMP1 receptor is highly specific for CGRP and is not known to bind any other peptide. The mechanism of action for CGRP at the CRLR/RAMP1 receptor is known to up-regulate cAMP through the gₛ pathway, and to a lesser extent [Ca²⁺] through the g_{q} pathway. FIG. 2 shows the two known cellular pathways regulated by CGRP binding to the CRLR/RAMP1 receptor. It is the cAMP pathway that is the primary mechanism of action of CGRP for vasodilation, cardio/renoprotection and immunomodulation.

The three CGRP analogs were evaluated in a CRE-Luciferase assay and compared to h-α-CGRP and h-β-CGRP as controls. Analogs III, V and IX were as equally active and potent as h-α-CGRP and h-β-CGRP in binding to the CGRP receptor. Analog IX was more potent at a concentration of 10⁻¹⁰ molar. Receptors were saturated at 10⁻⁷ molar for the analogs and with h-α-CGRP and h-β-CGRP demonstrating similarities in receptor binding. Analog IX was 1.5 fold higher in activity at 10⁻¹⁰ molar than analogs III and V as well as h-α-CGRP and h-β-CGRP, paralleling the drop in MAP, SP and DP in vivo, confirming the importance of substituting Asp at position 14 for Gly.

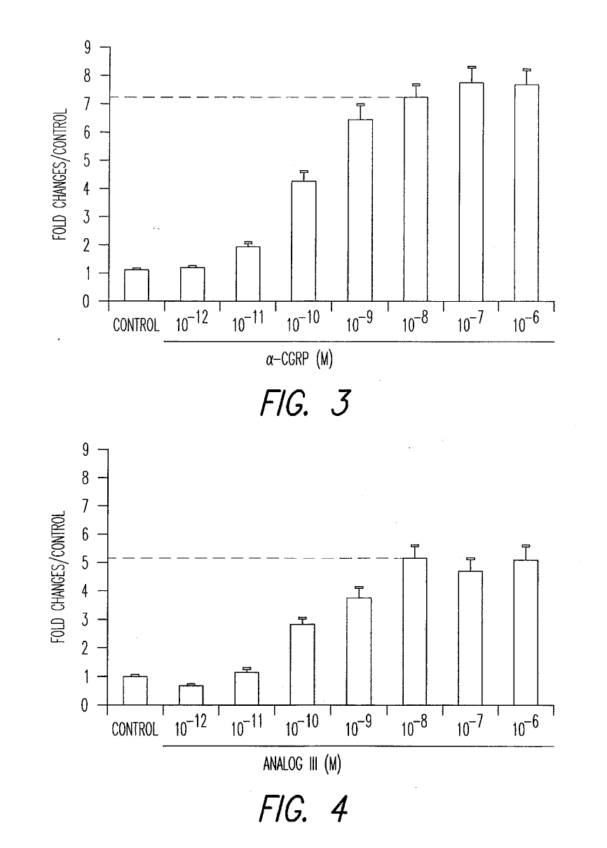

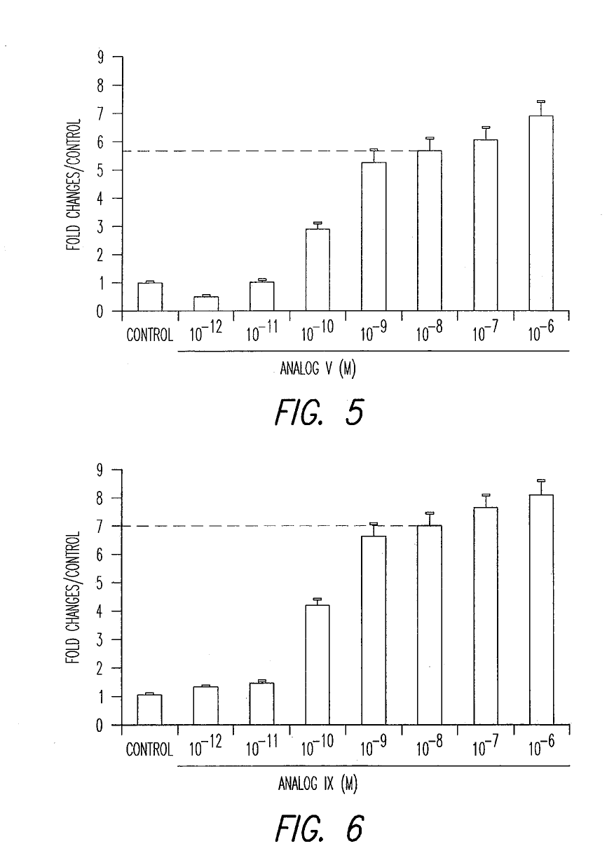

The data illustrated in FIGS. 3-6 shows that the analogs have similar binding activity for RAMP1 as native CGRP, initiating cAMP activity in a dose responsive manner. The data further confirms the physiologic responses observed in the non human primate studies reported above.

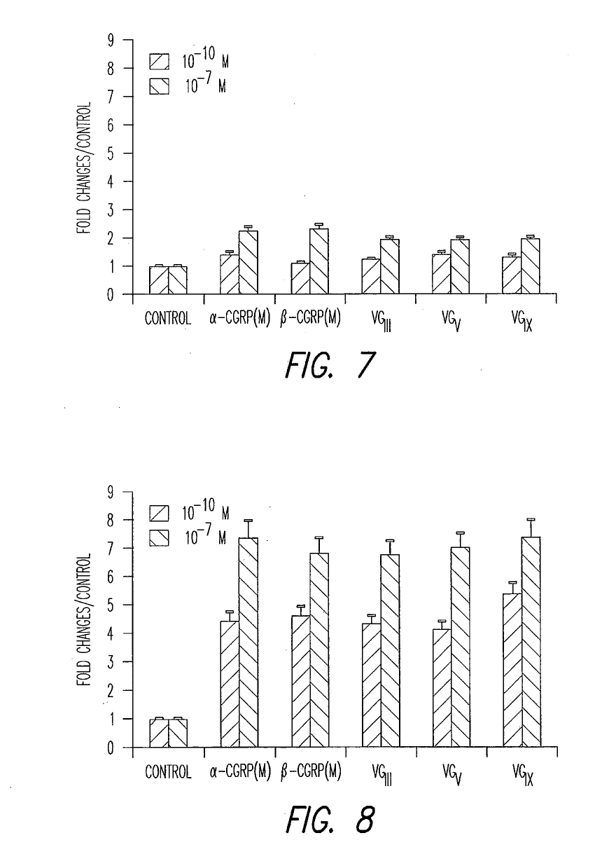

Studies specific for [Ca²⁺] were conducted to further understand the mechanism of action of the analogs relative to CGRP. Ca²⁺ is thought to be responsible for the mild inotropic actions of CGRP but is poorly understood. These studies used SRE-luciferase to isolate the Ca²⁺ pathway. As shown in FIG. 7 CGRP and the analogs had minimal activity in inducing the Ca²⁺ pathway and higher concentrations were equally inactive.

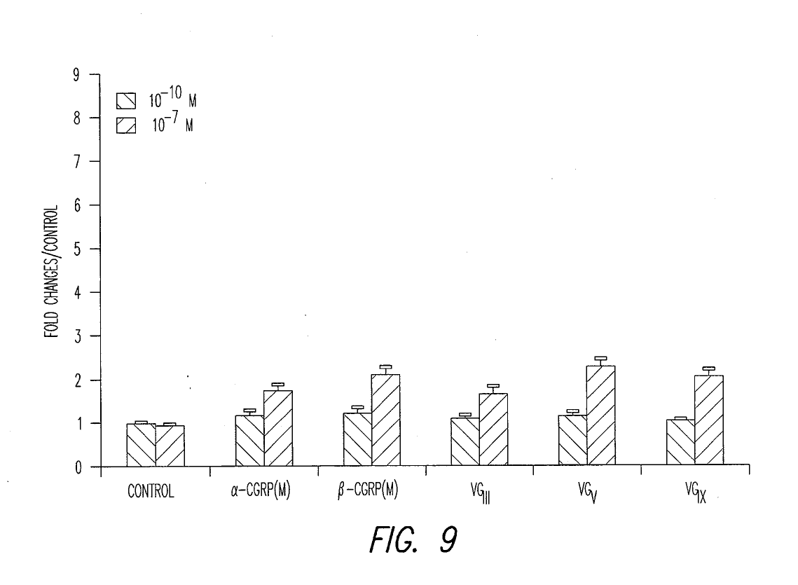

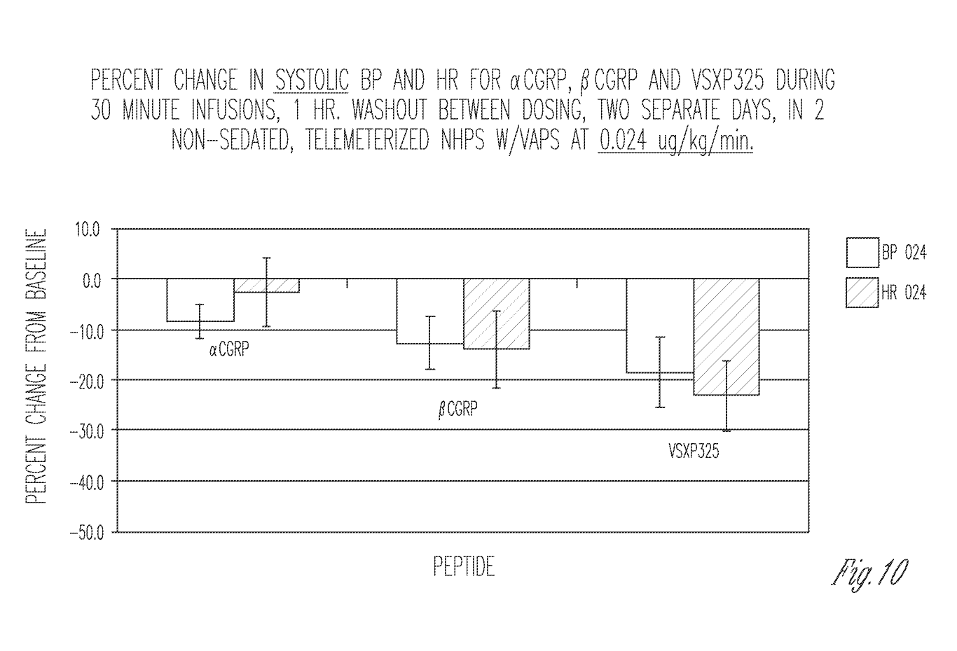

To assess whether the analogs had the same specificity to RAMP1 receptor and the endogenous CGRPs, a study was conducted where RAMP2 and RAMP3 were transfected into the same cell line. In FIGS. 8 and 9, both CGRP and the analogs had minimal to no binding affinity to either the RAMP2 or RAMP3 receptors.

These cell culture studies confirm that the CGRP agonist analogs have the same receptor binding affinity, binding specificity, and mechanisms of action as endogenous hαCGRP and hβCGRP.

### Example 2

### Methods

Peptides were synthesized as in Example 1. The test articles of human α-CGRP, β-CGRP and VSXP325 were supplied as stock solutions and formulated into dosing solutions for intravenous infusions at 0.016 ug/kg/min and 0.024 ug/kg/min. Two non-naïve, telemeterized, male non-human primates (NHP “a” and “b”) were used for the test article(s) dosing regimen. Test articles included α-CGRP, β-CGRP and Peptide V (also known as VSXP325, Structure V, Peptide V, Analog V and Compound V). Following a stabilized baseline period of approximately 30 minutes, one of the test article(s) was administered as an intravenous infusion via a femoral vein vascular access port (VAP) using a Pegasus PCA infusion pump for a period of 30 or 60 minutes. A minimum period of 30 minutes was allotted for the hemodynamic parameters to return to baseline prior to the next test article infusion dose. The process was repeated for each of the stated test articles. The same procedure was followed for the 1 hour infusion with 1 hour allotted for parameters to return to base line.

TABLE 7

| | | | Test | Dose | Nominal | |
|---|---|---|---|---|---|---|
| Dose | | Route of | Substance | Volume | Concentration | Nominal Dose |
| Days. | n | Administration | Dosing Order | (mL/kg) | (μg/mL) | (μg/kg/min) |
| 1 | 2 | Intravenous | α-CGRP, | 1.0 | 4.8 | 0.016 |
| | | | VSXP325 and | | | |
| | | | β-CGRP | | | |
| 2^{a} | 2 | Intravenous | α-CGRP, | 1.0 | 7.2 | 0.024 |
| | | | VSXP325 and | | | |
| | | | β-CGRP | | | |
| 3^{b} | 2 | Intravenous | α-CGRP, | 1.0 | 7.2 | 0.024 |
| | | | VSXP325 and | | | |
| | | | β-CGRP | | | |

^{a}Dose day 2 may be a repeat of the same dose for confirmation of the results.
^{b}Dose day 3 may be the same dose as day 1 but for a 1 hr infusion.

The non-human primates were previously surgically prepared with telemetry transducers, type TL11M2-D70-PCT (Data Sciences International). The acquisition and analysis was done using a Ponemah (Version 4.8 or higher) data acquisition and analysis system installed on the hard drive of a computer. The computer was equipped with disk drive for file back up on readable write-once only optical media (CD-R or DVD-R).

Prior to the first dose administration, baseline data was acquired. For each subsequent dose, data was collected for approximately 30 min postdose. Prior to the next dose, the hemodynamic parameters were allowed to return to a stable baseline±10%.

Systolic blood pressure (SYS or SBP), diastolic blood pressure (DBP), mean arterial pressure (MAP), heart rate (HR), and all lead II ECG variables were recorded. The telemetry data files were transferred to an Excel (MicroSoft® Version 2003) spreadsheet. The following variables from the lead II ECG were measured: PR interval, QRS duration, QT interval, RR interval, and QTcF interval (calculated as

$$QTcF = \frac{QT}{\sqrt[3]{RR}} ) .$$

A visual inspection of the entire collected ECG waveform for disturbances in rhythm and waveform morphology was performed. The data for selected time points were analyzed and assessed on completion of data acquisition and were the average of at least 30 beats (ABP and ECG) of data at the selected analysis time, when possible. The time points for both hemodynamic and ECG data analysis were an initial 30 min baseline prior to the first dose, at a 10 min during each infusion postdose interval relative to end of each infusion, and time points prior to the next test article infusion for each test compound.

### Results

This study assessed the cardiovascular effects of the CGRP analog Peptide V (also known as VSXP325, Structure V, Analog V and Compound V) and β-CGRP when compared to the positive control compound human α-CGRP on blood pressure, heart rate, and lead II electrocardiogram in two non-sedated, non-naïve cynomolgus monkeys. Non-human primates were selected because they have the same RAMP 1-CGRP receptor complex as humans.

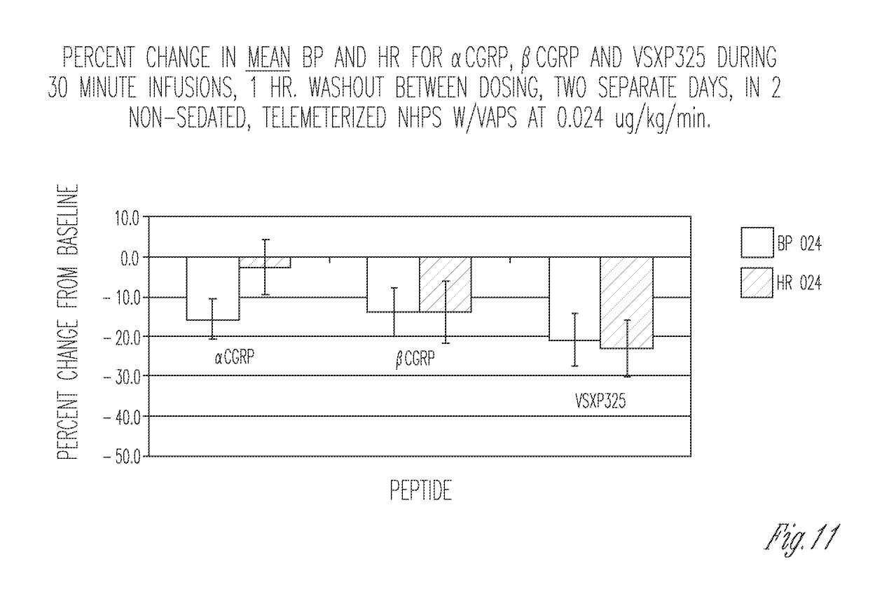

The maintenance of pre-administration heart rate, and in some cases, reduction in heart rate, with βCGRP and Analog V (VSXP325) was an unexpected effect. Tables 8-25 and FIGS. 10-13 show specific values for various parameters including percent change in systolic BP and HR. “Avgdev”=average deviation. “a 1”=non-human primate “a” on dose day 1. “a 2”=non-human primate “a” on dose day 2. “b 1”=Non-human primate “b” on dose day 1. “b 2”=Non-human primate “b” on dose day 2, and so on.

TABLE 8
Baseline values of each non-human primate (NHP) before infusion
(reference point for data in Tables 9-16).

| | Sys | Mean | HR |
|---|---|---|---|
| NHP a | 143.7 | 121.5 | 163.4 |
| NHP b | 138.1 | 106.2 | 221.7 |

TABLE 9
Percent change in systolic BP during 30 minute
infusions of 0.024 ug/kg/min, 1 hour washout between
dosing, two separate days in two NHPs.

| | a 2 | b 2 | a 3 | b 3 | BP | avgdev |
|---|---|---|---|---|---|---|
| αCGRP | −12.2 | −8.1 | −11.6 | −1.8 | −8.4 | 3.5 |
| βCGRP | −7.4 | −8.1 | −12.2 | −23.2 | −12.7 | 5.2 |
| VSXP325 | −16.2 | −27.2 | −6.8 | −23.9 | −18.5 | 7.0 |

TABLE 10
Percent change in HR during 30 minute infusions of 0.024 ug/kg/min,
1 hour washout between dosing, two separate days in two NHPs.

| | a 2 | b 2 | a 3 | b 3 | HR | avgdev |
|---|---|---|---|---|---|---|
| αCGRP | −4.2 | 6.3 | −14.8 | 2.0 | −2.7 | 6.8 |
| βCGRP | 1.2 | −21.3 | −13.6 | −22.0 | −13.9 | 7.7 |
| VSXP325 | −19.8 | −13.1 | −22.2 | −37.3 | −23.1 | 7.1 |

The data from Tables 8-10 is shown graphically in FIG. 10. The VSXP325 peptide reduced BP and HR to a greater extent than αCGRP and βCGRP.

TABLE 11
Percent change in systolic BP during 30 minute infusions of 0.016
ug/kg/min, 1 hour washout between dosing, two separate days in
two NHPs.

| | a1 | b1 | BP | avgdev |
|---|---|---|---|---|
| αCGRP | −9.7 | −20.3 | −15.0 | 5.3 |
| βCGRP | −12.6 | −28.9 | −20.8 | 8.2 |
| VSXP325 | −5.6 | −23.9 | −14.8 | 9.2 |

TABLE 12
Percent change in HR during 30 minute infusions of 0.016 ug/kg/min,
1 hour washout between dosing, two separate days in two NHPs.

| | a1 | b1 | HR | avgdev |
|---|---|---|---|---|
| αCGRP | −4.9 | −16.3 | −10.6 | 5.7 |
| βCGRP | −14.1 | −43.9 | −29.0 | 14.9 |
| VSXP325 | −3.6 | −11.5 | −7.6 | 4.0 |

TABLE 13
Percent change in mean BP during 30 minute infusions
of 0.024 ug/kg/min, 1 hour washout between dosing,
two separate days in two NHPs.

| | a 2 | b 2 | a 3 | b 3 | BP | avgdev |
|---|---|---|---|---|---|---|
| αCGRP | −15.2 | −25.7 | −16.0 | −6.4 | −15.8 | 5.0 |
| βCGRP | −8.8 | −6.7 | −16.8 | −23.4 | −13.9 | 6.2 |
| VSXP325 | −20.0 | −28.6 | −8.8 | −26.4 | −21.0 | 6.6 |

TABLE 14
Percent change in mean HR during 30 minute infusions
of 0.024 ug/kg/min, 1 hour washout between dosing,
two separate days in two NHPs.

| | a 2 | b 2 | a 3 | b 3 | HR | avgdev |
|---|---|---|---|---|---|---|
| αCGRP | −4.2 | 6.3 | −14.8 | 2.0 | −2.7 | 6.8 |
| βCGRP | 1.2 | −21.3 | −13.6 | −22.0 | −13.9 | 7.7 |
| VSXP325 | −19.8 | −13.1 | −22.2 | −37.3 | −23.1 | 7.1 |

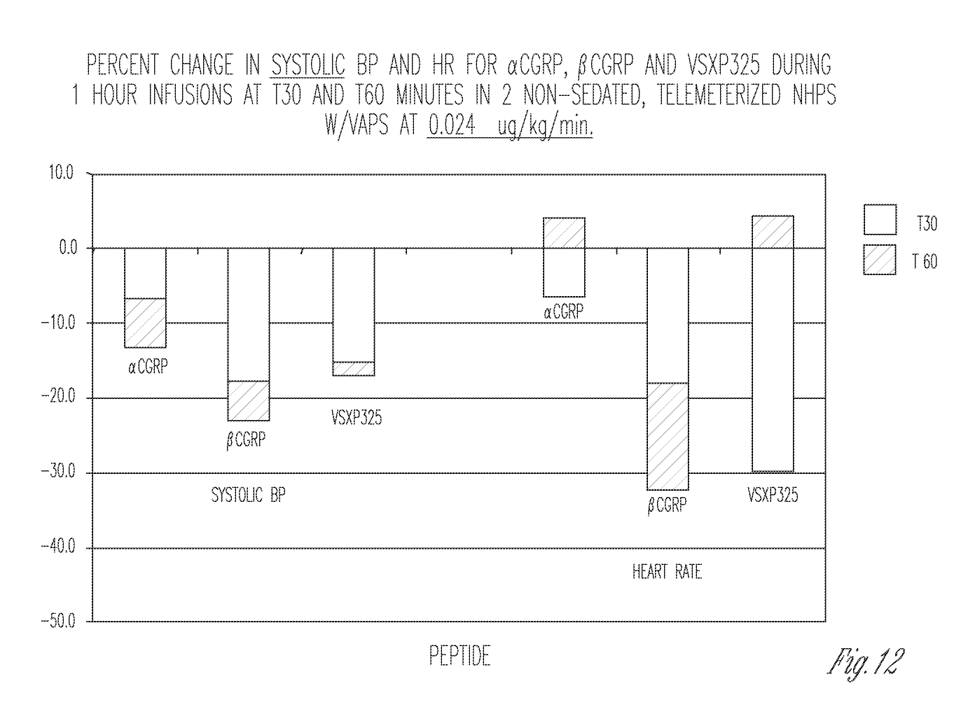

The data from Tables 8, 13 and 14 is shown graphically in FIG. 11. VSXP325 (Peptide V) reduced both BP and HR to a greater extent than the positive control αCGRP.

TABLE 15
Percent change in mean BP during 30 minute infusions of 0.016 ug/
kg/min, 1 hour washout between dosing, two separate days in two NHPs.

| | a1 | b1 | BP | avgdev |
|---|---|---|---|---|
| αCGRP | −6.6 | −29.8 | −18.2 | 11.6 |
| βCGRP | −13.2 | −36.4 | −24.8 | 11.6 |
| VSXP325 | −0.8 | −29.8 | −15.3 | 14.5 |

TABLE 16
Percent change in mean HR during 30 minute infusions of 0.016 ug/
kg/min, 1 hour washout between dosing, two separate days in two NHPs.

| | a1 | b1 | HR | avgdev |
|---|---|---|---|---|
| αCGRP | −4.9 | −16.3 | −10.6 | 5.7 |
| βCGRP | −14.1 | −43.9 | −29.0 | 14.9 |
| VSXP325 | −3.6 | −11.5 | −7.6 | 4.0 |

TABLE 17
Baseline values of each non-human primate (NHP) before infusion
(reference point for data in Tables 18-25.

| | Sys | Mean | HR |
|---|---|---|---|
| NHP a | 143.7 | 121.5 | 163.4 |
| NHP b | 138.1 | 106.2 | 221.7 |

TABLE 18
Percent change in systolic BP at 30 minutes during 1 hour infusion
of 0.024 ug/kg/min.

| | a3 | b3 | T30 | avgdev |
|---|---|---|---|---|
| αCGRP | −11.6 | −1.8 | −6.7 | 4.9 |
| βCGRP | −12.2 | −23.2 | −17.7 | 5.5 |
| VSXP325 | −6.8 | −23.9 | −15.4 | 8.6 |

TABLE 19
Percent change in systolic BP at 60 minutes during 1 hour infusion
of 0.024 ug/kg/min.

| | a3 1 hr | b3 1 hr | T60 | avgdev |
|---|---|---|---|---|
| αCGRP | −2.3 | −10.7 | −6.5 | 4.2 |
| βCGRP | −0.8 | −10.1 | −5.4 | 4.7 |
| VSXP325 | −5.8 | 2.8 | −1.5 | 4.3 |

TABLE 20
Percent change in HR at 30 minutes during 1 hour infusion of
0.024 ug/kg/min.

| | a3 | b3 | HR T30 | avgdev |
|---|---|---|---|---|
| αCGRP | −14.8 | 2.0 | −6.4 | 8.4 |
| βCGRP | −13.6 | −22.0 | −17.8 | 4.2 |
| VSXP325 | −22.2 | −37.3 | −29.8 | 7.6 |

TABLE 21
Percent change in HR at 60 minutes during 1 hour infusion of
0.024 ug/kg/min.

| | a3 | | | |
|---|---|---|---|---|
| | 1 hr | b3 1 hr | HR T60 | avgdev |
| αCGRP | 9.3 | −1.0 | 4.2 | 5.2 |
| βCGRP | −10.7 | −18.1 | −14.4 | 3.7 |
| VSXP325 | −5.8 | 14.5 | 4.4 | 10.2 |

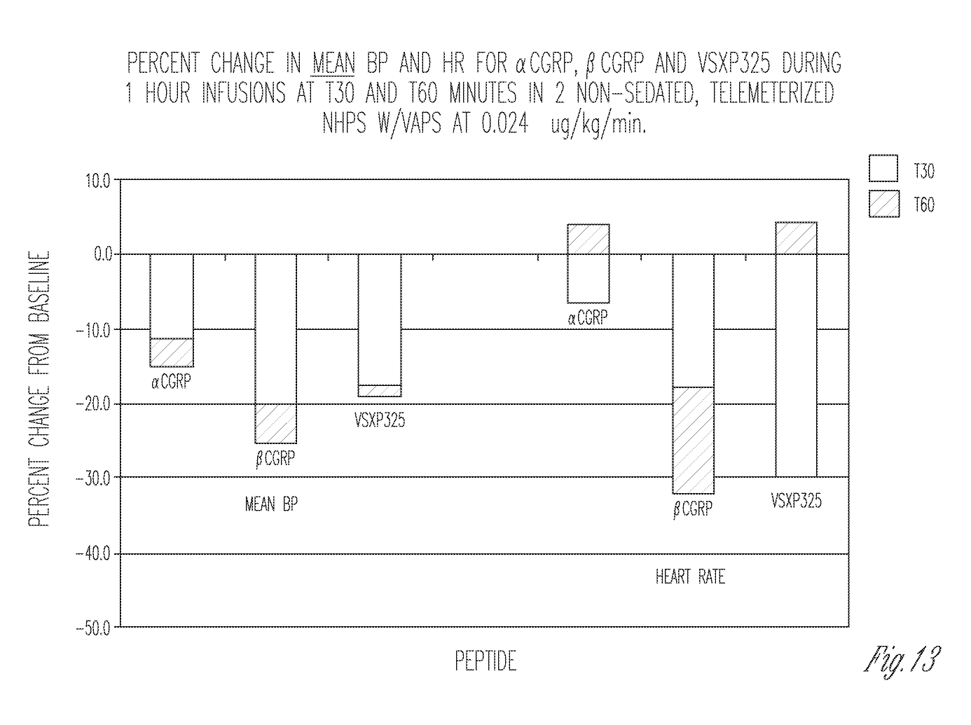

The data from tables 17-21 are shown graphically in FIG. 12. Measurements at 30 and 60 minutes illustrate the cumulative action of αCGRP, βCGRP and VSXP325 (Peptide V) during 1 hour infusion. Most of the effects happen in the first 30 minutes. Heart rate continues to drop after 30 minutes for beta CGRP, even though Systolic BP has nearly stabilized. All returned to base line within 20-50 mins.

TABLE 22
Percent change in mean systolic BP at 30 minutes during 1 hour
infusion of 0.024 ug/kg/min.

| | a3 | b3 | BP T30 | avgdev |
|---|---|---|---|---|
| αCGRP | −16.0 | −6.4 | −11.2 | 4.8 |
| βCGRP | −16.8 | −23.4 | −20.1 | 3.3 |
| VSXP325 | −8.8 | −26.4 | −17.6 | 8.8 |

TABLE 23
Percent change in mean systolic BP at 60 minutes during 1 hour
infusion of 0.024 ug/kg/min.

| | a3 1 hr | b3 1 hr | BP T60 | avgdev |
|---|---|---|---|---|
| αCGRP | −1.0 | −6.8 | −3.9 | 2.9 |
| βCGRP | 0.0 | −10.7 | −5.4 | 5.4 |
| VSXP325 | −5.3 | 2.4 | −1.5 | 3.9 |

TABLE 24
Percent change in mean HR at 30 minutes during 1 hour infusion of
0.024 ug/kg/min.

| | a3 | b3 | HR T30 | avgdev |
|---|---|---|---|---|
| αCGRP | −14.8 | 2.0 | −6.4 | 8.4 |
| βCGRP | −13.6 | −22.0 | −17.8 | 4.2 |
| VSXP325 | −22.2 | −37.3 | −29.8 | 7.6 |

TABLE 25
Percent change in mean HR at 60 minutes during 1 hour infusion of
0.024 ug/kg/min.

| | a3 | | | |
|---|---|---|---|---|
| | 1 hr | b3 1 hr | HR T60 | avgdev |
| αCGRP | 9.3 | −1.0 | 4.2 | 5.2 |
| βCGRP | −10.7 | −18.1 | −14.4 | 3.7 |
| VSXP325 | −5.8 | 14.5 | 4.4 | 10.2 |

The data from Tables 17 and 22-25 are shown graphically in FIG. 13. Measurements at 30 and 60 minutes illustrate the cumulative action of αCGRP, βCGRP and VSXP325 (Peptide V) during 1 hour infusion. Mean arterial pressure drops 10 to 20% and steadies, and no change in heart rate over 24 hours is seen. Most of the effects happen in the first 30 minutes. Heart rate continues to drop after 30 minutes for beta CGRP, even though Systolic BP has nearly stabilized. All returned to base line within 20-50 mins and no toxic effects were observed.

### BIBLIOGRAPHY

Aiyar N, Disa J, Pullen M, and Nambi P. Receptor activity modifying proteins interaction with human and porcine calcitonin receptor-like receptor (CRLR) in HEK-293 cells. Mol Cell Biochem 224:123-133 (2001).
Aiyar N, Rand K, Elshourbagy N A, Zeng Z Z, Adamou J E, Bergsma D J, and Li Y. A cDNA encoding the calcitonin-gene-related peptide type-1 receptor. J Biol Chem 271:11325-11329 (1996).
Aldecoa A, Gujer R, Fischer J A, and Born W. Mammalian calcitonin receptor-like receptor/receptor activity modifying protein complexes define calcitonin gene-related peptide and adrenomedullin receptors in Drosophila Schneider 2 cells. FEBS Lett 471:156-160 (2000).
Anand I S, Gurden J, Wander G S, O'Gara P, Harding S E, Ferrari R, et al. Cardiovascular and hormonal effects of calcitonin gene-related peptide in congestive heart failure. J Am Coll Cardiol 1991; 17:208-217.
Banerjee S, Evanson J, Harris E, Lowe S, Thomasson K and Porter J. Identification of specific calcitonin-like receptor residues important for calcitonin gene-related peptide high affinity binding. BMC Pharmacology (2006), 6:9
Buhlmann, N., Leuthauser, K., Muff, R., Fischer, J. A. and Born, W. A receptor activity modifying protein (RAMP)-2-dependent adrenomedullin receptor is a calcitonin gene-related peptide receptor when coexpressed with human RAMP1. Endocrinology 140, 2883-2890 (1999).
Brain S D, Grant A D. Vascular actions of calcitonin gene-related peptide and adrenomedullin. Physiol Rev. 2004 July; 84(3):903-34.
Chiba, T. et. al., Calcitonin gene-related peptide receptor antagonist human CGRP-(8-37). Am. J. Physiol 256, E331-E335 (1989).
Conner A C, Hay D, Simms J, Howitt S, Schindler M, Smith D, Wheatley M, and Poyner D. A Key Role for Transmembrane Prolines in Calcitonin Receptor-Like Receptor Agonist Binding and Signaling: Implications for Family B G-Protein-Coupled Receptors. Mol Pharmacol 67:20-31, (2005).
Elshourbagy N A, Adamou J E, Swift A M, Disea J, Mao J, Ganguly S, Bergsma D J, and Aiyar N. Molecular cloning and characterization of the porcine calcitonin gene-related peptide receptor. Endocrinology 139:1678-1683 (1998).
Esfandyari T, Macnaughton W K, Quirion R, St Pierre S, Junien J L, and Sharkey K A. A novel receptor for calcitonin gene-related peptide (CGRP) mediates secretion in the rat colon: implications for secretory function in colitis. FASEB J 14:1439-1446 (2000).
Evans B, Rosenblatt M, Mnayer L, Oliver K, and Dickerson I. CGRP-RCP, a Novel Protein Required for Signal Transduction at Calcitonin Gene-related Peptide and Adrenomedullin Receptors. J. Biol. Chem. 275, No. 40, October 6, pp. 31438-31443, (2000).
Gardiner S M, Compton A M and Bennett T. Regional Hemodynamic Effects of Human α- and β-CGRP in Conscious Wistar Rats. Br. J. Pharmacol. 98, 1225-1232 (1989).
Gennari C, Nami R, Agnusdei D, Fischer J A. Improved cardiac performance with human calcitonin gene related peptide in patients with congestive heart failure. Cardiovasc Res 1990; 24:239 241.
Han, S P, et. al., Inhibition of periarterial nerve stimulation-induced vasodilation of the mesenteric arterial bed by CGRP(8-37) and CGRP receptor desensitization. Biochem Biophys res Commun 168, 786-791 (1990)
Husmann K, Sexton P M, Fischer J A, and Born W. Mouse receptor-activity modifying proteins 1, -2 and -3: amino acid sequence, expression and function. Mol Cell Endocrinol 162:35-43 (2000).
Kuwasako K, Kitamura K, Nagoshi Y, Cao Y N, Eto T. Identification of the human receptor activity-modifying protein 1 domains responsible for agonist binding specificity. J Biol. Chem. 2003 Jun. 20; 278(25):22623-30.
Mallee, J J, Salvatore, C A, LeBourdelles, B, Oliver, K R, Longmore, J, Koblan, K S, and Kane, S A Receptor activity-modifying protein 1 determines the species selectivity of non-peptide CGRP receptor antagonists. J. Biol. Chem. 277:14294-14298 (2002).
McLatchie, L. M., Fraser, N. J., Main, M. J., Wise, A., Brown, J., Thompson, N., Solari, R., Lee, M. G. and Foord, S. M. (1998). RAMPs regulate the transport and ligand specificity of the calcitonin-receptor-like receptor. Nature 393, 333-339.
Nuki C, Kawasaki H, Takasaki K and Wada A. Structure-Activity Study of Chicken Calcitonin Gene-Related Peptide (CGRP) on Vasorelaxation in Rat Mesenteric Resistance Vessels. Jpn. J. Pharmacol. 69, 99-105 (1994).
Oliver K R, Kane S A, Salvatore C A, Mallee J J, Kinsey A M, Koblan K S, Keyvan-Fouladi N, Heavens R P, Wainwright A, Jacobson M, et al. Cloning, characterization and central nervous system distribution of receptor activity modifying proteins in the rat. Eur J Neurosci 14:618-628 (2001).
Poyner D, Sexton P, Marshall I, Snith D, Quirion R, Born W, Muff R, Fischer J, and Foord S. International Union of Pharmacology. XXXII. The Mammalian Calcitonin Gene-Related Peptides, Adrenomedullin, Amylin, and Calcitonin Receptors. Pharmacol Rev 54:233-246, 2002.
Salvatore C, Hershey J, Corcoran H, Fay J, Johnston V, Moore E, Mosser S, Burgey C, Paone D, Shaw A, Graham S, Vacca J, Williams T, Koblan K, and Kane S. Pharmacological Characterization of MK-0974, a Potent and Orally Active CGRP Receptor Antagonist for the Treatment of Migraine. JPET Fast Forward. Published on Nov. 26, 2007 as DOI:10.1124/jpet #130344.
Shekhar Y C, Anand I S, Sarma R, Ferrari R, Wahi P L, Poole-Wilson P A. Effects of prolonged infusion of human alpha calcitonin gene-related peptide on hemodynamics, renal blood flow and hormone levels in congestive heart failure. Am J Cardiol 1991 Apr. 1; 67(8):732-736.
Stevenson R N, Roberts R H, Timmis A D. Calcitonin gene-related peptide: a haemodynamic study of a novel vasodilator in patients with severe chronic heart failure. Int J Cardiol December; 37(3):407-414 (1992).
Taylor C, Smith D, Hulce M, and Abel P. Pharmacological Characterization of Novel α-Calcitonin Gene-Related Peptide (CGRP) Receptor Peptide Antagonists That Are Selective for Human CGRP Receptors. JPET 319:749-757, (2006).
Zaidi, M et al. Structure activity relationship of human calcitonin-gene-related-peptide. Biochem. J., 269(3), 775-780 (1990).

All patents and publications referenced or mentioned herein are indicative of the levels of skill of those skilled in the art to which the invention pertains, and each such referenced patent or publication is hereby incorporated by reference to the same extent as if it had been incorporated by reference in its entirety individually or set forth herein in its entirety. Applicants reserve the right to physically incorporate into this specification any and all materials and information from any such cited patents or publications.

Concentrations, amounts, percentages, time periods, etc., of various components or use or effects of various components of this invention, including but not limited to sequence modified calcitonin gene related peptide (CGRP), are often presented in a range or baseline threshold format throughout this document. The description in range or baseline threshold format is merely for convenience and brevity and should not be construed as an inflexible limitation on the scope of the invention. Accordingly, the description of a range or baseline threshold should be considered to have specifically disclosed all the possible subranges as well as individual numerical values within that range or above that baseline threshold. For example, description of administration of CGRP in a range of about 2 to about 10 pmole/kg/min should be considered to have specifically disclosed subranges, such as about 3 to about 7 pmole/kg/min, about 4 to about 9 pmole/kg/min, about 3 to about 4 pmole/kg/min, etc., as well as individual numbers within that range, such as 2 pmole/kg/min, 4 pmole/kg/min, 8 pmole/kg/min, etc. This construction applies regardless of the breadth of the range or baseline threshold and in all contexts throughout this disclosure.

The foregoing description is considered as illustrative only of the principles of the invention. Further, since numerous modifications and changes will be readily apparent to those skilled in the art, it is not desired to limit the invention to the exact construction and process shown as described above. Accordingly, all suitable modifications and equivalents may be resorted to falling within the scope of the invention as defined by the statements of the invention that follow.

What is claimed is:

1. A biodegradable microparticle delivery system comprising biodegradable microparticles containing a sequence modified calcitonin gene related peptide having the sequence ACNTATCVTHRLAGLLSRSGGVVKSNFVPTNVGSKAF-NH₂ (SEQ ID NO:7) or a pharmaceutically acceptable salt thereof.

2. The microparticle delivery system of claim 1, wherein the biodegradable microparticles comprise poly(lactic-co-glycolic acid), poly(lactic acid), poly(caprolactone), polycarbonates, polyamides, polyanhydrides, polyamino acids, polyortho esters, polyacetals, polycyanoacrylates degradable polyurethanes, polyacrylates, ethylene-vinyl acetate copolymers, acyl substituted cellulose acetates or derivatives or copolymers thereof, or any combination thereof.

3. The microparticle delivery system of claim 1, wherein the microparticles are embedded in a gel matrix.

4. A sustained release delivery system comprising a biodegradable, water insoluble, thermoplastic polymer, a biocompatible solvent, and a sequence modified calcitonin gene related peptide having the sequence ACNTATCVTHRLAGLLSRSGGVVKSNFVPTNVGSKAF-NH₂ (SEQ ID NO:7) or a pharmaceutically acceptable salt thereof.

5. The delivery system of claim 4, wherein the biodegradable polymer is selected from the group consisting of polylactides, polyglycolides, polyanhydrides, polyorthoesters, polycaprolactones polyamides, polyurethanes, polyesteramides, polydioxanones, polyacetals, polyketals, polycarbonates, polyorthocarbonates, polyphosphazenes, polyhydroxybutyrates, polyhydroxyvalerates, polyalkylene oxalates, polyacrylates, polyalkylene succinates, poly(malic acid), poly(amino acids) and copolymers, terpolymers, cellulose diacetate, ethylene vinyl alcohol, and copolymers and combinations thereof.

6. A solid polymer matrix formed from the delivery system of claim 4, wherein the polymer matrix releases the sequence modified calcitonin gene related peptide or pharmaceutically acceptable salt thereof by diffusion, erosion, degradation, or a combination thereof as the polymer matrix biodegrades in the patient.

7. A pharmaceutical composition comprising the microparticle delivery system of claim 1 in combination with a pharmaceutical excipient, diluent, or carrier.

8. The pharmaceutical composition of claim 7, wherein the biodegradable microparticles comprise poly(lactic-co-glycolic acid), poly(lactic acid), poly(caprolactone), polycarbonates, polyamides, polyanhydrides, polyamino acids, polyortho esters, polyacetals, polycyanoacrylates degradable polyurethanes, polyacrylates, ethylene-vinyl acetate copolymers, acyl substituted cellulose acetates or derivatives or copolymers thereof, or any combination thereof.

9. The pharmaceutical composition of claim 8, wherein the biodegradable microparticles are embedded in a gel matrix.

10. The pharmaceutical composition of claim 8, wherein the sequence modified calcitonin gene related peptide or pharmaceutically acceptable salt thereof is encapsulated in a liposome.

11. Use of the sustained release delivery system of claim 4 for the manufacture of a medicament for treatment of a condition selected from the group consisting of heart failure, migraine, stroke, renal failure, advancement of heart failure into advanced stages, angina, hemorrhagic shock, pulmonary hypertension, ischemia due to a myocardial infarction, vasospasm during angioplasty, reclusion of blood vessels during and/or after angioplasty, stent insertion, implantation of a vascular grafts, male impotence and female sexual arousal disorder, wherein said medicament is administered to the patient at a frequency and for a duration of time effective to provide a beneficial effect to the patient.

12. The use of claim 11 wherein the medicament is delivered via a syringe, puncture needle, or catheter.

13. The use of claim 11, wherein the medicament is administered in conjunction with one or more drugs selected from the group consisting of anti-proliferative agents, anti-clotting agents, vasodilators, diuretics, beta-blockers, calcium ion channel blockers, blood thinners, cardiotonics, ACE inhibitors, anti-inflammatories, anti-platelet drugs, thrombolytic agents, antioxidants, and/or gene therapeutics.

14. The use of claim 11, wherein the medicament is administered by a method selected from parenteral, transdermal, intranasal, sublingual, transmucosal, intra-arterial, oral, intracoronary, intravenous, transmucosal, topical rectal, vaginal, and intradermal.

15. A kit comprising a first container comprising a controlled release formulation of sequence modified calcitonin gene related peptide or pharmaceutically acceptable salt thereof, the formulation comprising an amount of the peptide or pharmaceutically acceptable salt thereof effective to treat, reduce the risks, occurrence, or effects of heart failure, ischemia, myocardial infarction, hemorrhagic shock, angina, renal failure, stroke, vasospasms, male impotence, or female sexual arousal disorder, wherein the sequence modified calcitonin gene related peptide or pharmaceutically acceptable salt thereof has the amino acid sequence ACNTATCVTHRLAGLLSRSGGVVKSNFVPTNVGSKAF-NH₂ (SEQ ID NO:7).

16. The kit of claim 15, wherein the sequence modified CGRP or pharmaceutically acceptable salt thereof is present in a controlled release formulation.

17. The kit of claim 15, further comprising one or more drugs selected from the group consisting of anti-proliferative agents, anti-clotting agents, vasodilators, diuretics, beta-blockers, calcium ion channel blockers, blood thinners, cardiotonics, ACE inhibitors, anti-inflammatories, and antioxidants.

18. The kit of claim 15, further comprising a second container comprising one or more drugs selected from the group consisting of anti-proliferative agents, anti-clotting agents, vasodilators, diuretics, beta-blockers, calcium ion channel blockers, blood thinners, cardiotonics, ACE inhibitors, anti-inflammatories, antioxidants, and gene therapeutics.

19. The kit of claim 15, further comprising a syringe, puncture needle, or catheter.

20. A sustained release delivery system comprising a carrier and a sequence modified calcitonin gene related peptide having the sequence ACNTATCVTHRLAGLLSRSGGVVKSNFVPTNVGSKAF-NH₂ (SEQ ID NO:7), wherein the carrier provides sustained release of the sequence modified calcitonin gene related peptide.

21. The sustained release delivery system of claim 20, wherein the carrier comprises microparticles.

22. The sustained release delivery system of claim 20, wherein the carrier comprises a biodegradable thermoplastic polymer and a biocompatible solvent that forms a biodegradable implant when injected into a patient.

23. The sustained release delivery system of claim 20, wherein the sequence modified calcitonin gene related peptide (CGRP) is conjugated to polyethylene glycol.


SEQUENCE LISTING (60 sequences)

SEQ ID NO: 1 - protein, 37 aa, Homo sapiens
ACDTATCVTHRLAGLLSRSGGVVKNNFVPTNVGSKAF

SEQ ID NO: 2 - protein, 37 aa, Homo sapiens
ACNTATCVTHRLAGLLSRSGGMVKSNFVPTNVGSKAF

SEQ ID NO: 3 - protein, 37 aa, Artificial Sequence
  A synthetic peptide
ACNTATCVTHRLAGLLSRSGGVVKNNFVPTNVGSKAF

SEQ ID NO: 4 - protein, 37 aa, Artificial Sequence
  A synthetic peptide
ACDTATCVTHRLAGLLSRSGGMVKNNFVPTNVGSKAF

SEQ ID NO: 5 - protein, 37 aa, Artificial Sequence
  A synthetic peptide
ACDTATCVTHRLAGLLSRSGGVVKSNFVPTNVGSKAF

SEQ ID NO: 6 - protein, 37 aa, Artificial Sequence
  A synthetic peptide
ACNTATCVTHRLAGLLSRSGGMVKNNFVPTNVGSKAF

SEQ ID NO: 7 - protein, 37 aa, Artificial Sequence
  A synthetic peptide
ACNTATCVTHRLAGLLSRSGGVVKSNFVPTNVGSKAF

SEQ ID NO: 8 - protein, 37 aa, Artificial Sequence
  A synthetic peptide
ACDTATCVTHRLAGLLSRSGGMVKSNFVPTNVGSKAF

SEQ ID NO: 9 - protein, 37 aa, Artificial Sequence
  A synthetic peptide
ACNTATCVTHRLADLLSRSGGVVKNNFVPTNVGSKAF

SEQ ID NO: 10 - protein, 37 aa, Artificial Sequence
  A synthetic peptide
ACDTATCVTHRLADLLSRSGGMVKNNFVPTNVGSKAF

SEQ ID NO: 11 - protein, 37 aa, Artificial Sequence
  A synthetic peptide
ACDTATCVTHRLADLLSRSGGVVKSNFVPTNVGSKAF

SEQ ID NO: 12 - protein, 37 aa, Artificial Sequence
  A synthetic peptide
ACNTATCVTHRLADLLSRSGGMVKNNFVPTNVGSKAF

SEQ ID NO: 13 - protein, 37 aa, Artificial Sequence
  A synthetic peptide
ACNTATCVTHRLADLLSRSGGVVKSNFVPTNVGSKAF

SEQ ID NO: 14 - protein, 37 aa, Artificial Sequence
  A synthetic peptide
ACDTATCVTHRLADLLSRSGGMVKSNFVPTNVGSKAF

SEQ ID NO: 15 - protein, 37 aa, Artificial Sequence
  A synthetic peptide
FAKSGVNTPVFNNKVVGGSRSLLGALRHTVCTATNCA

SEQ ID NO: 16 - protein, 37 aa, Artificial Sequence
  A synthetic peptide
FAKSGVNTPVFNNKVMGGSRSLLGALRHTVCTATDCA

SEQ ID NO: 17 - protein, 37 aa, Artificial Sequence
  A synthetic peptide
FAKSGVNTPVFNSKVVGGSRSLLGALRHTVCTATDCA

SEQ ID NO: 18 - protein, 37 aa, Artificial Sequence
  A synthetic peptide
FAKSGVNTPVFNNKVMGGSRSLLGALRHTVCTATNCA

SEQ ID NO: 19 - protein, 37 aa, Artificial Sequence
  A synthetic peptide
FAKSGVNTPVFNSKVVGGSRSLLGALRHTVCTATNCA

SEQ ID NO: 20 - protein, 37 aa, Artificial Sequence
  A synthetic peptide
FAKSGVNTPVFNSKVMGGSRSLLGALRHTVCTATDCA

SEQ ID NO: 21 - protein, 37 aa, Artificial Sequence
  A synthetic peptide
FAKSGVNTPVFNNKVVGGSRSLLDALRHTVCTATNCA

SEQ ID NO: 22 - protein, 37 aa, Artificial Sequence
  A synthetic peptide
FAKSGVNTPVFNNKVMGGSRSLLDALRHTVCTATDCA

SEQ ID NO: 23 - protein, 37 aa, Artificial Sequence
  A synthetic peptide
FAKSGVNTPVFNSKVVGGSRSLLDALRHTVCTATDCA

SEQ ID NO: 24 - protein, 37 aa, Artificial Sequence
  A synthetic peptide
FAKSGVNTPVFNNKVMGGSRSLLDALRHTVCTATNCA

SEQ ID NO: 25 - protein, 37 aa, Artificial Sequence
  A synthetic peptide
FAKSGVNTPVFNSKVVGGSRSLLDALRHTVCTATNCA

SEQ ID NO: 26 - protein, 37 aa, Artificial Sequence
  A synthetic peptide
FAKSGVNTPVFNSKVMGGSRSLLDALRHTVCTATDCA

SEQ ID NO: 27 - protein, 37 aa, Artificial Sequence
  A synthetic peptide
FAKSGVNTPVFNNKVVGGSRSLLGALRHTVCTATDCA

SEQ ID NO: 28 - protein, 37 aa, Artificial Sequence
  A synthetic peptide
FAKSGVNTPVFNSKVMGGSRSLLDALRHTVCTATNCA

SEQ ID NO: 29 - protein, 37 aa, Artificial Sequence
  A synthetic peptide
FAKSGVNTPVFNSKVMGGSRSLLGALRHTVCTATNCA

SEQ ID NO: 30 - protein, 36 aa, Artificial Sequence
  A synthetic peptide
CNTATCVTHRLAGLLSRSGGVVKNNFVPTNVGSKAF

SEQ ID NO: 31 - protein, 36 aa, Artificial Sequence
  A synthetic peptide
CDTATCVTHRLAGLLSRSGGMVKNNFVPTNVGSKAF

SEQ ID NO: 32 - protein, 36 aa, Artificial Sequence
  A synthetic peptide
CDTATCVTHRLAGLLSRSGGVVKSNFVPTNVGSKAF

SEQ ID NO: 33 - protein, 36 aa, Artificial Sequence
  A synthetic peptide
CNTATCVTHRLAGLLSRSGGMVKNNFVPTNVGSKAF

SEQ ID NO: 34 - protein, 36 aa, Artificial Sequence
  A synthetic peptide
CNTATCVTHRLAGLLSRSGGVVKSNFVPTNVGSKAF

SEQ ID NO: 35 - protein, 36 aa, Artificial Sequence
  A synthetic peptide
CDTATCVTHRLAGLLSRSGGMVKSNFVPTNVGSKAF

SEQ ID NO: 36 - protein, 36 aa, Artificial Sequence
  A synthetic peptide
CNTATCVTHRLADLLSRSGGVVKNNFVPTNVGSKAF

SEQ ID NO: 37 - protein, 36 aa, Artificial Sequence
  A synthetic peptide
CDTATCVTHRLADLLSRSGGMVKNNFVPTNVGSKAF

SEQ ID NO: 38 - protein, 36 aa, Artificial Sequence
  A synthetic peptide
CDTATCVTHRLADLLSRSGGVVKSNFVPTNVGSKAF

SEQ ID NO: 39 - protein, 36 aa, Artificial Sequence
  A synthetic peptide
CNTATCVTHRLADLLSRSGGMVKNNFVPTNVGSKAF

SEQ ID NO: 40 - protein, 36 aa, Artificial Sequence
  A synthetic peptide
CNTATCVTHRLADLLSRSGGVVKSNFVPTNVGSKAF

SEQ ID NO: 41 - protein, 36 aa, Artificial Sequence
  A synthetic peptide
CDTATCVTHRLADLLSRSGGMVKSNFVPTNVGSKAF

SEQ ID NO: 42 - protein, 30 aa, Artificial Sequence
  A synthetic peptide
VTHRLAGLLSRSGGVVKNNFVPTNVGSKAF

SEQ ID NO: 43 - protein, 30 aa, Artificial Sequence
  A synthetic peptide
VTHRLAGLLSRSGGMVKSNFVPTNVGSKAF

SEQ ID NO: 44 - protein, 30 aa, Artificial Sequence
  A synthetic peptide
VTHRLADLLSRSGGVVKNNFVPTNVGSKAF

SEQ ID NO: 45 - protein, 30 aa, Artificial Sequence
  A synthetic peptide
VTHRLADLLSRSGGMVKSNFVPTNVGSKAF

SEQ ID NO: 46 - protein, 30 aa, Artificial Sequence
  A synthetic peptide
FAKSGVNTPVFNNKVVGGSRSLLGALRHTV

SEQ ID NO: 47 - protein, 30 aa, Artificial Sequence
  A synthetic peptide
FAKSGVNTPVFNSKVMGGSRSLLGALRHTV

SEQ ID NO: 48 - protein, 30 aa, Artificial Sequence
  A synthetic peptide
FAKSGVNTPVFNNKVVGGSRSLLDALRHTV

SEQ ID NO: 49 - protein, 30 aa, Artificial Sequence
  A synthetic peptide
FAKSGVNTPVFNSKVMGGSRSLLDALRHTV

SEQ ID NO: 50 - protein, 37 aa, Artificial Sequence
  A synthetic peptide
ACXTATCVTHRLAXLLSRSGGXVKXNFVPTNVGSKAF

SEQ ID NO: 51 - protein, 37 aa, Artificial Sequence; identical to SEQ ID NO: 50
  A synthetic peptide

SEQ ID NO: 52 - protein, 30 aa, Artificial Sequence
  A synthetic peptide
VTHRLAGLLSRSGGMVKNNFVPTNVGSKAF

SEQ ID NO: 53 - protein, 30 aa, Artificial Sequence
  A synthetic peptide
VTHRLAGLLSRSGGVVKSNFVPTNVGSKAF

SEQ ID NO: 54 - protein, 30 aa, Artificial Sequence
  A synthetic peptide
FAKSGVNTPVFNNKVMGGSRSLLGALRHTV

SEQ ID NO: 55 - protein, 30 aa, Artificial Sequence
  A synthetic peptide
FAKSGVNTPVFNSKVVGGSRSLLGALRHTV

SEQ ID NO: 56 - protein, 30 aa, Artificial Sequence
  A synthetic peptide
VTHRLADLLSRSGGMVKNNFVPTNVGSKAF

SEQ ID NO: 57 - protein, 30 aa, Artificial Sequence
  A synthetic peptide
VTHRLADLLSRSGGVVKSNFVPTNVGSKAF

SEQ ID NO: 58 - protein, 30 aa, Artificial Sequence
  A synthetic peptide
FAKSGVNTPVFNNKVMGGSRSLLDALRHTV

SEQ ID NO: 59 - protein, 30 aa, Artificial Sequence
  A synthetic peptide
FAKSGVNTPVFNSKVVGGSRSLLDALRHTV

SEQ ID NO: 60 - protein, 37 aa, Artificial Sequence
  A synthetic peptide
FAKSGVNTPVFNNKVVGGSRSLLDALRHTVCTATDCA